SEQUENCE LISTING (101 sequences)

SEQ ID NO: 1 - DNA, 2676 bp, Vaccinia virus
atgatgcctattaagtcaatagttactcttgatcaattagaggactctgaatatttatttcgtatagtttctaccgttcttccgcatctatgtctagattacaaagtatgtgaccaacttaaaacaaccttcgttcatccgttcgatatattgcttaataactcattaggatccgtaactaaacaagatgagcttcaggctgctatatccaaattgggcattaattatttaattgataccacgtcacgtgaattaaaactgtttaatgttacacttaacgctggaaatatagatattattaataccccaattaacattagttcggaaactaatcctatcattaatactcacagcttttacgatcttccacctttcactcaacaccttcttaatattagattgacggatacagaatacagagctagatttatcggtggttatattaaaccagatggctccgactcaatggatgttctagcagaaaagaaatatccagatcttaactttgataacacttatttgtttaacatcctctataaggatgttattaatgcaccaataaaagaattcaaggcaaaaattgttaacggtgtattaagcagacaagattttgataatcttataggtgttagacaatatataacaatacaagatcgaccccgctttgacgacgcttataacatcgcagatgctgctagacattatggagttaatcttaatacattgccattaccaaacgtcgatctcactactatgccaacatataaacatctcatcatgtttgaacagtacttcatttatacatatgacagagtggatatttattacaatggtaacaaaatgctcttcgatgatgagattataaactttactatttctatgcgatatcaatctcttattcctagactggtagatttctttccagatataccagtaaacaataacatcgtacttcatactcgcgatcctcaaaatgctgcagtgaatgtaaccgtggcgcttccaaacgtgcaatttgtggacataaatagaaacaacaaattctttattaatttctttaacctgttggcgaaggaacaaagatctacggctatcaaagttaccaaatccatgttttgggacggtatggattacgaggaatacaagtctaaaaaccttcaggacatgatgtttataaattctacctgttatgtattcggtctttataatcacaataatactacttattgctctatcctttctgatattatctccgcagagaaaacacctattagagtatgtttgttacccagagtagtcggaggtaagactgttactaatcttatttcagaaactttgaagagtatttcatctatgactatacgagagtttcccaggaaagataaatctatcatgcatataggactttctgagacgggattcatgagattcttccaactactcaggctcatggctgataaacctcatgaaacggctattaaagaggttgttatggcttatgtgggtataaagttgggtgacaaaggtagtccgtactatattagaaaggagtcataccaagactttatctatctgctattcgcatcaatgggctttaaggtgactactagaagatccattatgggaagcaataatatctctatcatcagtattagaccaagagtaactaaacaatacatcgtcgctacattgatgaaaactagttgtagtaaaaacgaggcagaaaaattgattacttcagcgtttgatcttctcaatttcatggtatcagttagtgactttagagattatcagagttacagacagtatagaaactattgtcctagatatttctatgcaggatctcccgaaggagaggaaaccattatctgtgactcggaaccgataagtatcttggatagaattgatactcgtggtatcttttctgcgtatactattaatgaaatgatggacactgatatcttttctccagagaataaggcatttaagaataatctgagtagatttatcgagagtggagatattacaggagaagatattttctgcgcaatgccatacaacatcttagataggattattacaaatgctggtacgtgtaccgtatccataggtgatatgttggataacattacaacccagtcagactgtaatatgactaacgaaatcacagatatgataaacgcctcattgaagaatacaatttctaaagataataatatgctagtcagccaagcgttgaactctgtagctaatcgttctaaacaaaagattggagacttgaggcaatcatcgtgtaagatggcattgttgtttaaaaatcttgctacatccatctacacaatagaacgtattttcaatgctaaagtaggcgatgatgttaaggcatcgatgttggagaagtataaagtattcacagatatttccatgtcattgtataaagacttgatagctatggagaatctcaaagcgatgctatacattattcgacgaagcggatgcagaatagacgatgcacaaattactactgacgatctagtcaagtcttactcattgatccgtcctaaaattctaagtatgataaactattataatgaaatgagtagaggatactttgaacacatgaaaaaaaatctaaatatgacagatggtgactctgtctcttttgatgatgaataa

SEQ ID NO: 2 - DNA, 957 bp, Vaccinia virus
atgacgaccgtaccagtgacggatatacaaaacgatttaattacagagttttcagaagataattatccatctaacaaaaattatgaaataactcttcgtcaaatgtctattctaactcacgttaacaacgtggtagatagagaacataatgccgccgtagtgtcatctccagaggaaatatcctcacaacttaatgaagatctatttccagatgatgattcaccggccactattatcgaacgagtacaacctcatactactattattgacgatactccacctcctacttttcgtagagagttattgatatcggaacaacgtcaacaacgagaaaaaagatttaatattacagtatcgaaaaatgctgaagcaataatggaatctagatctatgataacttctatgccaacacaaacaccatccttgggagtagtttatgataaagataaaagaattcagatgttggaggatgaagtggttaatcttagaaatcaacgatctaatacaaaatcatctgataatttagataattttaccaaaatactatttggtaagactccgtataaatcaacagaagttaataagcgtatagccatcgttaattatgcaaatttgaacgggtctcccttatcagtcgaggacttggatgtttgttcagaggatgaaatagatagaatctataaaacgattaaacaatatcacgaaagtagaaaacaaaaaattatcgtcactaacgtgattattattgtcataaacattatcgagcaagcattgctaaaactcggatttgaagaaatcaaaggactgagtaccgatatcacttcagaaattatcgatgtggagatcggagatgactgcgatgctgtagcatcaaaactaggaatcggtaacagtccggttcttaatattgtattgtttatactcaagatattcgttaaacgaattaaaattatttaa

SEQ ID NO: 3 - DNA, 213 bp, Vaccinia virus
atgattggtattcttttgttgatcggtatttgtgtagcagttaccgtcgccatcctatactcgatgtataataagatcaagaactcacaaaatccgaatccaagtccgaatttaaattcgcctcctccagaaccaaaaaataccaagtttgtaaataatctggaaaaggatcatattagttcattgtataatctagttaaatcttctgtataa

SEQ ID NO: 4 - DNA, 612 bp, Vaccinia virus
atgagttatttaagatattacaatatgcttgacgacttctctgcgggtgctggagtgcttgataaagatttatttacagaggaacagcagcaatcgtttatgcctaaagatggaggtatgatgcaaaacgattatggaggaatgaatgattatttgggaatcttcaaaaataatgatgttagaacgttactcggtttgattttgttcgtcttggctctatatagccctcctctaatctctatattgatgatatttatctcatcttttctattgcctcttactagcttagttattacctattgcttagtaactcaaatgtatcgtggaggtaatggcaacactgtgggaatgtctattgtgtgtattgtagctgctgtaattattatggcaatcaatgtatttacgaattcacagatatttaatattatttcttacattattttgtttattctgttctttgcatatgtgatgaacatcgaaagacaggactatagaagaagtataaatgtaaccattcctgaacagtatacctgcaacaaaccttatactgcgggaaataaggtagatgttgatataccaacatttaacagtttaaatactgacgattattaa

SEQ ID NO: 5 - DNA, 333 bp, Vaccinia virus
atggacggaactcttttccccggagatgacgatcttgcaattccagcaactgaatttttttctacaaaggctgctaaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgcccatatgagtaa

SEQ ID NO: 6 - DNA, 558 bp, Vaccinia virus
atgatgacaccagaaaacgacgaagagcagacatctgtgttctccgctactgtttacggagacaaaattcaaggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacacaatatgatcacaaagaaagctgtaatggtttatattaccagggttcttgttatatattacattcagactaccagttattctcggatgctaaagcaaattgcactgcggaatcatcaacactacccaataaatccgatgtcttgattacctggctcattgattatgttgaggatacatggggatctgatggtaatccaattacaaaaactacatccgattatcaagattctgatgtatcacaagaagttagaaagtatttttgtgttaaaacaatgaactaa

SEQ ID NO: 7 - DNA, 1935 bp, Vaccinia virus
atggaagccgtggtcaatagcgatgtttttttaacatctaacgcaggactaaaatctagttatactaatcaaactctttctttggtagatgaagatcatattcacacttctgataaatctttgtcttgtagtgtatgcaattcattgtcccaaattgtagacgatgactttatatccgcaggggctagaaatcaacgtaccaaacctaaacgtgcaggaaataatcaatctcaacagcctatcaaaaaggattgtatggtttccatcgacgaagtagcatctacacatgattggagtacgagattgagaaatgatgggaatgcaattgctaaatatctaactactaacaagtatgacacatctaactttactattcaggatatgcttaacattatgaataaactaaatattgtcagaacaaatagaaacgagctatttcaactccttacccatgtaaagagcacattgaacaatgctagtgtttctgtgaaatgtactcatcctttagtacttattcattctcgagctagtcctagaatcggtgaccaactcaaagagttagataaaatatactctccatctaatcatcatattcttctgtcgactacacgattccaatccatgcattttaccgatatgtctagttcacaagatttgtcttttatttatagaaaaccagaaactaattactatattcatcctattctgatggcactattcggtattaaacttcctgcgctcgagaacgcgtatgtacatggagacacctatagcctaatccagcaactttatgaatttagaaaagtaaagtcttataattatatgttgttggttaatcgtcttacggaggataatccgatagtgattacaggtgtatcagatctaatttccacagagattcagagagcaaacatgcataccatgattagaaaagcaattatgaacattagaatgggaattttttattgtaacgatgatgatgcggtagatccccatctaatgaagattattcatactggatgctctcaagttatgacagatgaggaacagatattggcttctattttgtctatagttggatttagacctacgttggtttctgtggctagacctataaacggcatcagttacgatatgaaacttcaggcggcaccatacatagttgttaatcctatgaagatgatcacaacatccgacagtccgatttctatcaattccaaggatatttattctatggcattcgatggcaatagtggaagagtggtgttcgctcctcctaacataggatatggaagatgttctggagttacacacattgatccattgggaactaatgtgatgggtagtgctgttcattcccctgttatcgttaatggagcaatgatgttttatgtagaacgacgtcagaataagaatatgtttggtggagaatgttacaccggctttagatctctaatagatgatactccgattgacgtatcaccagaaatcatgctaaacggtatcatgtataggttaaagtccgcagtttgttacaaactcggagaccaattctttgattgtggatcgtctgatatcttcttgaagggacattatacgattctatttacagaaaatggaccctggatgtacgatcctctttctgttttcaatccgggagctagaaatgctagattgatgcgagctctcaaaaaccagtacaagaaattatcaatggattcagacgatggtttttatgaatggttgaatggcgacggttcagtatttgctgcctcaaaacagcaaatgttgatgaatcacgttgctaactttgacgacgatcttctaactatggaagaagccatgtcgatgatttcgagacattgttgtatcttaatttatgcacaggattatgatcaatatattagcgctagacatattacagaactattttag

SEQ ID NO: 8 - DNA, 660 bp, Vaccinia virus
atgtactcgttagtatttgttattttgatgtgtataccatttagttttcaaacagtgtatgatgataaatcggtatgcgattctgacaataaagaatatatgggaatagaagtttatgtagaagcaacgctagacgaacccctcagacaaacaacgtgtgaatccaaaatccataaatatggtgcatctgtatcaaacggaggattaaatatttctgttgatctattaaactgttttcttaattttcatacagttggtgtatacactaatcgcgataccgtatacgcgaagtttgctagtttggatccatggactacggaacctataaattctatgacccatgacgatctagtaaaattaacagaagaatgtatagtggacatttatttaaaatgtgaagtggataaaacaaaggatttcatgaaaactaacggtaatagattaaaaccaagagactttaaaactgttcctccttctaatgtaggaagcatgatagaactacagtctgactattgcgtaaacgatgtgactacatacgtcaaaatatacgatgagtgtggaaacattaaacagcattccattccaacactaagagattattttaccaccaagaatggtcaaccacgtaaaatattaaagaaaaaatttgataattgttaa

SEQ ID NO: 9 - DNA, 759 bp, Vaccinia virus
atgggtaacaaaaatattaaaccatctaaggaaaatagactgtccatcttgtccaaggataagatggattcatttaagagaggatcttgggcaacgtcatcctttagagaaaagtcgcgtgcaaccatccaaagattttcatctcttagacgagaacatattaaagtagaccatcctgacaagttcctggagttaaagagagggatatataaaataattcagaaatcgtcgtctatagatgtggacaaacggactaagctcatgtccaacataaaaacgatgatgataaatccattcatgatcgagggtttaatgacatctttagaaaacttggatcccgataacaagatgagctactcatcggtgatgatattgggagaattcgacatcatcaatataagcgacaatgaggcggcattcgagttcataaacagtctgttgaaatctcttctcttgttaaatactagacaactaaaactcttggaatactccattagtaatgacttgttgtatgcccacataaatgcgttggagtatatcataaaaaatacatttaatgttccagaacggcaactgattctgagaggtcaatacctaactccaattttcagtgatttgttaaagtatgcgggtctaaccataaagtcaaacatactcatgtggaataaacagtttatcaaaccagtatctgacctctatacatctatgagactccttcattgtgttacagaatcatataaggtgattggaatgggataa

SEQ ID NO: 10 - DNA, 846 bp, Vaccinia virus
atggacttctttaacaagttctcacaggggctggcagaatcctctacaccaaagtcgtcaatctattattctgaagaaaaggatccggatacgaaaaaggatgaagcgattgaaataggactaaagtctcaagagtcgtattatcaaagacagttgcgagaacaactagctagagataatatgacggtcgccagcagacagcctatccaaccgctacaaccaactattcatataactccacagccggttccaacagctacaccggctcctattcttctacctagtagtactgttcctacaccaaaaccacgacaacaaactaatacatcatctgatatgtctaatctttttgattggctgtctgaagatactgatgcgccggcgagttcactccttccagcgttgacgccgagcaatgctgttcaggatattatctctaaatttaataaagatcaaaagacgacgacaccgccatctacccaaccttctcagacgttaccaacaactacatgtacacaacaatcggatggaaatatttcttgtactactccaacggttacacctcctcaacctcctattgtggccactgtatgtactcctacacctactggtggtacagtatgtacaacagcacaacaaaatccaaatccaggagcagcatctcaacaaaatctagacgatatggcccttaaggatctcatgtcgaatgttgaaagagatatgcaccaacttcaggccgaaacaaacgatctggtgacgaacgtatatgatgcaagggagtatacgcgtagggcaatagatcaaattctacaactagtcaaaggttttgaacgattccaaaagtaa

SEQ ID NO: 11 - DNA, 945 bp, Vaccinia virus
atgacacgattaccaatacttttgttactaatatcattagtatacgctacaccttttcctcagacatctaaaaaaataggtgatgatgcaactctatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcaactacaaatgacactgataaagtagattatgaagaatactccacagagttgattgtaaatacagatagtgaatcgactatagacataatactatctggatctacacattcaccggaaactagttctaagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagatcatacagttacagacactgtctcatacactacagtaagtacatcatctggaattgtcactactaaatcaaccaccgatgatgcggatctttatgatacgtacaatgataatgatacagtaccaccaactactgtaggcggtagtacaacctctattagcaattataaaaccaaggactttgtagaaatatttggtattaccgcattaattatattgtcggccgtggcaattttctgtattacatattatatatataataaacgttcacgtaaatacaaaacagagaacaaagtctag

SEQ ID NO: 12 - DNA, 660 bp, Vaccinia virus
atggcgatgttttacgcacacgctctcggtgggtacgacgagaatcttcatgcctttcctggaatatcatcgactgttgccaatgatgtcaggaaatattctgttgtgtcagtttataataacaagtatgacattgtaaaagacaaatatatgtggtgttacagtcaggtgaacaagagatatattggagcactgctgcctatgtttgagtgcaatgaatatctacaaattggagatccgatccatgatcaagaaggaaatcaaatctctatcatcacatatcgccacaaaaactactatgctctaagcggaatcgggtacgagagtctagacttgtgtttggaaggagtagggattcatcatcacgtacttgaaacaggaaacgctgtatatggaaaagttcaacatgattattctactatcaaagagaaggccaaagaaatgaatgcacttagtccaggacctatcattgattaccacgtctggataggagattgtatctgtcaagttactgctgtggacgtacatggaaaggaaattatgagaatgagattcaaaaagggtgcggtgcttccgatcccaaatctggtaaaagttaaacttggggagaatgatacagaaaatctttcttctactatatcggcggcaccatcgaggtaa

SEQ ID NO: 13 - DNA, 954 bp, Vaccinia virus
atgaaaacgatttccgttgttacgttgttatgcgtactacctgctgttgtttattcaacatgtactgtacccactatgaataacgctaaattaacgtctaccgaaacatcgtttaatgataaacagaaagttacgtttacatgtgatcagggatatcattcttcggatccaaatgctgtctgcgaaacagataaatggaaatacgaaaatccatgcaaaaaaatgtgcacagtttctgattacatctctgaattatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatatgccgtctctatctaatggattaatttccggatctacattttctatcggtggcgttatacatcttagttgtaaaagtggttttacactaacggggtctccatcatccacatgtatcgacggtaaatggaatcccgtactcccaatatgtgtacgaactaacgaagaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaa

SEQ ID NO: 14 - DNA, 747 bp, Vaccinia virus
atgaacttttacagatctagtataattagtcagattattaagtataatagacgactagctaagtctattatttgcgaggatgactctcaaattattacactcacggcattcgttaaccaatgcctatggtgtcataaacgagtatccgtgtccgctattttattaactactgataacaaaatattagtatgtaacagacgagatagttttctctattctgaaataattagaactagaaacatgtttagaaagaaacgattatttctgaattattccaattatttgaacaaacaggaaagaagtatactatcgtcatttttttctctagatccagctactgctgataatgatagaatagacgctatttatccgggtggcatacccaaaaggggtgagaatgttccagagtgtttatccagggaaattaaagaagaagttaatatagacaattcttttgtattcatagacactcggttttttattcatggcatcatagaagataccattattaataaattttttgaggtaatcttctttgtcggaagaatatctctaacgagtgatcaaatcattgatacatttaaaagtaatcatgaaatcaaggatctaatatttttagatccgaattcaggtaatggactccaatacgaaattgcaaaatatgctctagatactgcaaaactcaaatgttatggccatagaggatgttattacgaatcattaaaaaaattaactgaggatgattga

SEQ ID NO: 15 - DNA, 1896 bp, Vaccinia virus
atgagtaaatcacacgcggcctatatcgattatgcattgcgcagaactactaatatgcctgttgaaatgatggggtcggacgtagtacgcctcaaggattatcaacattttgtagcaagagttttcttaggattagacagtatgcattctcttttattgttccatgaaacgggtgtcggtaaaacaatgactactgtatatattctcaaacatcttaaggatatttatacgaattgggctattatcttattggtgaaaaaggctttgatagaagatccttggatgaacactatactcagatacgctccagagataacgaaggattgtatttttattaattacgatgatcaaaattttagaaataaattttttactaatatcaaaactattaattccaagagtagaatatgcgtcattattgatgaatgtcataacttcatttctaaatcattaatcaaagaagatggtaagatccgtcctactcgttcagtatataattttttatctaagaccatcgcattaaaaaaccataagatgatttgtttatcggctacacctatcgtcaatagtgtgcaagaattcaccatgttggttaacttactacgaccaggatccttacaacaccaatcgctatttgagaataaacgtctagttgatgaaaaagaattagtctccaaactaggaggcctatgttcgtacatagttaataacgagttttctatttttgatgacgtagaagggtctgcatcattcgctaagaaaacagtattaatgcgatacgttaatatgtcgaaaaagcaagaagaaatttatcaaaaggctaaactcgctgaaataaaaacaggtatatcatcatttagaattctgagacgtatggctactacgtttacgttcgatagctttcctgaaagacaaaatcgtgatccgggcgaatacgcgcaagagatagcaacactatataatgattttaaaaattcattaagagatagagaattttctaaatctgcattagatacctttaaaaggggagaactattgggaggggatgctagtgcggctgatatctctctatttactgaattaaaagagaaaagcgtcaaatttatagatgtatgtttgggaatattagcatcccatggtaaatgtctagtctttgaaccatttgttaatcagtcaggaatagaaatcttattactatatttcaaagtctttggtatctctaatatagagttctcatctagaacaaaagatactagaatcaaggcggtggctgagtttaaccaagaatcaaacactaacggagaatgcattaaaacatgcgtattctcttctagtggaggcgagggtattagcttcttctcaatcaatgatatcttcattttagatatgacatggaacgaggcgtctcttcgtcagatagtaggaagagccattcgtctcaatagtcacgttcttactcctccagaacgtagatatgtaaacgtgcactttataatggctagattatctaatggtatgcctactgtagacgaagacctatttgaaatcattcaaagcaaatcaaaagaatttgtccaattgtttagagtgtttaaacatacatcattagaatggattcatgctaatgaaaaagacttctcaccgatcgacaatgagtccggttggaaaaccttggtttcaagagccatcgatctatcgtctaacaaaaatattaccaataaactaattgagggtactaatatttggtattccaattctaatagattaatgtcaataaatagaggatttaaaggcgtagatggtcgagtatacgatgtagacggtaactatctacatgatatgccggacaatcccgttataaaaatacacgatggtaaattaatttatattttctaa

SEQ ID NO: 16 - DNA, 1656 bp, Vaccinia virus
atgaataatactatcattaattctttgatcggtggggatgactctattaaacggtctaatgtcttcgcagtcgatagtcaaattccaactttatatatgccgcaatatatttctctatccggagttatgacaaacgatggtccagacaatcaggctatcgctagcttcgaaattagggatcagtatattactgcgcttaatcatttggttctgagtttggaacttccagaagttaaaggtatgggaagattcggttacgtaccatatgttggatataaatgtattaatcacgtatctatctcttcgtgtaacggtgttatttgggaaattgagggcgaagaattatataataattgtatcaataatacaattgctttgaaacactctggatattctagtgaacttaatgatatttctattggcctaactcctaatgacactattaaagaaccatctacagtatacgtttatattaaaactccgtttgatgtggaagatacattcagcagtcttaaactatccgattcaaaaattaccgtaacggtaaccttcaatccagtatccgatatcgttattcgtgactcttcgttcgactttgaaacgttcaacaaagaatttgtttatgttcctgaattgagctttattggatatatggttaagaatgtacaaattaaaccatcatttatagagaaacctaggagagtaataggtcaaataaaccaaccaacggcgactgtaactgaagttcatgcggcaacatcgctctctgtttatactaaaccttattatggaaatacggataataaatttatttcgtatccagggtactcacaagatgaaaaagattatatagatgcatatgtgagtagattgttggatgatctagttattgttagcgatggtccaccgactggttatccggagtctgccgagatcgtagaggttccagaagatggtatcgtttctattcaagatgctgatgtgtatgtaaaaattgataatgttcctgataatatgagtgtttatcttcatactaatctgctaatgtttggaacacgaaaaaattcttttatatataacatttctaaaaagttttccgccattactggaacatatagtgatgccactaagagaacaatctttgctcacatatcacatagtatcaacatcatcgatacatctattcctgtaagtctttggactagtcaacgtaacgtctataacggagataatagatcagccgaatcaaaggccaaggatttgttcattaacgatcccttcatcaagggaatagattttaagaataagaccgatattatttctagactagaagttagatttggaaatgatgttctatattcagagaacggacccatctcgagaatttataatgaactactgacaaaaagcaataatggaacaagaaccctaacttttaactttacaccaaagatattctttaggccgacaactattacggctaatgtatctagggggaaagataaactatctgttcgagtagtttattccaccatggatgtcaaccatccaatctattatgtacaaaaacaattggtagttgtatgtaatgacctgtataaggtatcttacgatcaaggggtaagtattaccaagattatgggagataataactaa

SEQ ID NO: 17 - DNA, 915 bp, Vaccinia virus
atgccgcaacaactatctcctattaatatagaaactaaaaaagcaatttctaacgcgcgattgaagccgttagacatacattataatgagtcgaaaccaaccactatccagaacactggaaaactagtaaggattaattttaaaggaggatatataagtggagggtttctccccaatgaatatgtgttatcatcactacatatatattggggaaaggaagacgattatggatccaatcacttgatagatgtgtacaaatactctggagagattaatcttgttcattggaataagaaaaaatatagttcttatgaagaggcaaaaaaacacgatgatggacttatcattatttctatattcttacaagtattggatcataaaaatgtatattttcaaaagatagttaatcaattggattccattagatccgccaatacgtctgcaccgtttgattcagtattttatctagacaatttgctgcctagtaagttggattattttacatatctaggaacaactatcaaccactctgcagacgctgtatggataatttttccaacgccaataaacattcattctgatcaactatctaaattcagaacactattgtcgtcgtctaatcatgatggaaaaccgcattatataacagagaactatagaaatccgtataaattgaacgacgacacgcaagtatattattctggggagattatacgagcagcaactacctctccagcgcgcgagaactattttatgagatggttgtccgatttgagagagacatgtttttcatattatcaaaaatatatcgaagagaataaaacattcgcaattattgccatagtattcgtgtttatacttaccgctattctcttttttatgagtcgacgatattcgcgagaaaaacaaaactag

SEQ ID NO: 18 - DNA, 1440 bp, Vaccinia virus
atgaataggaatcctgatcagaatactcttcctaatattacattaaagattatagaaacctatttaggcagagtacctagtgtgaacgaatatcatatgttaaaattacaagctagaaatattcaaaaaataactgtttttaacaaagacatatttgtatctttagtaaaaaagaataaaaaaagatttttttccgatgttaatacatctgcatcagaaataaaagatcgtatacttagctacttttctaaacagactcaaacatataatataggtaaattatttacgattatagaactacaatctgtattagtgaccacatacacggacatattaggagttcttactattaaagctccaaatgtaatttcatctaaaatttcttataatgtaacatcaatggaagaattggcaagagatatgctaaattctatgaacgtcgcagtaatagacaaggcaaaagtaatgggacgtcataatgtatcttccctagtcaaaaatgttaataagttgatggaagaatatcttagacgccataataaaagttgtatatgttacggatcatattctctatatctaattaatccaaatatacggtacggcgatatagatattcttcagactaattctagaacttttcttatagatttggcctttctaataaaatttatcacgggaaataatattatattaagtaaaatcccatatcttagaaactatatggtgataaaagatgaaaacgataatcatatcattgatagttttaatattcgccaggataccatgaacgtagttcctaaaatctttatagataatatctatatagtggatccgacgtttcaactattgaacatgataaaaatgttttctcaaatagatagattggaagatctatccaaagatcctgaaaagtttaatgcgcgtatggcaaccatgctagaatacgttagatatacacatggtatagtctttgatggtaagcgtaataatatgccgatgaaatgtatcatcgatgaaaataatcgcatagttactgtcactactaaagactattttagctttaaaaaatgtctagtgtatctagatgaaaatgtgttatcgagtgatatattagatcttaacgccgacacatcgtgtgatttcgagagtgttacaaattctgtatatctaattcatgataatatcatgtatacatatttctcaaatactattctccttagtgataaggggaaggtacatgaaataagtgccagaggtttatgtgcacatatattgttgtatcagatgctgacatctggagaatacaaacaatgtttatcggatctcttaaattcgatgatgaatagagataaaatacctatctattcacatactgaaagagataaaaaacctggacgacacggatttattaatatcgaaaaggatataattgtattttag

SEQ ID NO: 19 - DNA, 573 bp, Vaccinia virus
atgtctaaaatctatatcgacgagcgttctaacgcagagattgtgtgtgaggctattaaaaccattggaatcgaaggagctactgctgcacaactaactagacaacttaatatggagaagcgagaagttaataaagctctgtacgatcttcaacgtagtgctatggtgtacagctccgacgatattcctcctcgttggtttatgacaacggaggcggataagccggatgctgatgctatggctgacgtcataatagatgatgtatcccgcgaaaaatcaatgagagaggatcataagtcttttgatgatgttattccggctaaaaaaattattgattggaaaggtgctaaccctgtcaccgttattaatgagtactgccaaattactaggagagattggtcttttcgtattgaatcagtggggcctagtaactctcctacattttatgcctgtgtagacatcgacggaagagtattcgataaggcagatggaaaatctaaacgagatgctaaaaataatgcagctaaattggcagtagataaacttcttggttacgtcatcattagattctga

SEQ ID NO: 20 - DNA, 1119 bp, Vaccinia virus
atgtggccatttgcatcggtacctgcgggagcaaaatgtaggctggtagaaacactaccagaaaatatggattttagatccgatcatttaacaacatttgaatgttttaacgaaattatcactctagctaagaaatatatatacatagcatctttttgttgtaatcctctgagtacgactaggggagcgcttatttttgataaactaaaagaggcatctgaaaaagggattaaaataatagttttgctagatgaacgagggaaaagaaatctgggagagctacaaagtcactgcccggatataaattttataaccgttaatatagataaaaaaaataatgtgggactactactcggttgtttttgggtgtcagatgatgaaagatgttatgtaggaaacgcgtcatttactggaggatctatacatacgattaaaacgttaggtgtatattctgattatcccccgctggccacagatcttcgtagaagatttgatacttttaaagcctttaatagcgcaaaaaattcatggttgaatttatgctctgcggcttgttgtttgccagttagcactgcgtatcatattaagaatcctataggtggagtgttctttactgattctccggaacacctattgggatattctagagatctagataccgatgtagttattgataaactcaagtcggctaagactagtatagatattgaacatttggccatagttcccactacacgtgtcgacggtaatagctactattggcccgacatttacaactccattatagaagcagccattaatagaggagttaagatcagacttctagttggtaattgggataagaacgacgtatattctatggcaaccgccagaagtctagacgcgttgtgtgttcaaaatgatctatctgtgaaggttttcactattcagaataatacaaaattgttgatagtcgacgacgaatatgttcatatcacttcggcaaatttcgacggaacccattaccaaaatcacggattcgtcagttttaatagtatagataaacagcttgtaagcgaggctaaaaaaatatttgagagagattgggtatctagccacagtaaatcgttaaaaatttaa

SEQ ID NO: 21 - DNA, 444 bp, Vaccinia virus
atgttcaacatgaatattaactcaccagttagatttgttaaggaaactaacagagctaaatctcctactaggcaatcaccttacgccgccggatatgatttatatagcgcttacgattatactatccctccaggagaacgacagttaattaagacagatattagtatgtccatgcctaagttctgctatggtagaatagctcctaggtctggtctgtccctaaaaggcattgatataggaggcggtgtaatagacgaagattataggggaaacataggagtcattcttattaataatggaaaatgtacgtttaatgtaaatactggagatagaatagctcagctaatctatcaacgtatatattatccagaactggaagaagtacaatctctagatagtacaaatagaggagatcaagggtttggatcaacaggacttagataa

SEQ ID NO: 22 - DNA, 639 bp, Vaccinia virus
atggcggaaactaaagagtttaaaactttgtataatctttttatagatagttatttacaaaaattagctcaacattctatccctactaatgtcacttgtgctattcatataggagaggttataggacagtttaaaaattgcgcgctccgaataactaacaaatgcatgagtaattctcgacttagtttcacactcatggttgaatcatttattgaagtgatttcattgcttccggaaaaggatagaagagctatcgctgaagaaataggaatagatctagacgatgtacctagtgcggtatccaagctagaaaagaactgtaatgcgtatgcggaggttaataatattatagatatacagaaattagatatcggagaatgttcggctccgcccggtcaacatatgcttttacagatagttaatacaggatccgcggaagcaaattgtggtttacagacaattgttaagtccttaaataaaatatacgttccacctattatcgaaaaccgattgccgtattacgatccgtggtttctagtgggtgtagcaattattctagttatttttactgtagctatttgttctattagacgaaatctggctcttaaatacagatacggaacgtttttatacgtttaa

SEQ ID NO: 23 - DNA, 1305 bp, Vaccinia virus
atgggtatcaaaaacttaaaatcgttactgctggaaaataaatcactgacgatattagatgataatttatacaaagtatacaatggaatatttgtggatacaatgagtatttatatagccgtcgccaattgtgtcagaaacttagaagagttaactacggtattcataaaatacgtaaacggatgggtaaaaaagggagggcatgtaaccctttttatcgatagaggaagtataaaaattaaacaagacgttagagacaagagacgtaaatattctaaattaaccaaggacagaaaaatgctagaattagaaaagtgtacatccgaaatacaaaatgttaccggatttatggaagaagaaataaaggcagaaatgcaattaaaaatcgataaactcacatttcaaatatatttatctgattctgataacataaaaatatcattgaatgagatactaacacatttcaacaataatgagaatgttacattattttattgtgatgaacgagacgcagaattcgttatgtgtctcgaggctaaaacacatttctctaccacaggagaatggccgttgataataagtaccgatcaggatactatgctatttgcatctgctgataatcatcctaagatgataaaaaacttaactcaactgtttaaatatgttccatctgcagaggataactatttagcaaaattaacggcgttagtgaatggatgtgatttctttcctggactctatggggcatctataacacccaacaacttaaacaaaatacaattgtttagtgattttacaatcgataatatagtcactagtttggcaattaaaaattattatagaaagactaactctaccgtagacgtgcgtaatattgttacgtttataaacgattacgctaatttagacgatgtctactcgtatattcctccttgtcaatgcactgttcaagaatttatattttccgcattagatgaaaaatggaatgaatttaaatcatcttatttagaaagcgtgccgttaccctgccaattaatgtatgcgttagaaccacgcaaggagattgatgtttcagaagttaaaactttatcatcttatatagatttcgaaaatactaaatcagatatcgatgttataaaatctatatcctcgatcttcggatattctaacgaaaactgtaacacgatagtattcggcatctataaggataatttactactgagtataaatagttcattttactttaacgatagtctgttaataaccaatactaaaagtgataatataataaatataggttactag

SEQ ID NO: 24 - DNA, 1116 bp, Vaccinia virus
atggctgcagaacagcgtcgttctacaatttttgacatagtttcaaaatgtatagtgcaatctgtattaagagatatatctattaattctgaatacatagagtccaaagctaaacaattgtgctattgtccggcatcgaaaaaggaatcggtgattaatggtatctacaattgttgcgagtcaaatatagaaataatggacaaagagcagctattaaaaatattggacaatcttcgatgtcattcggctcatgtatgtaacgccacagatttctggagactatataattcgttaaaacggtttactcatactaccgcattctttaatacatgcaagcccactattctagccacgctaaacactttgataaccctgattttatctaacaagttattgtatgcggcagaaatggtagagtatctagagaaccaactagattcatcaaataaatcaatgtctcaagaactagcagaattattggaaatgaaatatgctctcattaatctggtacaatataggattttgccaatgatcatcggtgagcctattatagtagctggattttctggtaaagaaccaatttctgattattctgcagaagtggaaaggctaatggaactaccagttaaaactgatatagtgaataccacatatgacttcttagccagaaaaggtattgatactagcaacaatatagcagaatatatagccggcttgaaaatagaagagattgaaaaggtagaaaaatatttaccagaagttatatctacaattgccaatagtaatataataaaaaataaaaaatctatctttccggccaatatcaacgataaacagatcatggaatgctctagaatgttagacacgagtgagaaatactctaaaggatataaaactgatggagctgtgactagtccattgacgggaaataatacaattacaacatttataccaatttctgcgtccgatatgcaaaagtttaccattttagaatatctttacattatgagagtgatggcaaacaacgttaagaaaaagaacgagggaaaaaacaacggaggagtagttatgcatattaactcaccctttaaggtaatcaatttgccaaaatgttaa

SEQ ID NO: 25 - DNA, 975 bp, Vaccinia virus
atggcggcggcgaaaactcctgttattgttgtgccagttattgatagacttccatcagaaacatttcctaatgttcatgagcatattaatgatcagaagttcgatgatgtaaaggacaacgaagttatgccagaaaaaagaaatgttgtggtagtcaaggatgatccagatcattacaaggattatgcgtttatacagtggactggaggaaacattagaaatgatgacaagtatactcacttcttttcagggttttgtaacactatgtgtacagaggaaacgaaaagaaatatcgctagacatttagccctatgggattctaatttttttaccgagttagaaaataaaaaggtagaatatgtagttattgtagaaaacgataacgttattgaggatattacgtttcttcgtcccgtcttgaaggcaatgcatgacaaaaaaatagatatcctacagatgagagaaattattacaggcaataaagttaaaaccgagcttgtaatggacaaaaatcatgccatattcacatatacaggagggtatgatgttagcttatcagcctatattattagagttactacggcgctgaacatcgtagatgaaattataaagtctggaggtctatcatcgggattttattttgaaatagccagaattgaaaacgaaatgaagatcaataggcagatactggataatgccgccaaatatgtagaacacgatccccgacttgttgcagaacaccgtttcgaaaacatgaaaccgaatttttggtctagaataggaacggcagctactaaacgttatccaggagttatgtacgcgtttactactccactgatttcattttttggattgtttgatattaatgttataggtttgattgtaattttgtttattatgtttatgctcatctttaacgttaaatctaaactgttatggttccttacaggaacattcgttaccgcatttatctaa

SEQ ID NO: 26 - DNA, 612 bp, Vaccinia virus
atggcgtggtcaattacaaataaagcggatactagtagcttcacaaagatggctgaaatcagagctcatctaaaaaatagcgctgaaaataaagataaaaacgaggatattttcccggaagatgtaataattccatctactaagcccaaaaccaaacgagccactactcctcgtaaaccagcggctactaaaagatcaaccaaaaaggaggaagtggaagaagaagtagttatagaggaatatcatcaaacaactgaaaaaaattctccatctcctggagtcagcgacattgtagaaagcgtggccgctgtagagctcgatgatagcgacggggatgatgaacctatggtacaagttgaagctggtaaagtaaatcatagtgctagaagcgatctctctgacctaaaggtggctaccgacaatatcgttaaagatcttaagaaaattattactagaatctctgcagtatcgacggttctagaggatgttcaagcagctggtatctctagacaatttacttctatgactaaagctattacaacactatctgatctagtcaccgagggaaaatctaaagttgttcgtaaaaaagttaaaacttgtaagaagtaa

SEQ ID NO: 27 - DNA, 945 bp, Vaccinia virus
atgcgtgcacttttttataaagatggtaaactctttaccgataataattttttaaatcctgtatcagacgataatccagcgtatgaggttttgcaacatgttaaaattcctactcatttaacagatgtagtagtatatgaacaaacgtgggaggaggcgttaactagattaatttttgtgggaagtgattcaaaaggacgtagacaatacttttacggaaaaatgcatgtacagaatcgcaacgctaaaagagatcgtatttttgttagagtatataacgttatgaaacgaattaattgttttataaacaaaaatataaagaaatcgtccacagattccaattatcagttggcggtttttatgttaatggaaactatgttttttattagatttggtaaaatgaaatatcttaaggagaatgaaacagtagggttattaacactaaaaaataaacacatagaaataagtcccgatgaaatagttatcaagtttgtaggaaaggacaaagtttcacatgaatttgttgttcataagtctaatagactatataagccgctattgaaactgacggatgattctagtcccgaagaatttctgttcaacaaactaagtgaacgaaaggtatatgaatgtatcaaacagtttggtattagaatcaaggatctccgaacgtatggagtcaattatacgtttttatataatttttggacaaatgtaaagtccatatctcctcttccatcaccaaaaaagttaatagcgttaactatcaaacaaactgctgaagtggtaggtcatactccatcaatttcaaaaagagcttatatggcaacgactattttagaaatggtaaaggataaaaattttttagatgtagtatctaaaactacgttcgatgaattcctatctatagtcgtagatcacgttaaatcatctacggatggatga

SEQ ID NO: 28 - DNA, 441 bp, Vaccinia virus
atggaaatggataagcgtatgaaatctctcgcaatgaccgctttctttggggagctaagcacattagatattatggcattgataatgtctatatttaaacgccatccaaacaataccattttttcagtggataaggatggtcagtttatgattgatttcgaatacgataattataaggcttctcaatatttggatctgaccctcactccgatatctggagatgaatgcaagactcacgcatcgagtatagccgaacaattggcgtgtgtggatattattaaagaggatattagcgaatatatcaaaactactccccgtcttaaacgatttataaaaaaataccgcaatagatcagatactcgcatcagtcgagatacagaaaagcttaaaatagctctagctaaaggcatagattacgaatatataaaagacgcttgttaa

SEQ ID NO: 29 - DNA, 939 bp, Vaccinia virus
atggcggaatttgaagatcaactcgttttcaatagtatcagtgcccgtgcattgaaagcttatttcactgctaaaatcaatgaaatggtagatgagttggtcacaagaaaatgtccacaaaagaaaaaatcacaagctaagaaacctgaagtacgcattcctgtagatcttgtaaagtctagttttgtgaaaaagtttggattgtgcaattatggaggaatccttatcagtcttattaatagtctagtagaaaataatttctttacaaaggatggaaaactggatgatacaggcaaaaaggaattggttttgacagatgtcgaaaaacgaattcttaataccatagataaatcatctcctttgtatatcgatattagtgatgttaaagtattggctgctagactaaaaagaagcgctacacaatttaactttaatggacatacatatcatctggaaaatgataaaatagaagatctcattaatcagttggttaaggacgaatccattcaactggatgaaaagagttctattaaagatagtatgtatgtcattcccgatgaacttatcgatgttctcaaaactagattgtttagatctcctcaagtcaaggataatattatttcgcgtactagattgtatgattattttactagagttactaagagagacgaatcgtcaatctatgtgattctaaaggatcctaggatcgctagcattttgtcactagaaactgttaaaatgggcgcctttatgtatacaaaacatagtatgttgacgaacgctatttcatctagagtcgatagatattctaaaaagtttcaagaatctttttacgaagatattgcagaatttgttaaagaaaatgagagagttaatgtatcgagagtggttgaatgtttgactgtgcctaatattactatatcaagtaatgctgaataa

SEQ ID NO: 30 - DNA, 1110 bp, Vaccinia virus
atgattgcgttattgatactatcgttaacgtgttcagtgtctacctatcgtctgcaaggatttaccaatgccggtatagtagcgtataaaaatattcaagatgataatattgtcttctcaccgtttggttattcgttttctatgtttatgtcgctattgcctgcatcaggtaatactagaatagaattattgaagactatggatttgagaaaaagagatctgggtccagcatttacagaattaatatcaggattagctaagctgaaaacatctaaatatacgtacactgatctaacttatcaaagtttcgtagataatactgtgtgcattaaaccgtcgtattatcaacaatatcatagattcggcctatatagattaaactttagacgagatgcggttaataaaattaattctatagtagaacgtagatccggtatgtctaatgtagtagattctaatatgctcgacaataatactctatgggcaatcattaatactatatattttaaaggtatatggcaatatccgtttgatatcactaaaacacgcaatgctagttttactaataagtacggtacgaaaacggttcccatgatgaacgtagttactaaattgcaaggaaatacaatcacaatcgatgacgaagaatatgatatggtacgccttccgtataaggatgctaatattagtatgtacctggcaataggtgataatatgacccatttcacagattctattacggctgcaaaattagactattggtcgtttcaattagggaataaagtgtacaatcttaaactccctaaattttctatcgaaaataagagggatattaagtcgatagccgaaatgatggctcctagtatgtttaatccagataatgcgtcgtttaaacatatgactagggacccattatatatttataaaatgtttcagaatgcaaagatagatgtcgacgaacaaggaactgtagcagaggcatctactattatggtagctacggcgagatcatctcctgaaaaactggaatttaatacaccatttgtgttcatcatcagacatgatattactggatttatattgtttatgggtaaggtggaatctccttaa

SEQ ID NO: 31 - DNA, 756 bp, Vaccinia virus
atgagtctactgctagaaaacctcatcgaagaagataccatattttttgcaggaagtatatctgagtatgatgatttacaaatggttattgccggcgcaaaatccaaatttccaagatctatgctttctatttttaatatagtacctagaacgatgtcaaaatatgagttggagttgattcataacgaaaatatcacaggagcaatgtttaccacaatgtataatataagaaacaatttgggtctaggagatgataaactaactattgaagccattgaaaactatttcttggatcctaacaatgaagttatgcctcttattattaataatacggatatgactgccgtcattcctaaaaaaagtggtaggagaaagaataagaacatggttatcttccgtcaaggatcatcacctatcttgtgtattttcgaaactcgtaaaaagattaatatttataaagaaaatatggaatccgcgtcgactgagtatacacctatcggagacaacaaggctttgatatctaaatatgcgggaattaatatcctaaatgtgtattctccttccacatccataagattgaatgccatttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattga

SEQ ID NO: 32 - DNA, 387 bp, Vaccinia virus
atggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatga

SEQ ID NO: 33 - DNA, 354 bp, Vaccinia virus
atgaggactctacttattagatatattctttggagaaatgacaacgatcaaacctattataatgatgattttaaaaagcttatgttgttggatgaattggtagatgacggcgatgtatgtacattgattaagaacatgagaatgacgctgtccgacggtccattgctagatagattgaatcaaccagttaataatatagaagacgctaagcgaatgatcgctattagtgccaaagtggctagagacattggtgaacgttcagaaattagatgggaagagtcattcaccatactctttaggatgattgaaacatattttgatgatctaatgattgatctatatggtgaaaaataa

SEQ ID NO: 34 - DNA, 327 bp, Vaccinia virus
atggccgaggaatttgtacaacaaaggttggccaataacaaagtgacaatttttgtcaagtatacatgtcctttttgtagaaatgcactggatattctaaataagtttagtttcaaaagaggagcgtatgaaattgtcgatattaaagaatttaaacccgaaaatgaattgcgtgactattttgaacaaattactggtggtagaactgttcctagaatcttttttgggaaaacttctattggtggatatagcgacctgttggaaatagacaacatggacgcattgggtgatattctatcatctattggggtattgagaacttgttga

SEQ ID NO: 35 - DNA, 1623 bp, Mycobacterium tuberculosis
atggccaagacaattgcgtacgacgaagaggcccgtcgcggcctcgagcggggcttgaacgccctcgccgatgcggtaaaggtgacattgggccccaagggccgcaacgtcgtcctggaaaagaagtggggtgcccccacgatcaccaacgatggtgtgtccatcgccaaggagatcgagctggaggatccgtacgagaagatcggcgccgagctggtcaaagaggtagccaagaagaccgatgacgtcgccggtgacggcaccacgacggccaccgtgctggcccaggcgttggttcgcgagggcctgcgcaacgtcgcggccggcgccaacccgctcggtctcaaacgcggcatcgaaaaggccgtggagaaggtcaccgagaccctgctcaagggcgccaaggaggtcgagaccaaggagcagattgcggccaccgcagcgatttcggcgggtgaccagtccatcggtgacctgatcgccgaggcgatggacaaggtgggcaacgagggcgtcatcaccgtcgaggagtccaacacctttgggctgcagctcgagctcaccgagggtatgcggttcgacaagggctacatctcggggtacttcgtgaccgacccggagcgtcaggaggcggtcctggaggacccctacatcctgctggtcagctccaaggtgtccactgtcaaggatctgctgccgctgctcgagaaggtcatcggagccggtaagccgctgctgatcatcgccgaggacgtcgagggcgaggcgctgtccaccctggtcgtcaacaagatccgcggcaccttcaagtcggtggcggtcaaggctcccggcttcggcgaccgccgcaaggcgatgctgcaggatatggccattctcaccggtggtcaggtgatcagcgaagaggtcggcctgacgctggagaacgccgacctgtcgctgctaggcaaggcccgcaaggtcgtggtcaccaaggacgagaccaccatcgtcgagggcgccggtgacaccgacgccatcgccggacgagtggcccagatccgccaggagatcgagaacagcgactccgactacgaccgtgagaagctgcaggagcggctggccaagctggccggtggtgtcgcggtgatcaaggccggtgccgccaccgaggtcgaactcaaggagcgcaagcaccgcatcgaggatgcggttcgcaatgccaaggccgccgtcgaggagggcatcgtcgccggtgggggtgtgacgctgttgcaagcggccccgaccctggacgagctgaagctcgaaggcgacgaggcgaccggcgccaacatcgtgaaggtggcgctggaggccccgctgaagcagatcgccttcaactccgggctggagccgggcgtggtggccgagaaggtgcgcaacctgccggctggccacggactgaacgctcagaccggtgtctacgaggatctgctcgctgccggcgttgctgacccggtcaaggtgacccgttcggcgctgcagaatgcggcgtccatcgcggggctgttcctgaccaccgaggccgtcgttgccgacaagccggaaaaggagaaggcttccgttcccggtggcggcgacatgggtggcatggatttctga

SEQ ID NO: 36 - DNA, 1287 bp, Mycobacterium tuberculosis
atgtctgtcgtcggcaccccgaagagcgcggagcagatccagcaggaatgggacacgaacccgcgctggaaggacgtcacccgcacctactccgccgaggacgtcgtcgccctccagggcagcgtggtcgaggagcacacgctggcccgccgcggtgcggaggtgctgtgggagcagctgcacgacctcgagtgggtcaacgcgctgggcgcgctgaccggcaacatggccgtccagcaggtgcgcgccggcctgaaggccatctacctgtcgggctggcaggtcgccggcgatgccaacctgtccgggcacacctaccccgaccagagcctgtatcccgccaactcggtgccgcaggtggtccgccggatcaacaacgcactgcagcgcgccgaccagatcgccaagatcgagggcgatacttcggtggagaactggctggcgccgattgtcgccgacggcgaggccggctttggcggcgcgctcaacgtctacgagctgcagaaagccctgatcgccgcgggcgttgcgggttcgcactgggaggaccagttggcctctgagaagaagtgcggccacctgggcggcaaggtgttgatcccgacccagcagcacatccgcactttgacgtctgctcggctcgcggccgatgtggctgatgttcccacggtggtgatcgcccgtaccgacgccgaggcggccacgctgatcacctccgacgtcgacgagcgcgaccagccgttcatcaccggcgagcgcacccgggaaggcttctaccgcaccaagaacggcatcgagccttgcatcgctcgggcgaaggcctacgccccgttcgccgacttgatctggatggagaccggtaccccggacctcgaggccgcccggcagttctccgaggcggtcaaggcggagtacccggaccagatgctggcctacaactgctcgccatcgttcaactggaaaaagcacctcgacgacgccaccatcgccaagttccagaaggagctggcagccatgggcttcaagttccagttcatcacgctggccggcttccatgcgctgaactactcgatgttcgatctggcctacggctacgcccagaaccagatgagcgcgtatgtcgaactgcaggaacgcgagttcgccgccgaagaacggggctacaccgcgaccaagcaccagcgcgaggtcggcgccggctacttcgaccggattgccaccaccgtggacccgaattcgtcgaccaccgcgttgaccggttccaccgaagagggccagttccactag

SEQ ID NO: 37 - DNA, 600 bp, Mycobacterium tuberculosis
atggctgaaaactcgaacattgatgacatcaaggctccgttgcttgccgcgcttggagcggccgacctggccttggccactgtcaacgagttgatcacgaacctgcgtgagcgtgcggaggagactcgtacggacacccgcagccgggtcgaggagagccgtgctcgcctgaccaagctgcaggaagatctgcccgagcagctcaccgagctgcgtgagaagttcaccgccgaggagctgcgtaaggccgccgagggctacctcgaggccgcgactagccggtacaacgagctggtcgagcgcggtgaggccgctctagagcggctgcgcagccagcagagcttcgaggaagtgtcggcgcgcgccgaaggctacgtggaccaggcggtggagttgacccaggaggcgttgggtacggtcgcatcgcagacccgcgcggtcggtgagcgtgccgccaagctggtcggcatcgagctgcctaagaaggctgctccggccaagaaggccgctccggccaagaaggccgctccggccaagaaggcggcggccaagaaggcgcccgcgaagaaggcggcggccaagaaggtcacccagaagtag

SEQ ID NO: 38 - DNA, 1647 bp, Mycobacterium tuberculosis
atggacgtcgctttgggggttgcggtcacggatcgggtcgcgcgtctggcgctggtcgactcggctgcgcccggcaccgtgatcgaccagttcgtgctcgatgtggccgagcacccggtcgaggtgttaaccgagaccgtggtgggcacggatcggtcattggccggcgaaaaccaccggctggtcgctacccggctgtgttggccggatcaggccaaagctgacgagctgcagcacgcactgcaggactccggggtccacgacgttgccgtgatatccgaggcgcaggccgccacggcgctggtcggggcggcacatgccggctctgccgtgctgttggtgggtgatgagacggcaaccttatcggtggttggtgacccggacgcgccgccgacgatggtggccgtcgcgccggtggcgggcgccgacgccacatcgaccgtcgataccctgatggcccggctcggcgaccaggccctcgccccgggggatgtcttcctggtgggtaggtccgccgagcacaccacggttcttgccgaccagctgcgcgcggcgtcgacgatgcgcgtgcagactcccgacgaccccacgttcgcgctggcccgtggcgcggcgatggcggccggcgccgctacgatggcgcacccggccctggtcgcggatgcgaccacttcgctcccccgggccgaggcggggcaatcgggttctgaaggcgagcagctggcgtactcgcaggccagcgattacgagctgcttccggtcgacgaatatgaggaacacgacgaatacggggcagccgcggatcgctcggcgccgttgagccgacggtcgctgctgatcggcaacgctgtcgtggcctttgcggtgatcggtttcgcctcgctggcggtggcggtggcggtcaccatccgaccgaccgcggcctcaaaaccggtagagggacaccaaaacgcccagccagggaagttcatgccgttgttgccgacgcaacagcaggcgccggtcccgccgcctccgcccgatgatcccaccgctggattccagggcggcaccattccggctgtacagaacgtggtgccgcggccgggtacctcacccggggtgggtgggacgccggcttcgcctgcgccggaagcgccggccgtgcccggtgttgtgcctgccccggtgccaatcccggtcccgatcatcattcccccgttcccgggttggcagcctggaatgccgaccatccccaccgcaccgccgacgacgccggtgaccacgtcggcgacgacgccgccgaccacgccgccgaccacgccggtgaccacgccgccaacgacgccgccgaccacgccggtgaccacgccgccaacgacgccgccgaccacgccggtgaccacgccaccaacgaccgtcgccccgacgaccgtcgccccgacgacggtcgctccgaccaccgtcgccccgaccacggtcgctccagccaccgccacgccgacgaccgtcgctccgcagccgacgcagcagcccacgcaacaaccaacccaacagatgccaacccagcagcagaccgtggccccgcagacggtggcgccggctccgcagccgccgtccggtggccgcaacggcagcggcgggggcgacttattcggcgggttctga

SEQ ID NO: 39 - DNA, 723 bp, Mycobacterium tuberculosis
atgccggccatgaccgcccgttcggtggtactcagcgtgctgctcggtgctcatcccgcgtgggccaccgcaagcgaattgatccagctgacagcggatttcggtatcaaggagacgacgttgcgggtcgcgctgacccgcatggtcggtgccggggatctggtccggtccgcggacggctaccggctctcggatcggttgctggcccgccagcgccgacaagatgaggccatgcgcccacggacccgcgcttggcacggaaactggcacatgctgattgtcaccagcatcggcaccgatgctcgtacccgggccgcactgcgaacctgcatgcaccacaagcgtttcggtgaattgcgggaaggggtgtggatgcggccggacaatctcgacctcgacttggagtccgacgttgcggcccgggttaggatgctgacggcccgcgacgaggcccccgccgacttggccgggcagctgtgggatctgtcggggtggaccgaggccggccaccggttgctcggcgacatggcagcggccaccgacatgcccgggcgatttgtggtggctgcggcgatggtgcgccacctgctcaccgatccgatgttgcccgctgaactgttgcccgccgactggccgggcgccgggttacgggcggcgtaccacgacttcgccactgcaatggcgaaacgacgcgatgcaactcaactcctggaggtgacatga

SEQ ID NO: 40 - DNA, 1191 bp, Mycobacterium tuberculosis
gtggcgaaggcgaagttccagcggaccaagccccacgtcaacatcgggaccatcggtcacgttgaccacggcaagaccaccctgaccgcggctatcaccaaggtcctgcacgacaaattccccgatctgaacgagacgaaggcattcgaccagatcgacaacgcccccgaggagcgtcagcgcggtatcaccatcaacatcgcgcacgtggagtaccagaccgacaagcggcactacgcacacgtcgacgcccctggccacgccgactacatcaagaacatgatcaccggcgccgcgcagatggacggtgcgatcctggtggtcgccgccaccgacggcccgatgccccagacccgcgagcacgttctgctggcgcgtcaagtgggtgtgccctacatcctggtagcgctgaacaaggccgacgcagtggacgacgaggagctgctcgaactcgtcgagatggaggtccgcgagctgctggctgcccaggaattcgacgaggacgccccggttgtgcgggtctcggcgctcaaggcgctcgagggtgacgcgaagtgggttgcctctgtcgaggaactgatgaacgcggtcgacgagtcgattccggacccggtccgcgagaccgacaagccgttcctgatgccggtcgaggacgtcttcaccattaccggccgcggaaccgtggtcaccggacgtgtggagcgcggcgtgatcaacgtgaacgaggaagttgagatcgtcggcattcgcccatcgaccaccaagaccaccgtcaccggtgtggagatgttccgcaagctgctcgaccagggccaggcgggcgacaacgttggtttgctgctgcggggcgtcaagcgcgaggacgtcgagcgtggccaggttgtcaccaagcccggcaccaccacgccgcacaccgagttcgaaggccaggtctacatcctgtccaaggacgagggcggccggcacacgccgttcttcaacaactaccgtccgcagttctacttccgcaccaccgacgtgaccggtgtggtgacactgccggagggcaccgagatggtgatgcccggtgacaacaccaacatctcggtgaagttgatccagcccgtcgccatggacgaaggtctgcgtttcgcgatccgcgagggtggccgcaccgtgggcgccggccgggtcaccaagatcatcaagtag

SEQ ID NO: 41 - DNA, 798 bp, Mycobacterium tuberculosis
atgaacaatctctaccgcgatttggcaccggtcaccgaagccgcttgggcggaaatcgaattggaggcggcgcggacgttcaagcgacacatcgccgggcgccgggtggtcgatgtcagtgatcccggggggcccgtcaccgcggcggtcagcaccggccggctgatcgatgttaaggcaccaaccaacggcgtgatcgcccacctgcgggccagcaaaccccttgtccggctacgggttccgtttaccctgtcgcgcaacgagatcgacgacgtggaacgtggctctaaggactccgattgggaaccggtaaaggaggcggccaagaagctggccttcgtcgaggaccgcacaatattcgaaggctacagcgccgcatcaatcgaagggatccgcagcgcgagttcgaacccggcgctgacgttgcccgaggatccccgtgaaatccctgatgtcatctcccaggcattgtccgaactgcggttggccggtgtggacggaccgtattcggtgttgctctctgctgacgtctacaccaaggttagcgagacttccgatcacggctatcccatccgtgagcatctgaaccggctggtggacggggacatcatttgggccccggccatcgacggcgcgttcgtgctgaccactcgaggcggcgacttcgacctacagctgggcaccgacgttgcaatcgggtacgccagccacgacacggacaccgtgcgcctctacctgcaggagacgctgacgttcctttgctacaccgccgaggcgtcggtcgcgctcagccactaa

SEQ ID NO: 42 - DNA, 300 bp, Mycobacterium tuberculosis
atgtcttttgtgaccatccagccggtggtcttggcagccgcgacgggggacttgccgacgatcggtaccgccgtgagtgctcggaacacagccgtctgtgccccgacgacgggggtgttaccccctgctgccaatgacgtgtcggtcctgacggcggcccggttcaccgcgcacaccaagcactaccgagtggtgagtaagccggccgcgctggtccatggcatgttcgtggccctcccggcggccaccgccgatgcgtatgcgaccaccgaggccgtcaatgtggtcgcgaccggttaa

SEQ ID NO: 43 - DNA, 1125 bp, Mycobacterium tuberculosis
gtgaaaattcgtttgcatacgctgttggccgtgttgaccgctgcgccgctgctgctagcagcggcgggctgtggctcgaaaccaccgagcggttcgcctgaaacgggcgccggcgccggtactgtcgcgactacccccgcgtcgtcgccggtgacgttggcggagaccggtagcacgctgctctacccgctgttcaacctgtggggtccggcctttcacgagaggtatccgaacgtcacgatcaccgctcagggcaccggttctggtgccgggatcgcgcaggccgccgccgggacggtcaacattggggcctccgacgcctatctgtcggaaggtgatatggccgcgcacaaggggctgatgaacatcgcgctagccatctccgctcagcaggtcaactacaacctgcccggagtgagcgagcacctcaagctgaacggaaaagtcctggcggccatgtaccagggcaccatcaaaacctgggacgacccgcagatcgctgcgctcaaccccggcgtgaacctgcccggcaccgcggtagttccgctgcaccgctccgacgggtccggtgacaccttcttgttcacccagtacctgtccaagcaagatcccgagggctggggcaagtcgcccggcttcggcaccaccgtcgacttcccggcggtgccgggtgcgctgggtgagaacggcaacggcggcatggtgaccggttgcgccgagacaccgggctgcgtggcctatatcggcatcagcttcctcgaccaggccagtcaacggggactcggcgaggcccaactaggcaatagctctggcaatttcttgttgcccgacgcgcaaagcattcaggccgcggcggctggcttcgcatcgaaaaccccggcgaaccaggcgatttcgatgatcgacgggcccgccccggacggctacccgatcatcaactacgagtacgccatcgtcaacaaccggcaaaaggacgccgccaccgcgcagaccttgcaggcatttctgcactgggcgatcaccgacggcaacaaggcctcgttcctcgaccaggttcatttccagccgctgccgcccgcggtggtgaagttgtctgacgcgttgatcgcgacgatttccagctag

SEQ ID NO: 44 - DNA, 1272 bp, Mycobacterium tuberculosis
atggatttcgggttgttaccgccggagatcaactcaggcaggatgtatacggggccggggccggggcccatgctggccgccgcgacagcctgggacgggctggctgttgagctgcacgcaacagcggctggctacgcctcggagctatcggctttgaccggggcatggagcggtccttcgtcgacgtccatggcatctgcagccgcaccctatgtggcatggatgagcgccaccgcagtgcatgccgagctggcgggcgcgcaagccaggttggcgatagctgcctatgaagctgcgttcgctgccaccgtgcctccgccggtgatcgccgctaatcgtgcccaactgatggtgttgatcgcgacgaacatcttcgggcagaacacgccggcgatcatgatgactgaggcccaatacatggaaatgtgggcgcaggatgccgccgcgatgtacgggtacgccggctcgtcagcgaccgcctcgcgaatgacagcgttcactgagccgccgcaaaccactaaccatggtcagttgggggcccagtcctccgccgtcgcacaaaccgccgccaccgcggccggcggcaacctgcaatcggcattcccgcagctgctctccgcggttccccgcgccctgcaaggcctggcattgccgaccgcatcacagtcggcatcggcgacgccgcagtgggttaccgacctggggaacctgtccaccttcctgggcggggcggtcaccggcccgtacacctttcccggggtattgcctccctccggggtgccatacctgttaggcattcagagcgtcttggtaacccaaaacgggcagggggtaagcgccttgcttggcaagatcggggggaaaccaatcaccggagcgttggctccgctggccgaatttgctttgcatacaccaattttgggttcggagggcttgggtggtggatcggtttccgcgggtattggccgggcaggcttggtcggaaagctatcggtgcctcagggctggacggtggccgccccggagatcccatcgccggcggcggcgttgcaggcgacgcgcctggccgccgcgccgattgcggccaccgacggcgcgggtgcgttgctcggtggcatggcgctgtcgggcttggctggccgcgctgccgccggttctaccggccaccccatcggcagcgccgcagcacccgccgtcggtgccgctgccgctgccgtcgaggacctggccaccgaagccaacatcttcgtgataccggccatggacgactag

SEQ ID NO: 45 - DNA, 978 bp, Mycobacterium tuberculosis
atgcatcaggtggaccccaacttgacacgtcgcaagggacgattggcggcactggctatcgcggcgatggccagcgccagcctggtgaccgttgcggtgcccgcgaccgccaacgccgatccggagccagcgcccccggtacccacaacggccgcctcgccgccgtcgaccgctgcagcgccacccgcaccggcgacacctgttgcccccccaccaccggccgccgccaacacgccgaatgcccagccgggcgatcccaacgcagcacctccgccggccgacccgaacgcaccgccgccacctgtcattgccccaaacgcaccccaacctgtccggatcgacaacccggttggaggattcagcttcgcgctgcctgctggctgggtggagtctgacgccgcccacttcgactacggttcagcactcctcagcaaaaccaccggggacccgccatttcccggacagccgccgccggtggccaatgacacccgtatcgtgctcggccggctagaccaaaagctttacgccagcgccgaagccaccgactccaaggccgcggcccggttgggctcggacatgggtgagttctatatgccctacccgggcacccggatcaaccaggaaaccgtctcgctcgacgccaacggggtgtctggaagcgcgtcgtattacgaagtcaagttcagcgatccgagtaagccgaacggccagatctggacgggcgtaatcggctcgcccgcggcgaacgcaccggacgccgggccccctcagcgctggtttgtggtatggctcgggaccgccaacaacccggtggacaagggcgcggccaaggcgctggccgaatcgatccggcctttggtcgccccgccgccggcgccggcaccggctcctgcagagcccgctccggcgccggcgccggccggggaagtcgctcctaccccgacgacaccgacaccgcagcggaccttaccggcctga

SEQ ID NO: 46 - DNA, 480 bp, Mycobacterium tuberculosis
atgaagctcaccacaatgatcaagacggcagtagcggtcgtggccatggcggccatcgcgacctttgcggcaccggtcgcgttggctgcctatcccatcaccggaaaacttggcagtgagctaacgatgaccgacaccgttggccaagtcgtgctcggctggaaggtcagtgatctcaaatccagcacggcagtcatccccggctatccggtggccggccaggtctgggaggccactgccacggtcaatgcgattcgcggcagcgtcacgcccgcggtctcgcagttcaatgcccgcaccgccgacggcatcaactaccgggtgctgtggcaagccgcgggccccgacaccattagcggagccactatcccccaaggcgaacaatcgaccggcaaaatctacttcgatgtcaccggcccatcgccaaccatcgtcgcgatgaacaacggcatggaggatctgctgatttgggagccgtag

SEQ ID NO: 47 - DNA, 687 bp, Mycobacterium tuberculosis
gtgcgcatcaagatcttcatgctggtcacggctgtcgttttgctctgttgttcgggtgtggccacggccgcgcccaagacctactgcgaggagttgaaaggcaccgataccggccaggcgtgccagattcaaatgtccgacccggcctacaacatcaacatcagcctgcccagttactaccccgaccagaagtcgctggaaaattacatcgcccagacgcgcgacaagttcctcagcgcggccacatcgtccactccacgcgaagccccctacgaattgaatatcacctcggccacataccagtccgcgataccgccgcgtggtacgcaggccgtggtgctcaaggtctaccagaacgccggcggcacgcacccaacgaccacgtacaaggccttcgattgggaccaggcctatcgcaagccaatcacctatgacacgctgtggcaggctgacaccgatccgctgccagtcgtcttccccattgtgcaaggtgaactgagcaagcagaccggacaacaggtatcgatagcgccgaatgccggcttggacccggtgaattatcagaacttcgcagtcacgaacgacggggtgattttcttcttcaacccgggggagttgctgcccgaagcagccggcccaacccaggtattggtcccacgttccgcgatcgactcgatgctggcctag

SEQ ID NO: 48 - DNA, 654 bp, Mycobacterium tuberculosis
atgactccacgcagccttgttcgcatcgttggtgtcgtggttgcgacgaccttggcgctggtgagcgcacccgccggcggtcgtgccgcgcatgcggatccgtgttcggacatcgcggtcgttttcgctcgcggcacgcatcaggcttctggtcttggcgacgtcggtgaggcgttcgtcgactcgcttacctcgcaagttggcgggcggtcgattggggtctacgcggtgaactacccagcaagcgacgactaccgcgcgagcgcgtcaaacggttccgatgatgcgagcgcccacatccagcgcaccgtcgccagctgcccgaacaccaggattgtgcttggtggctattcgcagggtgcgacggtcatcgatttgtccacctcggcgatgccgcccgcggtggcagatcatgtcgccgctgtcgcccttttcggcgagccatccagtggtttctccagcatgttgtggggcggcgggtcgttgccgacaatcggtccgctgtatagctctaagaccataaacttgtgtgctcccgacgatccaatatgcaccggaggcggcaatattatggcgcatgtttcgtatgttcagtcggggatgacaagccaggcggcgacattcgcggcgaacaggctcgatcacgccggatga

SEQ ID NO: 49 - DNA, 345 bp, Mycobacterium tuberculosis
gtgacctatgtgatcggtagtgagtgcgtggatgtgatggacaagtcctgtgtgcaggagtgtccggtcgactgtatctatgagggcgcccgaatgctctacatcaaccccgacgagtgcgtggattgtggtgcgtgcaaaccggcctgccgcgtcgaggcgatctactgggaaggcgatctacccgacgatcaacaccagcatctgggggacaacgccgcctttttccaccaagtcctgccgggccgagtggctccgctgggttcgccgggtggtgccgcagcggtgggcccgatcggagtcgacacgcctctggtcgcggctatcccggtggagtgcccttag

SEQ ID NO: 50 - DNA, 435 bp, Mycobacterium tuberculosis
atggccaccacccttcccgttcagcgccacccgcggtccctcttccccgagttttctgagctgttcgcggccttcccgtcattcgccggactccggcccaccttcgacacccggttgatgcggctggaagacgagatgaaagaggggcgctacgaggtacgcgcggagcttcccggggtcgaccccgacaaggacgtcgacattatggtccgcgatggtcagctgaccatcaaggccgagcgcaccgagcagaaggacttcgacggtcgctcggaattcgcgtacggttccttcgttcgcacggtgtcgctgccggtaggtgctgacgaggacgacattaaggccacctacgacaagggcattcttactgtgtcggtggcggtttcggaagggaagccaaccgaaaagcacattcagatccggtccaccaactga

SEQ ID NO: 51 - DNA, 1158 bp, Mycobacterium tuberculosis
ttgaggctcgaccagaggtggttgatcgcgcgtgtaatcatgcggtccgccataggtttctttgcgagcttcaccgtctcctccggcgtcctggccgcgaatgtgctggctgatccggccgacgacgcgctggccaagctcaacgagttatcccggcaggccgagcagaccaccgaggcgctgcacagtgcgcagctggatctcaacgaaaagctcgctgcccagcgggccgccgaccagaagcttgcggacaacagaacggccttggatgctgcgagagcacgcttggcgacttttcagacggcggtgaacaaggtcgcggccgctacctacatgggtggtcgtacccacggcatggatgcgatcctgacggcggagtccccgcaactgttgatcgatcggctatcggtacagcgggtgatggcgcatcaaatgtccacgcagatggcccgtttcaaggccgctggagaacaggccgtcaaggccgagcaggctgcagccaaatcggcggccgatgccaggtccgcggccgagcaagctgccgcggtacgagcgaatctgcagcacaaacagagccagctgcaggtgcagattgccgtcgtcaagtcgcaatacgtcgcgttgacgccggaggagcgcacggccctcgctgatccaggaccggtcccggcggttgctgcgatcgcccccggggccccacctgcggcgttgccgcccggtgcgccgcctggcgacggcccggcgcctggcgtggcgccgccgcctggtgggatgcccggattgcctttcgtgcagcccgacggcgctggcggcgaccgtacggccgttgtccaagcggcgttgacgcaggtcggcgcgccctacgcgtggggtggtgccgcgcccggcgggttcgactgctcaggcttggtgatgtgggcgttccagcaggctggtatcgcgttaccgcactccagccaggcgctggctcacggtggtcagccggtcgcgttgtcggatctgcagcccggcgacgtgttgaccttctattccgacgcgtcacacgcaggcatctacatcggtgatggtcttatggttcattcctccacctacggtgttccggtgcgggtggtgccgatggactcgtcggggccgatctacgacgcccgccgttactga

SEQ ID NO: 52 - DNA, 1437 bp, Mycobacterium tuberculosis
gtgacggaaaagacgcccgacgacgtcttcaaacttgccaaggacgagaaggtcgaatatgtcgacgtccggttctgtgacctgcctggcatcatgcagcacttcacgattccggcttcggcctttgacaagagcgtgtttgacgacggcttggcctttgacggctcgtcgattcgcgggttccagtcgatccacgaatccgacatgttgcttcttcccgatcccgagacggcgcgcatcgacccgttccgcgcggccaagacgctgaatatcaacttctttgtgcacgacccgttcaccctggagccgtactcccgcgacccgcgcaacatcgcccgcaaggccgagaactacctgatcagcactggcatcgccgacaccgcatacttcggcgccgaggccgagttctacattttcgattcggtgagcttcgactcgcgcgccaacggctccttctacgaggtggacgccatctcggggtggtggaacaccggcgcggcgaccgaggccgacggcagtcccaaccggggctacaaggtccgccacaagggcgggtatttcccagtggcccccaacgaccaatacgtcgacctgcgcgacaagatgctgaccaacctgatcaactccggcttcatcctggagaagggccaccacgaggtgggcagcggcggacaggccgagatcaactaccagttcaattcgctgctgcacgccgccgacgacatgcagttgtacaagtacatcatcaagaacaccgcctggcagaacggcaaaacggtcacgttcatgcccaagccgctgttcggcgacaacgggtccggcatgcactgtcatcagtcgctgtggaaggacggggccccgctgatgtacgacgagacgggttatgccggtctgtcggacacggcccgtcattacatcggcggcctgttacaccacgcgccgtcgctgctggccttcaccaacccgacggtgaactcctacaagcggctggttcccggttacgaggccccgatcaacctggtctatagccagcgcaaccggtcggcatgcgtgcgcatcccgatcaccggcagcaacccgaaggccaagcggctggagttccgaagccccgactcgtcgggcaacccgtatctggcgttctcggccatgctgatggcaggcctggacggtatcaagaacaagatcgagccgcaggcgcccgtcgacaaggatctctacgagctgccgccggaagaggccgcgagtatcccgcagactccgacccagctgtcagatgtgatcgaccgtctcgaggccgaccacgaatacctcaccgaaggaggggtgttcacaaacgacctgatcgagacgtggatcagtttcaagcgcgaaaacgagatcgagccggtcaacatccggccgcatccctacgaattcgcgctgtactacgacgtttaa

SEQ ID NO: 53 - DNA, 507 bp, Mycobacterium tuberculosis
atgaagatggtgaaatcgatcgccgcaggtctgaccgccgcggctgcaatcggcgccgctgcggccggtgtgacttcgatcatggctggcggcccggtcgtataccagatgcagccggtcgtcttcggcgcgccactgccgttggacccggcatccgcccctgacgtcccgaccgccgcccagttgaccagcctgctcaacagcctcgccgatcccaacgtgtcgtttgcgaacaagggcagtctggtcgagggcggcatcgggggcaccgaggcgcgcatcgccgaccacaagctgaagaaggccgccgagcacggggatctgccgctgtcgttcagcgtgacgaacatccagccggcggccgccggttcggccaccgccgacgtttccgtctcgggtccgaagctctcgtcgccggtcacgcagaacgtcacgttcgtgaatcaaggcggctggatgctgtcacgcgcatcggcgatggagttgctgcaggccgcagggaactga

SEQ ID NO: 54 - DNA, 465 bp, Mycobacterium tuberculosis
atgacaccgggtttgcttactactgcgggtgctggccgaccacgtgacaggtgcgccaggatcgtatgcacggtgttcatcgaaaccgccgttgtcgcgaccatgtttgtcgcgttgttgggtctgtccaccatcagctcgaaagccgacgacatcgattgggacgccatcgcgcaatgcgaatccggcggcaattgggcggccaacaccggtaacgggttatacggtggtctgcagatcagccaggcgacgtgggattccaacggtggtgtcgggtcgccggcggccgcgagtccccagcaacagatcgaggtcgcagacaacattatgaaaacccaaggcccgggtgcgtggccgaaatgtagttcttgtagtcagggagacgcaccgctgggctcgctcacccacatcctgacgttcctcgcggccgagactggaggttgttcggggagcagggacgattga

SEQ ID NO: 55 - DNA, 372 bp, Mycobacterium tuberculosis
atgaccgaccggtcgcgtgagccggctgacccgtggaagggattcagcgcggtgatggcggcgacgctgatcctcgaggcgatcgtggtgctgctggcaataccggtagtggacgcggtcggcggtgggctgcgtccggcctcgctgggctatttggtcggtctggccgtgctgttgatactgctgaccgggctgcagcgcagaccctgggcaatctgggtgaacctgggcgcacaaccggtgctggttgccggcttcgccgtgtacccgggtgtgggtttcattggcgtgctgttcgccgcgttgtgggtcctgatcgcgtatttgcgtgccgaggtgcggcggcgtcgggattaccgggtgtcgcaatga

SEQ ID NO: 56 - DNA, 813 bp, Mycobacterium tuberculosis
atggccaatccgttcgttaaagcctggaagtacctcatggcgctgttcagctcgaagatcgacgagcatgccgaccccaaggtgcagattcaacaggccattgaggaagcacagcgcacccaccaagcgctgactcaacaggcggcgcaagtgatcggtaaccagcgtcaattggagatgcgactcaaccgacagctggcggacatcgaaaagcttcaggtcaatgtgcgccaagccctgacgctggccgaccaggccaccgccgccggagacgctgccaaggccaccgaatacaacaacgccgccgaggcgttcgcagcccagctggtgaccgccgagcagagcgtcgaagacctcaagacgctgcatgaccaggcgcttagcgccgcagctcaggccaagaaggccgtcgaacgaaatgcgatggtgctgcagcagaagatcgccgagcgaaccaagctgctcagccagctcgagcaggcgaagatgcaggagcaggtcagcgcatcgttgcggtcgatgagtgagctcgccgcgccaggcaacacgccgagcctcgacgaggtgcgcgacaagatcgagcgtcgctacgccaacgcgatcggttcggctgaacttgccgagagttcggtgcagggccggatgctcgaggtggagcaggccgggatccagatggccggtcattcacggttggaacagatccgcgcatcgatgcgcggtgaagcgttgccggccggcgggaccacggctacccccagaccggccaccgagacttctggcggggctattgccgagcagccctacggtcagtag

SEQ ID NO: 57 - DNA, 663 bp, Mycobacterium tuberculosis
atgatcaacgttcaggccaaaccggccgcagcagcgagcctcgcagccatcgcgattgcgttcttagcgggttgttcgagcaccaaacccgtgtcgcaagacaccagcccgaaaccggcgaccagcccggcggcgcccgttaccacggcggcaatggctgaccccgcagcggacctgattggtcgtgggtgcgcgcaatacgcggcgcaaaatcccaccggtcccggatcggtggccggaatggcgcaagacccggtcgctaccgcggcttccaacaacccgatgctcagtaccctgacctcggctctgtcgggcaagctgaacccggatgtgaatctggtcgacaccctcaacggcggcgagtacaccgttttcgcccccaccaacgccgcattcgacaagctgccggcggccactatcgatcaactcaagactgacgccaagctgctcagcagcatcctgacctaccacgtgatagccggccaggcgagtccgagcaggatcgacggcacccatcagaccctgcaaggtgccgacctgacggtgataggcgcccgcgacgacctcatggtcaacaacgccggtttggtatgtggcggagttcacaccgccaacgcgacggtgtacatgatcgatacggtgctgatgcccccggcacagtaa

SEQ ID NO: 58 - DNA, 582 bp, Mycobacterium tuberculosis
atgaaggtaaagaacacaattgcggcaaccagtttcgcggcggccggcctggcggctctggcggtggctgtctcaccgccggcggccgcaggcgatctggtgggcccgggctgcgcggaatacgcggcagccaatcccactgggccggcctcggtgcagggaatgtcgcaggacccggtcgcggtggcggcctcgaacaatccggagttgacaacgctgacggctgcactgtcgggccagctcaatccgcaagtaaacctggtggacaccctcaacagcggtcagtacacggtgttcgcaccgaccaacgcggcatttagcaagctgccggcatccacgatcgacgagctcaagaccaattcgtcactgctgaccagcatcctgacctaccacgtagtggccggccaaaccagcccggccaacgtcgtcggcacccgtcagaccctccagggcgccagcgtgacggtgaccggtcagggtaacagcctcaaggtcggtaacgccgacgtcgtctgtggtggggtgtctaccgccaacgcgacggtgtacatgattgacagcgtgctaatgcctccggcgtaa

SEQ ID NO: 59 - DNA, 2157 bp, Mycobacterium tuberculosis
atgaccctggaagtggtatcggacgcggccggacgcatgcgggtcaaagtcgactgggtccgttgcgattcccggcgcgcggtcgcggtcgaagaggccgttgccaagcagaacggtgtgcgcgtcgtgcacgcctacccgcgcaccgggtccgtggtcgtgtggtattcacccagacgcgccgaccgcgcggcggtgctggcggcgatcaagggcgccgcgcacgtcgccgccgaactgatccccgcgcgtgcgccgcactcggccgagatccgcaacaccgacgtgctccggatggtcatcggcggggtggcactggccttgctcggggtgcgccgctacgtgttcgcgcggccaccgctgctcggaaccaccgggcggacggtggccaccggtgtcaccattttcaccgggtatccgttcctgcgtggcgcgctgcgctcgctgcgctccggaaaggccggcaccgatgccctggtctccgcggcgacggtggcaagcctcatcctgcgcgagaacgtggtcgcactcaccgtcctgtggttgctcaacatcggtgagtacctgcaggatctgacgctgcggcggacccggcgggccatctcggagctgctgcgcggcaaccaggacacggcctgggtgcgcctcaccgatccttctgcaggctccgacgcggccaccgaaatccaggtcccgatcgacaccgtgcagatcggtgacgaggtggtggtccacgagcacgtcgcgataccggtcgacggtgaggtggtcgacggcgaagcgatcgtcaatcagtccgcgatcaccggggaaaacctgccggtcagcgtcgtggtcggaacgcgcgtgcacgccggttcggtcgtggtgcgcggacgcgtggtggtgcgcgcccacgcggtaggcaaccaaaccaccatcggtcgcatcattagcagggtcgaagaggctcagctcgaccgggcacccatccagacggtgggcgagaacttctcccgccgcttcgttcccacctcgttcatcgtctcggccatcgcgttgctgatcaccggcgacgtgcggcgcgcgatgaccatgttgttgatcgcatgcccgtgcgcggtgggactgtccaccccgaccgcgatcagcgcagcgatcggcaacggcgcgcgccgtggcatcctgatcaagggcggatcccacctcgagcaggcgggccgcgtcgacgccatcgtgttcgacaagaccgggacgttgaccgtgggccgccccgtggtcaccaatatcgttgccatgcataaagattgggagcccgagcaagtgctggcctatgccgccagctcggagatccactcacgtcatccgctggccgaggcggtgatccgctcgacggaggaacgccgcatcagcatcccaccacacgaggagtgcgaggtgctggtcggcctgggcatgcggacctgggccgacggtcggaccctgctgctgggcagtccgtcgttgctgcgcgccgaaaaagttcgggtgtccaagaaggcgtcggagtgggtcgacaagctgcgccgccaggcggagaccccgctgctgctcgcggtggacggcacgctggtcggcctgatcagcctgcgcgacgaggtgcgtccggaggcggcccaggtgctgacgaagctgcgggccaatgggattcgccggatcgtcatgctcaccggcgaccacccggagatcgcccaggttgtcgccgacgaactggggattgatgagtggcgcgccgaggtcatgccggaggacaagctcgcggcggtgcgcgagctgcaggacgacggctacgtcgtcgggatggtcggcgacggcatcaacgacgccccggcgctggccgccgccgatatcgggatcgccatgggccttgccggaaccgacgtcgccgtcgagaccgccgatgtcgcgctggccaacgacgacctgcaccgcctgctcgacgttggggacctgggcgagcgggcagtggatgtaatccggcagaactacggcatgtccatcgccgtcaacgcggccgggctgctgatcggcgcgggcggtgcgctctcgccggtgctggcggcgatcctgcacaacgcgtcgtcggtggcggtggtggccaacagttcccggttgatccgctaccgcctggaccgctag

SEQ ID NO: 60 - DNA, 1218 bp, Mycobacterium tuberculosis
atggccttcttacgttcggtatcgtgcctggcagcagccgtgtttgcggtaggcaccggaattggtctacctaccgcggccggcgaacccaatgccgcaccggcggcgtgcccgtacaaggtgtccaccccacccgccgtggactcgtcggaggttcccgcggccggtgaacccccactgccgctggtggtaccccccaccccggtcggcggcaacgcgctgggcggctgcggcatcatcaccgcccctggcagcgcgccagcgcccggcgacgtctcagccgaggcctggctggtggcggacctggacagcggcgcggtgatcgccgcccgggatccgcacggccggcaccgcccggccagcgtcatcaaggtgctggtggcgatggcgtccatcaacacgctcaccctcaacaagtcggtcgccggaaccgccgacgacgcggcggtcgagggcaccaaagtcggggtgaacaccggtggcacctacaccgtcaaccagctgctgcacgggctgctgatgcactccggcaacgacgctgcgtacgcgctggccaggcagctcggcggcatgccggccgcgctggagaaaatcaatctgctggccgccaagctgggcggccgggacacccgagtggccacgccgtccggactggacgggcccggcatgagcacgtcggcctatgacatcggcctgttctaccggtacgcgtggcagaacccggtcttcgccgacatcgtcgcgacccgcaccttcgacttcccggggcacggcgaccatccaggctacgagttggagaacgacaaccagctgctctacaactatccgggcgcgctcggcggcaagaccggctataccgacgacgcggggcagaccttcgtgggcgcggccaaccgcgacggccggcggctgatgacggtgctgctgcacgggacccggcagccgatcccgccgtgggagcaggcggcgcacctgctcgactacgggttcaacaccccggcaggcacccagatcgggacactgatcgaacccgacccgtcgctgatgtccaccgaccgcaatcccgccgaccggcaacgagtcgacccccaggccgcggcgcggatatcggccgccgacgcccttccggtgcgggttggcgtggccgtcatcggcgccctgatcgtgttcgggttgatcatggtcgcgcgggcgatgaaccgccggccgcagcactag

SEQ ID NO: 61 - DNA, 846 bp, Mycobacterium tuberculosis
atgttcaccggcatcgctagccatgccggcgccctgggtgccgccttagtggtgctgatcggcgccgcaattctgcacgacggcccagcagcggccgacccaaaccaagacgatcggtttctggcgctgctcgagaaaaaggaaatccccgccgtcgcgaatgtgcctcgcgtcatcgacgcggcccacaaagtgtgtcgcaaactcgatggcggcatgccggtgaacgacattgtggacgggttacgcaacgatgcctacaacatagacccggtcatgcgcctctaccctgtccgcctcacgacgaccatgacccgatttatcagtgcggcagtggagatctactgcccgaaccatcacagcaagatggcgttcgccatggccaatttcgagccgggatcgaatgaaccgacgcatcgcgttgcggcgtccacgcgcagcgcggtcaactcgggaagcgacctgcgggcgtcggtgtcggacatgaccatcatgtcgccgggatggcgggaaccgacgggtgcgatgcttgcctcggtgctcggagcggttcgcgcgggggatcccctgataccgaatccgccgccgattccggtaccgccgccggcggcgcagaccctgattccacccccgccgatcgtggcaccgccgccaccgcgaccagcgccgccgcaacagccgccgcccccgccgccagaggttgagccgcctgctggtgttccgcagtccgggggcgctgccggcagtggcggcgccggcagcggtggtggcggcggtggtgacggaccggtagagccgtcgcctgcacgacccatgccgccgggctttatcaggctcgcgccgtga

SEQ ID NO: 62 - DNA, 303 bp, Mycobacterium tuberculosis
gtggcgaaggtgaacatcaagccactcgaggacaagattctcgtgcaggccaacgaggccgagaccacgaccgcgtccggtctggtcattcctgacaccgccaaggagaagccgcaggagggcaccgtcgttgccgtcggccctggccggtgggacgaggacggcgagaagcggatcccgctggacgttgcggagggtgacaccgtcatctacagcaagtacggcggcaccgagatcaagtacaacggcgaggaatacctgatcctgtcggcacgcgacgtgctggccgtcgtttccaagtag

SEQ ID NO: 63 - DNA, 480 bp, Mycobacterium tuberculosis
gtgaagcgtggactgacggtcgcggtagccggagccgccattctggtcgcaggtctttccggatgttcaagcaacaagtcgactacaggaagcggtgagaccacgaccgcggcaggcacgacggcaagccccggcgccgcctccgggccgaaggtcgtcatcgacggtaaggaccagaacgtcaccggctccgtggtgtgcacaaccgcggccggcaatgtcaacatcgcgatcggcggggcggcgaccggcattgccgccgtgctcaccgacggcaaccctccggaggtgaagtccgttgggctcggtaacgtcaacggcgtcacgctgggatacacgtcgggcaccggacagggtaacgcctcggcaaccaaggacggcagccactacaagatcactgggaccgctaccggggtcgacatggccaacccgatgtcaccggtgaacaagtcgttcgaaatcgaggtgacctgttcctaa

SEQ ID NO: 64 - DNA, 900 bp, Mycobacterium tuberculosis
atgaagggtcggtcggcgctgctgcgggcgctctggattgccgcactgtcattcgggttgggcggtgtcgcggtagccgcggaacccaccgccaaggccgccccatacgagaacctgatggtgccgtcgccctcgatgggccgggacatcccggtggccttcctagccggtgggccgcacgcggtgtatctgctggacgccttcaacgccggcccggatgtcagtaactgggtcaccgcgggtaacgcgatgaacacgttggcgggcaaggggatttcggtggtggcaccggccggtggtgcgtacagcatgtacaccaactgggagcaggatggcagcaagcagtgggacaccttcttgtccgctgagctgcccgactggctggccgctaaccggggcttggcccccggtggccatgcggccgttggcgccgctcagggcggttacggggcgatggcgctggcggccttccaccccgaccgcttcggcttcgctggctcgatgtcgggctttttgtacccgtcgaacaccaccaccaacggtgcgatcgcggcgggcatgcagcaattcggcggtgtggacaccaacggaatgtggggagcaccacagctgggtcggtggaagtggcacgacccgtgggtgcatgccagcctgctggcgcaaaacaacacccgggtgtgggtgtggagcccgaccaacccgggagccagcgatcccgccgccatgatcggccaagccgccgaggcgatgggtaacagccgcatgttctacaaccagtatcgcagcgtcggcgggcacaacggacacttcgacttcccagccagcggtgacaacggctggggctcgtgggcgccccagctgggcgctatgtcgggcgatatcgtcggtgcgatccgctaa

SEQ ID NO: 65 - DNA, 612 bp, Mycobacterium tuberculosis
ttgtcggtggtgtgctgtaggaacagatggatgaatttggcggtgtgggcggagcgcaacggtgttgcgtgggtgatcgcgtatcgctggtttcgagccgggctgttgccggttccggcgcagcgagtgggtcggctcattctggtgaacgatccggcagtcgaggagtctgggcgcgggcggacgttggtgtacgcgcgggtatcgtcagcggatcagaggtccgatctggatcggcgggtcgcgcgggtgaccgcgtgggccacatcgcaacatctctctgtcgacaaggtggtggccgagggtggttgggcgttgaatggacatcgccgtaagttttttgcgctgctgggtgatccggtggtgacgcggatcgtggtggagcaccgggatcggttctgctggtttggctctgagtacgtcgaggccgctcttgtcgcccagggccgggaattggtggtggtcgacttggctgaggttgatgacgacctggtgggcgatatgaccgagatcctgacctcgatgtgtgctcggctctatggtgaacgcgctgcacagaacggggccaagcgtgcccttgctgctgcggtcggggacgcggaggcagcttga

SEQ ID NO: 66 - DNA, 624 bp, Mycobacterium tuberculosis
gtggccgaatacaccttgccagacctggactgggactacggagcactggaaccgcacatctcgggtcagatcaacgagcttcaccacagcaagcaccacgccacctacgtaaagggcgccaatgacgccgtcgccaaactcgaagaggcgcgcgccaaggaagatcactcagcgatcttgctgaacgaaaagaatctagctttcaacctcgccggccacgtcaatcacaccatctggtggaagaacctgtcgcctaacggtggtgacaagcccaccggcgaactcgccgcagccatcgccgacgcgttcggttcgttcgacaagttccgtgcgcagttccacgcggccgctaccaccgtgcaggggtcgggctgggcggcactgggctgggacacactcggcaacaagctgctgatattccaggtttacgaccaccagacgaacttcccgctaggcattgttccgctgctgctgctcgacatgtgggaacacgccttctacctgcagtacaagaacgtcaaagtcgactttgccaaggcgttttggaacgtcgtgaactgggccgatgtgcagtcacggtatgcggccgcgacctcgcagaccaaggggttgatattcggctga

SEQ ID NO: 67 - DNA, 303 bp, Mycobacterium tuberculosis
atggcagagatgaagaccgatgccgctaccctcgcgcaggaggcaggtaatttcgagcggatctccggcgacctgaaaacccagatcgaccaggtggagtcgacggcaggttcgttgcagggccagtggcgcggcgcggcggggacggccgcccaggccgcggtggtgcgcttccaagaagcagccaataagcagaagcaggaactcgacgagatctcgacgaatattcgtcaggccggcgtccaatactcgagggccgacgaggagcagcagcaggcgctgtcctcgcaaatgggcttctga

SEQ ID NO: 68 - DNA, 288 bp, Mycobacterium tuberculosis
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag

SEQ ID NO: 69 - DNA, 1383 bp, Mycobacterium tuberculosis
atgacgcagtcgcagaccgtgacggtggatcagcaagagattttgaacagggccaacgaggtggaggccccgatggcggacccaccgactgatgtccccatcacaccgtgcgaactcacggcggctaaaaacgccgcccaacagctggtattgtccgccgacaacatgcgggaatacctggcggccggtgccaaagagcggcagcgtctggcgacctcgctgcgcaacgcggccaaggcgtatggcgaggttgatgaggaggctgcgaccgcgctggacaacgacggcgaaggaactgtgcaggcagaatcggccggggccgtcggaggggacagttcggccgaactaaccgatacgccgagggtggccacggccggtgaacccaacttcatggatctcaaagaagcggcaaggaagctcgaaacgggcgaccaaggcgcatcgctcgcgcactttgcggatgggtggaacactttcaacctgacgctgcaaggcgacgtcaagcggttccgggggtttgacaactgggaaggcgatgcggctaccgcttgcgaggcttcgctcgatcaacaacggcaatggatactccacatggccaaattgagcgctgcgatggccaagcaggctcaatatgtcgcgcagctgcacgtgtgggctaggcgggaacatccgacttatgaagacatagtcgggctcgaacggctttacgcggaaaacccttcggcccgcgaccaaattctcccggtgtacgcggagtatcagcagaggtcggagaaggtgctgaccgaatacaacaacaaggcagccctggaaccggtaaacccgccgaagcctccccccgccatcaagatcgacccgcccccgcctccgcaagagcagggattgatccctggcttcctgatgccgccgtctgacggctccggtgtgactcccggtaccgggatgccagccgcaccgatggttccgcctaccggatcgccgggtggtggcctcccggctgacacggcggcgcagctgacgtcggctgggcgggaagccgcagcgctgtcgggcgacgtggcggtcaaagcggcatcgctcggtggcggtggaggcggcggggtgccgtcggcgccgttgggatccgcgatcgggggcgccgaatcggtgcggcccgctggcgctggtgacattgccggcttaggccagggaagggccggcggcggcgccgcgctgggcggcggtggcatgggaatgccgatgggtgccgcgcatcagggacaagggggcgccaagtccaagggttctcagcaggaagacgaggcgctctacaccgaggatcgggcatggaccgaggccgtcattggtaaccgtcggcgccaggacagtaaggagtcgaagtga

SEQ ID NO: 70 - DNA, 4344 bp, Plasmodium falciparum
atggcgagacgtatgaacttattaactattgaaaagaacattcagcatttatggagggaacataatgtttatgagaaagattttagcgatatgaatgaaagccgatatacaggcaatttcccttacccttatatgaacggtttattacatattggtcatgcttttactttaagtaaattggattttatagttcgatataaaaatatggtttgtgataatgtgttacttccgttttcgtttcattgtacaggtacacctattgttgtgtgtgccgataaattaaagaatgaattaaataagaagaatatacaagattttgaagatatatcatatgagaagaaggaagatgattattctttatgtagatctattagtgatgataaaaataatgtagataagaataaattagttgaagatacggattcaaataaaaagaatacagatgtgacaatatttcgttcgaataaaagtaaagctcaatcgaagggttcaaaacaaaatactcaatatgatattatgaaacaaatgaatataaaagatgaggaaattcatcttttccaaaatccagaatattggtgttattatttttcatcaaaggctaaggatcatttatattcatttggattatattgtgattggagaagatcatttataacaacaaatattaatccatattatgataaatttgtaagttggcacattaatactttatataaaaaaaatcttatatattatggtagtagagttactatttttagtagatataataatcaagcatgtgcggatcatgaaagatcagaaggtgaaggagtcaaatgtcaagaatatacacttatcaaaatttttgtttcgaatgtcaaagatttttattctatatttatgaacagcataagaagtagccaatcggtacttaacgcatgtatcgaaaaaaaagaagcacccaacaataataataacaaaataaacaataacaaaatcaacaataacaaaatcaacaataacaaaatcaacaataacaaaagtaataataacaaaagcaataataataataattgtggtagtagtgccaatataagtaataccttttttacagatttcgaaaaaggagaagaagatttaaaaaataaaatatggaatgaagacttttttgtaaaagataaaaaagttatcttccttggtagcaccttaaagccagaaactgcatatggacaaaattatactttcatcaaccctaatgaatattactatttaaccttaggatttgataaacaaaatttacattatggagataaaagttatgtaaataatattatgacgaaagaagaaattattaatagctgtccaaacatttatgtgtgttcagaaaatagtttatataatttagcatatcaaggaataatacctcttttaaaaaataaaaatataaaaaatgtacaaattccaaaaagtgaagataatactaatgatgatgatactttagaaaaaaaaaacaatgatgtaataacaaaaaatacaaacaataataataatgaaaataataataatatgaataatgtgtataacttagacgatgttttcatattaaataaaataaagggtgaacattttgtaggactagagacatatacaaatatatcgaagataaaaaatttatatatcttacctatgactacaataaaaatgaatatctccacaggtattgtaccctgtgtgtctagtgatagtacggatgattatgcttgtttagaagatatacgaaaaaaaaaaaattattattgtgaaaagtataatttaaaagaagaacaattaaaaaataatagtgaatcatgtatagaactaccagaaataggtaataatactggtaaatattattacgaaaaggaaaaagtatcatcttataaagatgtaaaattacaaaaaataaaagaggttttatataagaaacaatattttgaaggaataatgactgtcgatccatataaaggtatgaaaacatttaattgtagaaaattagcaaaacaaaatattattagaaatttggatgggttcttatatagtgaacctgaagttatggtaatagatcgaaataacgtcaaatgtattgcagctttatgtaatcaatggtatataaattatggaaatatggaatttaaaaaagatgttctaatacaattaaaaaagaataatttccaaacatataatgatgtattatataaacaattgcaacatgtcattttttggttagatgattggtcatgtagtagatcctatggattaggaacttatatgccacaatttgatcaaaataatcaaacgaataaaaatgttgataattatcaaaataataatgagtatgtaataccatccaatgatgataataaccaaataaacaatcaccatatcaatgttgtgaaggaggaagaagaaaagaatgtacatataaaaaacaattcacgaaaagaattaatagagagtctatccgattcaactatatatatggcgtattatacggtgagtcattttttacaaggaagtgttgatggtcagaaaagaggtttattagatattagcgcagatgatttgaatgatgctttttttgattatatttttgatattagtgatgatacaagtaatatatctaaacatatatctaaggagaaattagtaagaatgaggagagaatttacctactggtatccttttgatgtacgtatatctggtaaagatttaatttttaatcatttaacaatggctttatttaatcatgttgctatatgggggaaaaaagaaaaatatgatcgaaataaggaagatgtggaagaaagaagtattttagatagacaaacagaaatattaaatgagttagaaaatattgatttatcaagttatgagaaaataaaatatttcccacgttcttttttttgtaatggacatgttttagtgaataaagaaaaaatgtctaagagtaaaggtaattttataaccttagaagaaagtatagctttatatactagtgatgggaccagaattgccttagcagatgcaggagattctatagaagattctaattttaatactgatacggcaaatagtgctattatgaagttatataatttgataaatttttctatagaaacaaagaataatgtatatatatttagatgtggtgagaaaacatttaatgatttgatatttgaaaatgaaataaattatttaacaaataaatgtaaagaatcctatgaaaaattattatttcgagatgttttgaaatatggtttttatgatatgttattaaaaagagatacctatagaatgatgtgtgataaaatacatatgcataaagaaacagtaaattttttcatagaaagaatctgtataattattaatcctattattccacatgtaaccgaacatatatggacttatattcttaagaaagatacatttttaattaaacaaaaatggccatcacctgaagaaaccaattattctatagttatgcataagcaaaataataatctattgaatgttgtcgaaatatttagaaaatcatatgataaggtaatcaataaatgtaacaagcaaaaggttgtgaaagataaaaatgtttcaaaggaaaacaagaatcacaaagatcttataaacaagataaataatgatgaacagaataaaaagaataatgatacaaatgtagagacacaaaatgattcttctgttcatattgaaaaagaaaacaataatgataatctgaagaagaacatacaacaaataaaacaaataaaccacattagcaacaataataataataaagataatatttgtgatgggcataataacgatatagatgaagatgatgatgaagaaagagatgatgaagaaggtgcaaattattataaagaagatgaagaagaaaagatgaagtttaaagcaatcgtttatgtagcaagggaatataatgatactcaaaagaaaattatagaaatattaaataatattattaataatagtgaagataaaaaattaccaacgaattatataaatctattagtccaaaatacatatgttaataatttaccaaaaaatgaaaaaaaagatatactcagttttgcaacttttctagtaaaagataatgttactttaaataataatcaatatgaattatcattaccctatgatgaaatacaactaattaaggataatgtagatttcataaaaagaagtcttaatttgggagatatacaagtattagaaaataataaaaaaagtgaaattgataatactgatatatataaaatgtctaatccgggatatccatccatatatatatacaacactgaaggataa

SEQ ID NO: 71 - DNA, 6933 bp, Plasmodium falciparum
atgaaagaatgtaacaaaagaaaaaacttgaaaacatatgaatataataattataaaaatatagatgaaacatataaaaaaaatgatcatatttataaaatattattaaaggaaggaataaattatattgaaaatgaaaataatgaaataattaaaaacgatatgtatatatatactttattttttaatcctataaaggaggataagaacaaatctatagtctataatgaagagaaatgtgtaaaggaaaaagaaaaaaggaaagaaaaaaaattatacaatacactattatatttatcaaatataaaacataaaagaaattcttttaatttccctatatatacatcgtacaagtataattataagaaaaccaaatatctatttttaaaacataaaatttttaagaaaagaaaaaataaaaataataaaataaaaccagaaaaaagacaaataaataatatcttataccatatacatcctcttaaccaaatgaacaaaacaaatgtcgacttaaatcgttttacaaataaatctgtagaagataattattgcacaaagttgttttttaataattatgataaggataattatataatttataataaaaatgtttgtaaaaaatttattcacttgcaaagtgtatcagcggcattgtatagttcggcatgtacagcatctgttgatcatataaatggtaatataaatgatcacccaaaagaacttataaaagataatatatgtaatcactcctctgaagatgatatatctacatataataaagtactcctttataaaagagtaaatataaaaaatgataaaaaaaaaagtagtcaaacaactaatacagaaggaagaaaaatatcacctttggagatcaataaaaacgatacccttagtaattcaagagataataaaaaagtgtgtcatttaaaaaataatattttttgtgagaatgaagacaacatttttcatgaagcaaaaagtaaggaatgttctatacttagctccagtcatcaaaatggaaaagataaaaaaaaaaaaataaaaaataataataaaataataataattaataaaaatggtacatgtgatatgaatataactagtagtaaggataatagaccacctagtgagggaataaaaaattatgataaatattatacgtcaaatgatatgttatcttatcataataacaatgtgcctattagtagttatatatatcacgataaacctaagcaaaatgaaaaaataaatgtggatcatagtattacaaaggttgtaaaagaagaaaaaataaatcttcagaatgatataaatagaaataattatcattataatgataattattataatgtattaaagtatgaaaaaagaaaccgaaaaaatcaaagacatcgaaagaatgatataatacgaaattataaaaatgcttatacccaaactgattttatagataaagacaaaagtggaaatatatcaaatgaaaaaaatataatgataaagaataaaattaacataaataacaatggagaattaaaaaatattcaaaataatccatatatgaaatatatctacaataataatcatgtattccagaaaaaaacagaaaaatgtatctcttcaaatgatgataaacattcatttagtaaacaaaatgaatcaaatgtttctaaggaggaatatgattttataaatactacatctatgtatgataaatatcaagagcaatttagtaaactcgttaagacacctcacaacaaaaagaagcatacaaaggttggtatgaaaaataaaaataatatctataataataacaataaaatgtataatcataaaaataatatctacaataataacaataaaatgtataataataacaataaaatgtataaccataacaataatgtgaataatcataacaataatgtgaataatcataatagttattattataattattataacaatgtttatggatttggtgagacactaaaatttatgaagtatagagaactactaaggaaatatatgggtacattttataattatagttccttaaaatgtgctagctatattattatttactcattaatagtaaagattattacacagatatcgaaaaatataacaacttctaatttagctaatgacacaaatgatatgaacggtattaatggaaataaattaaatacaaatttgtttttaaatatatatgatgctgatggtattattcaaagtatatgttatattgttttattaatatgtattatattattaataaaattagggataccatcaacatatttatttgctggtaggttaactatattatttatgcttttatttgttatacaaatacctataatgatatatgcaaattttcttttttcaaaacaggaatcaacacatattaataataataatgataataataatgataacaataatgataataataatgataataataataacaataatgataattattataattattataattattataaaatgggtgtgaaatatatttatgatagaaaaaaagattattttaatttacaattacaaaaattatggtccctattttatatagatactatgaatttattaatatatacaatacttataacaatagtgtccttatatgctacttatcataaattaatatatccacatttattaaaattttatctagaccatttttttttaagttcatattcaattgtaaaaaaagaaataattcattttagagtacctaataatattgatgcatattacctgaacaaaatgaatagctacttatttaataccttgtataaaaaacaacaacatggttataaagaaaatgtagaaagtacaactagaagagcaataggttataatatagttattaaaaaggagaaatgttctttttttcttaacctctttacaaaacatgtatcaaataaaattttcccaatgaggaatgataaacatttgaatttctatattatcttctacataggtgaattagataaaaaaggaagaccacatggttttggttattggagaggtataaatttggagggagaagttttaattggatactggtatcatggaatacctgttggtccattcaaatgtagagattttaaaacgggttctggatttatgtgtataaaaattggatatggaaaaacgaactgtgagctcaacgacttggagataggtctggcagacaccgaatgctgtgttagcggggccttttacagaaccttcccaagggtaatcttttacaacttaaatcttacgaataatacctctaaaaataggaaaaacgaaagtaaccatggtatttttgaaaaacgagaatgttgcgaatatttaatcatcaacaaagcgagcaatgatatacttcgtattgattataacaaacttttaagtgacgaagaggaagagaaacaagaagaaaaggagaattcacagaatgacactattatacataatgataaatatgatagtaataataataaaaataatgataaatatgataataatcataatatacataatatacataataataataataataataataatctttatgaacaccttgcggttaatcatattaaggttacacaaaatgaagataaaaaccatgataacaaggaaatcattttaaacatcccttgtaattataaaggagatgaaatacccccctcgtcatttttaaatgatgatatattattagaaaaagacattcttaattacgacgatatctgtgatttaggttatagcatacaaaatgaaatacattcaacatttaataagaatatagaaacaaacaattcaaatataaatattcatgacaacaaaagttggtttagagatgatatttcatataataattcctttgataaagataatagtaatttcttatgtgatatgaatattcctataaaggaagacaaggaaaatatatctatttgtttgaatcatgacacaaataattataataaaaaggaggacatcggtgaaatcaatttaacaaaaataactacaaataaaatgttcagtgatcaaaacaaaatttgcgttctgaaaaatgattctttaccatctctatctaatattaaaaaaaaaaaaattgaaaaaaaaaaaaatattcttaatgtatcaaaaaatggaagttttaagaacacgaacaaattaaatgataataaaaaagatatttgcttcaacccaatattgaaaaataaaaaggaattaataaaaatagaacaagtaaaagaacacggattattatataaacaaagcagttttaatccgcagcaaagtgaaacatgtcataaaaatattaaaggaaaaaatccaaaaaaaaaaaaagcaaaaatgtatgttcataaagaaggattattttcaaaacctaaggtgataaagtattcaaaagaaaaaactacaggacagatgaatgtagatgatgacgaggaggaccgtaaggataaacatgggaattcgtccaaaaaaaagttatacaatattattaagagtaaaatatctagtaagaataaaaagaaggaaatattaaacactgggaagaaaaaggaaaatgcacatttaaatgaatctgatgtatcaaaaaaaaaggatgttgataataatatgaatttattgaggagttcttatgaggaagatcagtatagaagaagaagaataaaaaaaaaaaagaaaaaaaaaaaaaaaaaaggtataaacaataaaaggaatgaaatggaacaaaacaaagatgacaataattatgaaaaagaagaggatgaaggatcttttcgtatatataacaagttgggaaatgaatatatacatgataacaataatgataatcatcaattaacggatgataaacggaaaataaagaaacatataggatatagtatgacgaacaatattttagattttaaaagatttagaggttttaaaagaagaaagaaaaaaacgaaaaagaaaaaaaaaagccatgataatataaacatgaaaagttatcataatagcgatgataataataatgacaatagtgatgataataataatgacaatagtgatgacaataatgatggtagtaatcataataagaataagattaagaataagtataataaatattttcattttaataaaatatacaaaataagaaataatagacgaaggagggcatttttaccaaaagctttatcaggaacattaccaaaattaaaaaggaaaaaaaaaggtagaagattattgttttctgatacgataacaaatgtatatcaaactaagtgtatgaatgtaatattacaaaatttatgtcctcacatgccaaactttggtataatatataacacagatttacatatatcaatagatgctgaaagaggattatatatcactggttatattaataaaaaaaattataactttttagcaagtagaaatacaaaagacaattttgaagataatgaagtaaaaattaaaattataaaaaaacgaaaaaatacatacaatacatttaatcataataaatataatgataaacatatatactcatggatagatgatattaaagaattaagtttagatgggtgggtaaaatgtgaattagctggatgcttagaagcagtcatttttatacatggatataatacaagtcatttagaagctctacaaattttaggtcaaatggcttcttttggtaattttcctaattacataaaactctttttatttaattggccttctggaaaaaatcttctggaattttttatagcaaaggacaactcccaaaataagaaggtgcaccatgccttcaaatcgtttctggatacactacgaaataatggcataagacaaatacatattatcacacacagcatgggaacaaggatgttcctactagcctttcatgatatcgtaagtagcgaattattttcgacgattgaagaaaaggaaaaagatgaaaagtatcaaaacaaaatgaagctaataaccctaactatgatgaaccctgagtattatttaagcgatttcgtaaataaagaatatatatttcttcgctcattttgtacagtgatttcaatatattgtgattctaatgataaagctttaaaatgggcagaaatttttagtggtactaaatccttaggaaaaaatgtgtttgatttaaatataagtaaagaagatatttctaaaaaatataatggacgaggtgtaaattatttatttgatagtaaccaattagattattattctatttctatggaaaataacaaaaaaattcctaataaaaagaatatgaacatacaaaagaatacagatgataaaatagattacaacatagatcataatattatatcatcattttttgattgcgaaaatttagaaattacaaataacaaaaaaaaaaataaaaaattcaaattcaaaatcaaaatagcagaaaagttaagaacccttgttcactttttttttggaaaagaatatgtaaaaaacaaaacttatattaatgatcaaaattcattacaaataaatgacaatgacaatgatgatgatgtagatgaagatgactgttctttaaataaaaataaaaaacaaaatattttacatcagccacctatgttcagaaatacattactagtatttactggaatagaacctacttattgttataaagataatcgtgattggctagatgtagatgtcattgatactacttggttaggttcaaatgtacatacattgagacattcttattggtcattaaatagagaaattattgaagatataagagaattgatagttactagaaaaagagcaagacaaagaacatctcgattggatagaagagaagggaatgtatgggtatatcgtgtagcaccttctcatttaaaatcaatatttgattcggacatataa

SEQ ID NO: 72 - DNA, 3642 bp, Plasmodium falciparum
atgaacgctgagaatttgaaaggtaaagatgaaattatatttgaggagaaggaaaagaatgtaaggaataatatgaaggatcacgataataacaatttatatagtgaagatattatactgaacgaaaaaaaggataagaatcataatgatgattatgatggaaactatgttgggatgaaaagagataaagaaaattttgtaagttttaaagatgataaaaataatttaataagtgtaaaaccatcgacattaaaaaatagtttgatatttcaagataaaaagaataatttaaaaaagaaagttaccattaaaggtgattatatacgtaaaaagaatgatgaaatatatcttgataagaataatttgtttaatgaaataaatttattgattaaaaagaagttaaaaattaatgatgttataaaactcgatttatttcataaagataattctatacaaataaaaccatatgttatatttgatgattataaaaatgtaaatatagagaataaaataagtatagaaataataaattttttaaaaaactgtttagaaaataataaaaaatatattaacattcatagtagtgttgttgaattaagaggtacatttaatgatgaactaattaatctacctatacttaatgaaactatattacaaaagattagtaataataattataaatataaatttaatatagataatgaggtgcttaattatatatatgttgatttatttaatatacataagagcaaatataaatttaaaatattagaaaaaatttttgaaatcgaagaattatattattatgatgtcaaacaagagaaagaaaaaaaaaataaaataaatatgaacaaatcagaagattataaaaatgatgaacaaggaaaagaatttctaaattcgtctattaatgaaaataataatgaaaacagtaaaaatgatgacagctcttctagtgtatgtgcggatagtgatgtgttaataaatttatataaagaagttaaaaaggaagaggataaggaaagaaaaaaaaaggatgatgaaattatgaatgaacatgtacatgagaaattaataaaaaagaaaaaaaaaaaaaaaaaaatcgataaagataaattatatattgaaaaattaaaaaagttagtagaagaaggtagtgcagatgaatcctataattcatctaatgataagagtagtgaagattacgatgatagaggcaatgataaaaatcaaatattatataaaaggaaaattatatattccaaatggaaagctccagtacaaataaaaaaaaaggaaaaaaaaaaaatattaaagaaaaaaaataaaaatgaaattgataagaaggaggatgatcattatgaccaggaatataacaataataatgataacaataatgatgataatagtttgacaaatataaaaatatttaaagaagtagaaaatgatattgtgtgtgtttattatcctaatagctcatacaaccaatgcttatgtttatcttcttatgaatatataacaaatggacaagaacatattttaccaaataatcatgatatttatggccataataataaaaatgatacatatatgaacaatgcaagtttttgtgtaaatactcttaacgaagaaaatattaagataaagaaaagatatgtaaaagatatatccatatataacttttgttgtgatgacaagtataagggatgtaatttatatcttcatattgctaaaacgaaaaaactgaaaaattgtaaaatgaattataatattccgtatgagcactttttagaaaattatgatataataaaaatgtacgaaattaaaaaggacaaagggaatgatgataaaaggaatgataatgtgaagaatgatgatgaaatgaataaccatgtgacgaatgatgatgaaatgaataaccatgtgacgaatgatgatgaaatgaataatcatgtgaagaatgatgatgaaatgaataatcatgtgaagaatgatgatgaaatgaataaccatgtgacgaatgatgatgaaatgaataatcatgtgaagaatgatgatgaaatgaataatcatgtgaagaatgatgatgaaatgaataatcatgtgaagaatgatgatgaaatgaataatcatgtaacgaatgatgatgaaatgaataatcatgcgaagaatgatgatgaacaaattaactgtcgagaaaaaatgaatagtgataaccaaataaataagggagagaagaaaagaaacctcatagggaaactgtttgaaatagaaaaagactggaaaataaatatcgatatgcatatagatatagagaaattaataaaggaatataaaaagtgtaataatagaaacgaggaaatacacattagtgataataatcttacggatgataaaaatgaacaaaaatataagtctaataaattacattatatgatatataacttttataaaaatttcgattgtttaatgaatttaataaatagattaaaaaacaattcgattaaatatgaatattatttccctaaaatgattggtgatgttaatgaagagatacgaaaacattatgataagaaaaaagttatattattaaaaaagagtaatattaaatatattagggtatttaataatgaagtcaaaaggattatgattctattttttgtatcttataattcgaagatcttagatttagcttgtggacatggacaagatatgttaaaatataatagtgtacaaaataaagtgtatgtaggtattgatttatcaaaaaaggaaatcgaattagcaaaagaaagattaaatcaaaatgatatgaaaggcttatgtaataatgacaattttatttttttacaaggtgatattttaaataataaattttataggaaatggaaaagtaaaaatataatgttcgatattatttctattaatttggctttacattatgttatatataatgaaaagtcgtcaaaaaagtttttcaaaattattgaaaattttttagaaaatgatggattgcttttagcaaccactatatctactgtaacgttgactgactttctaatgaagcgatcgaatattgaaatgaatggagataatattacgattacccttgagaacgacctgttcactataaaatttgaccaagagaacttgttgaaaatatttaaaaacaaaatatgtcttgaggaatttatagaatttattaataataattcaggttctcaaataaaatatgattacttcagtaatttgataagatattctcttgataatgtagttggtataaaatattatttttatttatatgatacaattgatgctcacgaatttgttattcctcagacatatttaaaaaaaaagttagaagaattggatatggttgaattatttaataataccgctataatgtttcttcattatataacaaataatttagaaacgtatgaaaaatatgataatattaaatatttccatttattaaataaaactattgatcataatatttttaaaaacattaaagagagaattaataaaatacatggctatagtagggactcacaaatatattatgatatatgttctttatatcatgtatatgtatataaaaagaattttgatgcaactatattcggaactatgtaa

SEQ ID NO: 73 - DNA, 2238 bp, Plasmodium falciparum
atgtcaacggaaacattcgcatttaacgccgacatcaggcagttgatgagtttgattatcaacactttttacagtaacaaagaaatatttttaagagaattgattagtaatgctagtgatgccttagataaaataagatatgaatcaattacagatactcaaaaattatctgctgagcctgaattttttattcgtatcattcctgacaaaaccaacaatacattaactattgaagattcaggtattggtatgacaaaaaatgatttaattaataaccttggtactattgcaagatcaggaaccaaagcttttatggaagccatacaagccagtggagatatatctatgattggtcaatttggtgttggtttttattcagcctatttagttgctgatcatgttgttgttatctccaaaaataatgatgatgaacaatatgtttgggaatctgctgcaggaggttccttcacagttactaaggatgaaaccaatgaaaaacttggaagaggtacgaaaattattcttcatttaaaagaagatcaattagaatatcttgaagaaaaacgtatcaaagatttagttaagaaacactctgaatttatctctttcccaatcaagttatactgtgaaaggcaaaatgaaaaagaaatcaccgcatctgaagaagaagaaggagaaggagaaggagaaagagaaggagaagaagaagaagaaaaaaaaaaaaaaacaggcgaagataaaaatgctgatgaaagtaaagaagaaaatgaagatgaagaaaaaaaagaagataacgaagaagatgataacaaaactgatcatccaaaagttgaagatgttaccgaagaattagaaaatgctgaaaagaaaaaaaaagaaaaaagaaaaaaaaaaatacacacagttgaacatgaatgggaagaattaaataaacaaaaaccattatggatgagaaaaccagaagaagttacaaatgaagaatatgcaagcttctataaatcattaacaaatgattgggaagaccatttagctgttaaacatttctctgttgaaggacaattagaatttaaagccttattatttataccaaaaagagcaccttttgatatgttcgaaaatagaaaaaaaagaaataatatcaaattatatgtaagaagagtttttattatggatgattgtgaagaaattattccagaatggttaaattttgttaagggtgttgtcgattcagaagatttaccacttaatatttcaagagaatcattacaacaaaataaaatacttaaggttatcaaaaaaaaccttatcaaaaaatgtttagacatgttctcagaattagctgaaaataaggaaaactacaaaaagttttatgaacaattcagcaaaaacttaaagttgggtatccacgaggataacgcaaatcgtacaaagatcaccgaattactccgattccaaacctcaaaatcaggagacgaaatgatcggattaaaagaatacgtagacagaatgaaggaaaaccaaaaggatatttactatatcaccggtgaatccatcaatgctgtttctaattctccattcttagaagctttgaccaaaaaaggattcgaagttatttatatggttgatcctattgatgaatatgcagtacaacaattaaaagattttgatggtaagaaattgaaatgttgtaccaaagaaggtttagatattgatgattcagaagaagccaaaaaagatttcgaaaccttgaaagctgaatatgaaggattatgcaaagttattaaagacgtattacacgagaaagttgaaaaagttgttgtaggacaaagaattacagattctccatgtgtattagtcacatcagaatttggatggtccgcaaacatggaaagaattatgaaagctcaagcattaagagataattccatgactagctatatgttatccaaaaaaattatggaaatcaatgctcgtcacccaattatatcagcattaaaacaaaaagctgatgcagataaatcagataaaaccgttaaagatttaatctggttattatttgatacctctttattaacatctggttttgctcttgaagaaccaactaccttttctaaaagaatccacagaatgattaaattaggtttatcaatagatgaagaagaaaacaatgatatcgatttaccacctcttgaagaaactgtagatgcaaccgattctaaaatggaagaagttgactaa

SEQ ID NO: 74 - DNA, 3411 bp, Plasmodium falciparum
atgaacagttcatctagctacacatttatcagttggaaggtcgtcatggaattgatcataaaaaaaccatatatagaaatatttgaaaagatcttaaaactgtttacccatatttgtgaaaatgttcattttaaattaacaagaaataagctagaattaagcggctcaaataatttaacaaatgaactggtgatacatattgataagaaattttttatattaaatgatgtaaatggagataagaagaatataattaatggcactgtaaaatcaaaagatttttacaattgtatatataatcataagatagttaaacatctgaggagtacttattcacaacatggatcttataatagtcacaagaaaaatgattcaccttttctaatgaaagatgatatgaatgaagaaggaagaataaataatacaaataatgatgaaattaaatcaatcgtttgtgatggtatgtatccagatgataacaataagtcatattttaggaaggataaaaagtggaatatacatctttctaagataactctgaaatttaataatataaatcataatataaataataatatggatggtgatgatcatgatgacaataataataataataaattagaaataattataaaatttaaaaagtataacacctattttagcgcaatattaaaattgaaaacgttcaacacaccaatgaaaaattatatttataaaaatgaatctattatacaaatagatccaaccctttttttacttaatcttaaagatctatctaatgaaaagaatatttttttaaagaatacagataattcatttatcatcagttctttagaaacttgtgatttcagtttaaataaagaaagaataaaaagagaacaatttttttataataataaaaatatatgtatcccatctagtaaaacgaaatatttttttaaaaataaaaaattccaggatcataatatgtctcttcctttaaatgaactaaagaccataattaaattctgttcagacttaaatttattatgtttgttttctacaaaaaactttaaagaaaatcttattatatattttggtaatataatatcttatatattagaaaaaaacaaaaataaaataaaaaaaaaaaataaaaataaaacttttcaacacaagcagcatacatatatgctgaacacaaaccatgtgaaatatttaccaaatatatcacgtgattataaatatgattacacgtttgattcgtctaaaaaatcacacgtaagtagaaataaaatccatgatgatgatgataaatatataaattcaaatttgtatatattatcaaaaggggaattaaataataatgacagccacatttatgataataaaaacaattacgataataataacaaatcactagttttttatttatctgattatacatcaagcgatgaatatgattctagtatggatgaattcgatgatcaaatatatttaccagtatacgataatgaatatacaaaggatgacaccaattttaattataatcatattataacaggatgtatacattttacttcatattttaatatatcatgtgattttaatgaatatgaaaatgataagaaatatgaacacgaagatgtttttcaacaacctatggatgcatttgttataaataataatgatgataatgatgataataatgataataataatgataataataatgataataataataataattattttgatgatagtaaaaaagttaagaagatacaacataaggaaaaaaaattatctatagaaaaaaaaaaagaagatataatttatgataatacaaaaagttgtagttttaattatgaaaggaataatttaaaaaatataagagaagaaggaaaagaagaagaagaaaaaaaaaaaaaaaaaaattcgtctatctctgaagatatagataaggataacaattatcatgttatgcaaaatattaaaaataaagatattaagtatgaccgaaaggaagacggtttcagtgatacttatggtatgtttaatcatttgaaggaaaacaaaagttatgaggagaatagaagaaaaaaacatcatcttgttttgataaatgaaaaatgtgaagagtctgaatatgatcaaaatggtgagtgttcacatgtcccaaaagatgattattcatatgatccaaataatgattgttcatatggtccaaataatgagtatccatatgatcaaaacaatgaatgtccatataatcaaaacaatgaatgtccatatgaccaaaacaataatattaatacaactttcaataacctcgtacaagaacaggaaaaagaaattatttgtaattccaacatttatgaagaattcaattatgatacctatatgaggcaaaatgaagagacacaaaatgtgtataaaaatatgcacgaaaaaataaaaaaacttgatataaccttggaaaatttaaagcaaataaataaaaagaagaaaaaaatactggattatcaaaaggatgatgtaaaaaaaccctttttggttttttccttaccaaaaagtagaaacatcaaaaaaagaaggaaacccatcgaatatatttctgaagtgaataaaagaaataaaagaaataaaagaaataaaagatcatccaaggatttgttatataatgatgtggaaaataaaaggtatgataacaattcggtgtcgtgtagaaatgaggaaaatgaaattacaagtgacaatttttattccttaaatgatgacaatgaagaggatgaaaataatgattattatgatgaaaataataataattatgatgatgaaaataataattatgatgatgataataataattattatgatgatgataataataactattatgatggtgataataataactattatgatggtgataataataactattatgataattttccggatgagacttattacaacattcgtcataataaacatggtgtgggagaaaatatggaatatatgaactacttcaataatatatacgacaaatataacatgagtaacaaaacgaataaagaaaaaacacatcacataaaaaataaaatatataaaaatcaattaaactatagaaattattatatgagtgaaaaagataacaatattcagtatataaaagggggaaatgaaaagaatgagaatgatgaaaagttttataataattacaaacatttcaacatgtctagattgtcacataatctacatgataaggcacctctcacaaatcaatattcagggcctaagaagaatacacgattaattatgaaaaattattcttctcatattctaaaagatgaaataaaacatcctagtgttaattatgataatgacatatttaattataaaaaatgtaagttacaaaagaagtcataa

SEQ ID NO: 75 - DNA, 1053 bp, Plasmodium falciparum
atgatcgtttgtaaaaaaatttttaatgagtccactaaattatggaaacctaaatatttaaataataatataaaatggtatcaaagtattaataaggaaaaaggtgttaataaaatatatactcaaaaaaagttaacaaaaagagagaagttattaggaaatataaaaccatttatcaatgataaatatgataatcctgaatgtaatgtgattgcttattatttaaataattttttaaatttgaatatattgaaaagtgcatatgaaaatattggttgtaatgttttatataaagaaccatatttatatgtagaaagttgtgaatattttaaacaacagaatagttcagctgtattttttaaaaatggatgtgttgtgatatggaatatgaataaaaagaatatgaagaattttttatatttttgtaaaagttatataaatataaatgaaaatatagataattatgattttgaagaattagaagtacaaaatgttaataataaaagttatgtatcaaattctattatttatttaacaatgaataattatagaattacagataagatttctttttcatttggattattgagtgctgtgagattaaataatttagaaaaaaaaattgaaaataaattactctatgaaaataataatatcgaaacattaaaaaaaaaaattaaatcaactaatttggatttattatcaaaacaacttttttcttctaaaattactttacataatttaagatatgaattaaatattgaacaagatatacttgatgtaccagaaacattatgggaattagaatatcaaaagaaattatttttaaatattcttaatatctttgatattaaacaaagagttgatttgttaaaccatagattaacttggacatttgattatttaaattccttcctcgattatgttaatcaaaaacattcttcaaggttggaaagaattattatcatgattattggattggagctaattctcggtatcatgcaaatgattaaaaccgtggatttaggagttcacaaaaaaaaggaaagttaa

SEQ ID NO: 76 - DNA, 1758 bp, Plasmodium falciparum
atgaatagaatcttgtctgttacattatgtttgttttttatatatttatatatatataaaacatatggaaaagttaaaaatacagatgaaggattatcaaatatctatggagcaaaatattatttaagaagcggattatttaatgaaaaaaatggtaaaggacaaaaatatgaagatttagaagaagaaaaagaaggcgaaaatgacgacgaagaggattcaaatagtgaagaatccaataatgatgaagaaaatgaactaataaaaggtcaagaaggtgttgaacaagaaactcacggatcagaagatgaagtaagtaatggacgagaagataaagtaagtaatggaggagaagatgaagtaagtaatggaggagaagatgaagtaagtaatggacgagaagataaagtaagtaatggaggagaagatgaagtaagtaatggacgagaagataaagtaagtaatggaggagaagatgaagtaagtaatggacgagaagataaagtaagtaatggaggagaagatgaagtaagtaatggacgagaagataaagtaagtaatggaggagaagatgaagtaagtaatggacgagaagataaagtaagtaatggacgagaagataaagtaagtaatggaggagaagatgaagtaagtaatggacgagaagataaagtaagtaatggacgagaagataaagtaagtaatggaggagaagatgaagtaagtaatggacgagaagataaagtaagtaatggaggagaagatgaagtaagtaatggacgagaagataaagtaagtaatggaggagaagatgaagtaagtaatggacgagaagataaagtaagtaatggacgagaagatgaagtaagtaatggacgagaagataaagtaagtaatggaggagaagatgaagtaagtaatggacgagaagataaagtaagtaatggaggagaagatgaagtaagtaatggacgagaagataaagtaagtaatggacgagaagataaagtaagtaatggaggagaagatgaagtaagtaatggacgagaagataaagtaagtaatggaggagaagatgaagtaagtaatggacgagaagataaagtaagtaatggacgagaagataaagtaagtaatggacgagaagatgaagtaagtaatggacgagaagataaagtaagtaatggaggagaagatgaagtaagtaatggacgagaagataaagtaagtaatggacgagaagataaagtaagtaatggaggagaagatgaagtaagtaatggacgagaagataaagtaagtaatggaggagaagatgaagtaagtaatggacgagaagataaagtaagtaatggacgagaagataaagtaagtaatggacgagaagataaagtaagtaatggaggagaagatgaagtaagtaatggaggagaagatgaagtaagtaatggacgagaagataaagtaagtaatggaggagaagatgaagtaagtaatggacgagaagataaagtaagtaatggaggagaagatgaagtaagtaatggacgagaagataaagtaagtaatggaggagaagatgaagtaagtaatggacgagaagataaagtaagtaatggacgagaagatgaagtaagtaatggacgagaagataaaggaggagctggaacggatggagaactttcacataatagcgaaagtcatactaaaaacaaaaaatcaaagaatagtataattaatatgttaattggaatgtga

SEQ ID NO: 77 - DNA, 4791 bp, Plasmodium falciparum
atgaaacatattttgtacatatcattttactttatccttgttaatttattgatatttcatataaatggaaagataataaagaattctgaaaaagatgaaatcataaaatctaacttgagaagtggttcttcaaattctaggaatcgaataaatgaggaaaagcacgagaagaaacacgttttatctcataattcatatgagaaaactaaaaataatgaaaataataaatttttcgataaggataaagagttaacgatgtctaatgtaaaaaatgtgtcacaaacaaatttcaaaagtcttttaagaaatcttggtgtttcagagaatatattccttaaagaaaataaattaaataaggaagggaaattaattgaacacataataaatgatgatgacgataaaaaaaaatatattaaagggcaagacgaaaacagacaagaagatctagaacaagagagacttgctaaagaaaagttacaggggcaacaaagcgatttagaacaagagagacttgctaaagaaaagttgcaagaacaacaaagcgatttagaacaagagagacttgctaaagaaaagttgcaagaacaacaaagcgatttagaacaagatagacttgctaaagaaaagttacaagagcaacaaagcgatttagaacaagagagacgtgctaaagaaaagttgcaagaacaacaaagcgatttagaacgaacgaaggcatctacagaaacgttgcatgagcagcaaagcgatctagaacaagagagactagctaaagaaaagttacagggacaacaaagcgatttagaacaagagagacttgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagatagacttgctaaagaaaagttgcaagaacaacaaagcgatttagaacaagagagacgtgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacttgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatctagaacaagagagacgtgctaaagaaaagttgcaagaacaacaaagcgatttagaacaagagagacttgctaaagaaaagttacaagagcagcaaagcgatttagaacaagagagacttgctaaagaaaagttgcaagaacaacaaagcgatttagaacaagagagacgtgctaaagaaaagttgcaagaacaacaaagcgatttagaacaagagagacttgctaaagaaaaagacttgctgagacttgctaaagaaaagttgcaagaacaacaaagcgatctagaacaagagagacgtgctaaagaaaagttgcaagaacaacaaagcgatttagaacaagagagacgtgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacttgctaaagaaaagttacaagagcagcaaagcgatttagaacaagagagacgtgctaaagaaaagttgcaagaacaacaaagcgatttagaacaagagagacttgctaaagaaaagttacaagagcagcaaagcgatttagaacaagagagacttgctaaagaaaagttgcaagaacaacaaagcgatttagaacaagagagacttgctaaagaaaagttacaagagcagcaaagcgatttagaacaagagagactagctaaagaaaagttacaggggcaacaaagcgatctagaacaagagagactagctaaagaaaagttgcaagaacaacaaagcgatttagaacaagatagacttgctaaagaaaagttacaagagcaacaaagcgatttagaacaagagagacttgctaaagaaaagttgaaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagactagctaaggaaaagttgcaagaacaacaaagcgatttagaacaagagagacgtgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagatagacttgctaaagaaaagttacaagagcaacaaagcgatttagaacaagagagacgtgctaaagaaaagttgcaagaacaacaaagcgatttagaacgaacgaaggcatctacagaaacgttgcatgagcagcaaagcgatctagaacaagagagacttgctaaagaaaagttacaagagcagcaaagcgatttagaacaagagagacttgctaaagaaaagttacaagagcaacaaagcgatttagaacgaacgaaggcatctacagaaacgttgcgtgagcagcaaagcgatctagaacaagagaaactagctaaagaaaagttacaggggcaacaaagcgatctagaacaagagagactagctaaagaaaagttacaggggcaacaaagcgatctagaacaagagagactagctaaagaaaagttacaggggcaacaaagcgatctagaacaagagagactagctaaagaaaagttacaggggcaacaaagcgatttagaacaagagagacttgctaaagaaaagttgcaagagcgacaaagcgatttagaacaagagagacttgctaaagaaaagttgcaagaacaacaaagcgatttagaacaagagagactagctaaagaaaagttgcaagaacaacaaagcgatttagaacaagacagacttgctaaagaaaagttgcaagaacaacaaagcgatttagaacaagagagactagctaaggaaaagttacaggggcagcatagcgatttagaacgaacgaaggcatctaaagaaacgttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacgtgctaaagaaaagttgcaagagcagcaaagagatttagaacaaaggaaggctgatacgaaaaaaaatttagaaagaaaaaaggaacatggagatgtattagcagaggatttatatggtcgtttagaaataccagctatagaacttccatcagaaaatgaacgtggatattatataccacatcaatcttctttacctcaggacaacagagggaatagtagagattcgaaggaaatatctataatagaaaatacaaatagagaatctattacaacaaatgttgaaggacgaagggatatacataaaggacatcttgaagaaaagaaagatggttcaataaaaccagaacaaaaagaagataaatctgctgacatacaaaatcatacattagagacagtaaatatttctgatgttaatgattttcaaataagtaagtatgaggatgaaataagtgctgaatatgacgattcattaatagatgaagaagaagatgatgaagacttagacgaatttaagcctattgtgcaatatgacaatttccaagatgaagaaaacataggaatttataaagaactagaagatttgatagagaaaaatgaaaatttagatgatttagatgaaggaatagaaaaatcatcagaagaattatctgaagaaaaaataaaaaaaggaaagaaatatgaaaaaacaaaggataataattttaaaccaaatgataaaagtttgtatgatgagcatattaaaaaatataaaaatgataagcaggttaataaggaaaaggaaaaattcataaaatcattgtttcatatatttgacggagacaatgaaattttacagatcgtggatgagttatctgaagatataactaaatattttatgaaactataa

SEQ ID NO: 78 - DNA, 1869 bp, Plasmodium falciparum
atgagaaaattatactgcgtattattattgagcgcctttgagtttacatatatgataaactttggaagaggacagaattattgggaacatccatatcaaaatagtgatgtgtatcgtccaatcaacgaacatagggaacatccaaaagaatacgaatatccattacaccaggaacatacataccaacaagaagattcaggagaagacgaaaatacattacaacacgcatatccaatagaccacgaaggtgccgaacccgcaccacaagaacaaaatttattttcaagcattgaaatagtagaaagaagtaattatatgggtaatccatggacggaatatatggcaaaatatgatattgaagaagttcatggttcaggtataagagtagatttaggagaagatgctgaagtagctggaactcaatatagacttccatcagggaaatgtccagtatttggtaaaggtataattattgagaattcaaatactacttttttaacaccggtagctacgggaaatcaatatttaaaagatggaggttttgcttttcctccaacagaacctcttatgtcaccaatgacattagatgaaatgagacatttttataaagataataaatatgtaaaaaatttagatgaattgactttatgttcaagacatgcaggaaatatgattccagataatgataaaaattcaaattataaatatccagctgtttatgatgacaaagataaaaagtgtcatatattatatattgcagctcaagaaaataatggtcctagatattgtaataaagacgaaagtaaaagaaacagcatgttttgttttagaccagcaaaagatatatcatttcaaaactatacatatttaagtaagaatgtagttgataactgggaaaaagtttgccctagaaagaatttacagaatgcaaaattcggattatgggtcgatggaaattgtgaagatataccacatgtaaatgaatttccagcaattgatctttttgaatgtaataaattagtttttgaattgagtgcttcggatcaacctaaacaatatgaacaacatttaacagattatgaaaaaattaaagaaggtttcaaaaataagaacgctagtatgatcaaaagtgcttttcttcccactggtgcttttaaagcagatagatataaaagtcatggtaagggttataattggggaaattataacacagaaacacaaaaatgtgaaatttttaatgtcaaaccaacatgtttaattaacaattcatcatacattgctactactgctttgtcccatcccatcgaagttgaaaacaattttccatgttcattatataaagatgaaataatgaaagaaatcgaaagagaatcaaaacgaattaaattaaatgataatgatgatgaagggaataaaaaaattatagctccaagaatttttatttcagatgataaagacagtttaaaatgcccatgtgaccctgaaatggtaagtaatagtacatgtcgtttctttgtatgtaaatgtgtagaaagaagggcagaagtaacatcaaataatgaagttgtagttaaagaagaatataaagatgaatatgcagatattcctgaacataaaccaacttatgataaaatgaaaattataattgcatcatcagctgctgtcgctgtattagcaactattttaatggtttatctttataaaagaaaaggaaatgctgaaaaatatgataaaatggatgaaccacaagattatgggaaatcaaattcaagaaatgatgaaatgttagatcctgaggcatctttttggggggaagaaaaaagagcatcacatacaacaccagttctgatggaaaaaccatactattaa

SEQ ID NO: 79 - DNA, 4554 bp, Plasmodium falciparum
atggcagaggaggtaaatagaaatattaaaaggacgtctgaattgattaataataataaagtagataaaagttttttaaaaaatatcaacctacaaattacggatggagaaataaagttacatttaaaaattattgaagccttatatcctaatatgaaattaaaagtcgataaattaataaatggaagttataatatttttaccaaattcttagtacaatcacatatagatgattttaactcctttataaatgtttatataaaaaatattgctgaaaatataccaataatggaattttcacctcagcaaaataattattctcttttaaatatgaataaaaacaattctgaatctgtaaaatttttcgtgagtgatatacaaataaaaaatccaatgataaaaaatgataaaggagagtatagagcagattatccttatttatgtaaactatctgcaagaacatatgaaggagaattattaataaaaataaatagacaatataaagatgaaatagctagtacaaccatatgtgctgggcatattccaattatgattatgtccaatttatgtaatttaagtaatttaaataaaaaagaattagctcaaaaaggagaagatcaaagcttattaggtggtttttttgtagtatctggaagattaaaagtaataagatatgtaattcatccgaaatataatacattattactaaatgctgataataagatacatataaattgcttattaaatgataatactgttgtaattaattttttaacattaactagaaataattcatatgtttatggattcagatttcaaaatgcagtatgttctcttccttttcatttattattaatgattctaagtccaatcaaaaagaaaagttatatttttaataaaattaaactaggagtagaaaatgaaaatgccattaaatatatagaactttttattaattctatatttctaaaagataccttcaatgaaaaagaactttttgaaaaatataatttaagttacttaggtaggattgcctatttaagaagaggtatttttaaatataatttaaattgctatgaaaaaaaagcaaaagatattttaaaatattgtatattaccgcatataaaaaataattctgaaaaatttgaaaccatgtgttttatgtttaaaaaattaatctatagtaatttcaaattaattactcctataaataaggattctttagaaaatcatgctgtaactacatgtagtaatttattagcaaatttgttgaaagatcaagtaatgaattctttatgtcgtttgtacattagatatacaagatccttttttaaatattttgaaactaaatataacacattcaaattaagtgttaagcaaatatatttgagatacaaaatgtttgtgttagaggaaatgcaagaagaccgcttaaaccaaatatctttgagtaaatcatctgaagatatcaatgattctagttatcaagataataacgattataagaacgaatcaaagaaaaaactcaaatccattttcataaatgaaaaagaaaaattatttaggagtgtcgataattatttcaaagaattatataatgataattatatgtttttagaaaattgtaaaagtttttcaaatacttcatcagctattttatttttctttaaaacaggaaatatctcctcagaaaatttacattatcaacaaaaaagtggttgggtaatagcagcagatgaaattaataatttaagattaataaccaattttagagctatacacagaggaaccttttttcaagatgttaaagtattaactcccagaaaattattaggagaatcatggggtttcatatgccctgtacatactcctgacggtaccccttgtggtttattaaatcatttggctcaatattgtcatattcataatttatctagtaatgaagcacataaactcaatataaaattgtatcttaaaaaaataggtataaatgtaaatttagatgatactagtggtatacatacaatatatgatgatgaaaacattccaattattgttgattcaatacccattacatatataggtgaaaaagattttaatagaacagtgtataaattgaaatatgcaaaaaacaataatttgtttaatttaaaatcatattttgaaataaatgcttatttaaatgaaccgttaattatgaacagtataataattaatacatttccaggaagacttatacgacctttattgaatttgaaaacgaaagatattgaatttatttcaccatcctatcaaccatatatatctgtagctataaacaacgaagatgtaaaaaaaaataatttagctagaaaacttttaaaaaaaaaagaacaagttgttaattcgatgaaaatagatagaacccctggaagtataaagcagaaatttttatatcatcaaaatagggaaaacttattatggaaattaaaaaaggaatcaaaagatggctcatataaatatgaaaaacataaaattgatagaaaattattaaaaattttagatcagaaaaaaaataacgattcagatacaaatacagataattatgttagtagttctgaatatgatacagattcggattatgattatggttatgattctaattctgatggtgattcaaaatctaactctaattttgattcaaaatctgataatatatcaggaagtgataataatatgtcaacaactacatatattgatagtgatgaaaatattaatattgatatgtatgaacaaataccacaaaaatttgaatatatggaactaaaagagacatcgtttttatcctttcttgcttctttgactcctttttcagatcataatcaaagtccgcgtaatatttatcagtgccaaatgttgaaacaaactatgggaatacaaagtttaaataatgtatatacattcacaaataaaatatatcgaatgataactcctcagtttccattagttgtaacaagagattatgaattttatggagtagataattttcctagtggtacgaatgccgtagtggctatattagcttatacaggatacgatatggaagatgcattaataataaataaagctagtgcagatagaggtatatttcgaacacatatatataaaactgaatttattgatttacaaaaagttggagaatctagtaattttgtgtttggaaataatataagatatttaaataataataaaaataataataagaatttggcttacgaaaataaaggaaaatttttaaataaagatggtttaccttgtgtacaacaaaaaataacagagggtagtccattatattcatatattaataaaggtaatgaattaactacttatgaatcttttaggaaccatggagaatatttcgtagactttgtaagtataggaaaaaataattcaaatcaagtagcagttgttaaattaagatcaacaagacaaccacaagttggtgataaatttgcatctagacatggacaaaagggggttgtttctcgattatttcctcaagaagatatgccatttgctgaaagtggaatcgttcctgatattattttcaatcctcatggtattccatctcgtatgactataggtatgcttatcgagagtatttgtggaaaggctgcatctttatatggaaaaagaatagatgctactccatttagaaaatatacgcaacaaaaaagttttaacaatccatggattgacaattgtggtatcaaaggtttcttggaaaaaaatgatacaataaaatctgacttggatacatgtcagaaggaaaaagataaaaataaaaatattaataataataataataataaaataaaagaaaataaatataataactcaaatgatagtaatgcatctattgatagtgatgataatactaacaattgtaatgataaacgtgatgaagaagaaaaaaaagctaatataacttatgatgaaaaaattgattatttcgctaaattgttattaaacaaaggatatgattattatggcactgaattgttatatagtggtatatatggggttccattacaggcacatatatttattggtgtaatttattatcaaaggttaaggcacatggcttatgataaagctcaagtcagaagaacaggaccagtgtgtaacttaactcaccaacccttgaaaggaaaaagaaaacatggtggtataagagttggagaaatggaaagagacggtataatatcacatggttgtagttttgtaatcaatgaaagatttttaatgagttctgatggacatgaatgttttgtttgtcctaaatgtggacttattttatcaccaattatgcaatttaatacaactggaaaaataatgaaaggaagatctattggtggaaaaagtaaaatggctgtatgcaaatcttgtgatgtttcttgtaaaattatttttattccgtatgttatacgatatttattaaatgaactcatttgtttaaatgttaccataaggttgaatatgaaatcagtggaaaatgtttttgacatgaagtaa

SEQ ID NO: 80 - DNA, 3273 bp, Plasmodium falciparum
atgaaaccatatagttcatatagttctgctttttcaaagcaatatatgggtacaaaaagtgtaaaggcaaaaaatccaaccatatattcttttgaagaagaaaaacaaaatgaaaatatgagtttgttaaaatcgttatgttctaagcgtttggttcttccaattcttggaatattatatatcattctaaatggaaattttggatataatggaagttcaaattctggtgcacaatttactgacaggtgctcaagaaatttatactgtgaaacattgccaatcaacccatatgctgattctgaaaacccaatagttgtaagtcaggtatttggtttaccttccgaaaaacctacgtttacgttagaaggtactcctgatattgatcatacaaatattttgggttttaatgagaagttgatgactgatgtaaatagatatcgatattctaataactatgaagccattccccatacaagggagttcaatccacttattgtagataaagttcttttcgactataacgaaaaggttgataacttaggaaggagtggaggagacattataaaaaaaatgcaaactttatgggatgaaataatggatattaataaaagaaaatatgattttttaaaaacaaaattacagaaaacttacagtcagtacaaggttcaatatgatatgccaaaagaagtatacgagagcaaatggggacaatgcttaaagcttattaatcaaggaggtgataaccttgaagaaagattgaacacacaatttaaaaactggtacagacagaaatatttaaatcttgaagaatatagaagattgactgtgttgaaccaaatcgcttggaaagctttatccaaccaaattcaatatacatgcagaaaaattatgaatagtaacatttcttcttttaaacatataagtgaattgaaaagtttagaacagagagcggcaaaagatgcacaagaagaaatgaggaaaagagctgaaaaacagaagaagaaaaaaagtaaaagaagaggatggttatgttgtggtgggggagataacgaaacagttgaaccacaacaagaagaaccagtccaagacgttggagaacatcaaataaatgaatatggtgatatattaccatctttaaaagttagtattaataattcagcaattaattattatgatgcagtaaaggatggtaaatatttggacgatgattcatcagatgctctttatacagatgaagatttgttgtttgatttggaaaagcagaaatatatggatatgttagatggatctgaagacgaatctgttgaagacaatgaagaagaacactctggtgaagcaaatgaggaagaactaagtgttgatgaaaatgtagaagaacaaaatgttgatgaaagtggagaacaacaaagtgatgatgaaagtggagaacatcaaagtgttaatgaaattgtagaagaacaaagtgttaatgaaattgtagaagaacaaaccgttgatgaaattgtagaacaagaaaccgttgatgaaaatgtagaagaacaagctgtcgatgaaaatgaagaacaacaaaccgttgatgaaaatgtagaacaacaaactatagatgaaagtcaagtacaagaagaaatatctactattcaagaaaatatagaagaggtagttagtgaagttcaacaagattcagaggtagatagaactcttcatgttcctgatacaagattctatgatatattaggtgttggagttaatgcagatatgaaggaaatctctgaaagttattttaaattagcaaaacaatattatccaccaaaatattcagttaatgaaggaatgttaaaatttaaacaaataagtgaagcatatcaaatattgggagatattgataaaagaaaaatgtataataaatttggatatgatggaataaagggagttaacttcattcacccaaccatatattatatgttagctagtttagaaaaatttgctttttatactggatctcctcaaatagtaacccttatgaaattcttatttgaaaagaaattaacagtaaatgacttagatacaaaatctgaacatttatcaaaaataatgggagtgtatcaaaaagaaagggaaacttacatatctgaaaatttaatatctagattgcaaccatatatagacagtattagaaattgggatgtacaaattaaggatcaaatatatgaattaatgggttctccatttgatatagcaattatagattctataggatggacattacaatatgtttctatgagtcatatgaaaaaccctaaaaaagcaattaagaaacttgaaacaagatccaagaaaaataaagaaactgtagcatatgaaaataataaactaatgaatatattgagagaatatttcggaaataatgaacaaattaattcaatcacttataatatggaatataatacattaaatgaaaataatgagaatggatacagaaaaattttgaacttgaaccataaaaaacagaaaaaattatttgaagaaattattagttatatagtaaatatatctttatccgatatagagaatacagttaaaaattcagctgaaagtatattaacagttgaagggttagatgaaaaaaaattatcaaagagaattgaatcattaagaatgttagcgaatgctataagaaaatatatattaagaggtaagaaaggtaaaaaatataaaaacaaggatgcaaaaagcttatcaggaaacattgcgaatgaaataaatttaattaataaagaacttcaaaatttaaaagaacatacacaagcaaatatacctgagcatatagaagaaaatgtgcaagaaaatatggaagaaaatgtagaagaaaatgtagaagaaaatgtagaagaaaatgttgaagaaaatgtagaagaaaatgtagaagaaaatgttgaagaaaatgtagaagaaaatgtagaagaaaatgttgaagaaaatatagaagaaaatgtagaagaaaatgttgaagaaaatatagaagaaaatatagaagaaaatgcagaagaaaatgttgaagaaaatatagaagaaaatatagaagaaaatatagaagaaaatatagaagaaaatatagaagaaaatgttgaagaaaatgtagaagaaaatatagaagaaaatgtagaagaaaatgtagaagaaaatgcagaagaaaatgcagaagaaaatgcagaagaaaatgcagaagaaaatgatgaaacaccacaggaacacaacgaagaatatgatgaataa

SEQ ID NO: 81 - DNA, 690 bp, Plasmodium falciparum
atgaaggtctctaaattagtcttgtttgcgcacatattttttattataaatatcttatgtcaatatatttgtttaaatgcttctaaagtaaataaaaagggtaaaatagcagaagaaaagaaaagaaaaaatattaaaaacattgataaagcaatagaagaacacaacaaaaggaagaaactaatttattattcattgatagcatctggggcaatagcatcggttgcggcaatattgggattaggatattatggatataaaaaatcgcgagaagatgatttatattataataaatatttggaatatagaaatggagaatacaatataaaatatcaagatggtgctatagcaagtactagtgaattttatatagaacctgaaggaataaataaaataaatttaaataaacccataattgaaaataaaaataatgtagatgtgtcaattaaaagatataataattttgtagatatagcacgacttagtatacaaaaacattttgaacatttatcaaatgatcaaaaagattctcatgtaaataacatggaatatatgcaaaaatttgttcaaggattacaagaaaatagaaatatatctctatccaaatatcaagaaaataaagctgttatggatttaaaatatcatttacaaaaagtttatgctaattatttatctcaagaagagaactaa

SEQ ID NO: 82 - DNA, 1725 bp, Plasmodium falciparum
atgaatcatcttgggaatgttaaatatttagtcattgtgtttttgattttctttgatttgtttctagttaatggtagagatgtgcaaaacaatatagtggatgaaataaaatatcgtgaagaagtatgtaatgatgaggtagatctttaccttctaatggattgttctggaagtatacgtcgtcataattgggtgaaccatgcagtacctctagctatgaaattgatacaacaattaaatcttaatgataatgcaattcacttatatgctagtgttttttcaaacaatgcaagagaaattattagattacatagtgatgcatctaaaaacaaagagaaggctttaattattataaagtcactcttaagtacaaatcttccatatggtaaaacaaacttaactgatgcactgttacaagtaagaaaacatttaaatgaccgaatcaatagagagaatgctaatcaattagttgttatattaacagatggaattccagatagtattcaagattcattaaaagaatcaagaaaattaagtgatcgtggtgttaaaatagctgtttttggtattggacaaggtattaatgtagctttcaacagatttcttgtaggttgtcatccatcagatggtaaatgtaacttgtatgctgattctgcatgggaaaatgtaaaaaatgttatcggaccctttatgaaggctgtttgtgttgaagtagaaaaaacagcaagttgtggtgtttgggacgaatggtctccatgtagtgtaacttgtggtaaaggtaccaggtcaagaaaaagagaaatcttacacgaaggatgtacaagtgaattacaagaacaatgtgaagaagaaagatgtcttccaaaacgggaaccattagatgttccagatgaacccgaagatgatcaacctagaccaagaggagataattttgctgtcgaaaaaccaaacgaaaatataatagataataatccacaagaaccttcaccaaatccagaagaaggaaagggtgaaaatccaaacggatttgatttagatgaaaatccagaaaatccaccaaatccaccaaatccaccaaatccaccaaatccaccaaatccaccaaatccagatattcctgaacaagaaccaaatatacctgaagattcagaaaaagaagtaccttctgatgttccaaaaaatccagaagacgatcgagaagaaaactttgatattccaaagaaacccgaaaataagcacgataatcaaaataatttaccaaatgataaaagtgatagatatattccatattcaccattatctccaaaagttttggataatgaaaggaaacaaagtgacccccaaagtcaagataataatggaaataggcacgtacctaatagtgaagatagagaaacacgtccacatggtagaaataatgaaaatagatcatacaatagaaaacataacaatactccaaaacatcctgaaagggaagaacatgaaaagccagataataataaaaaaaaagcaggatcagataataaatataaaattgcaggtggaatagctggaggattagctttactcgcatgtgctggacttgcttataaattcgtagtaccaggagcagcaacaccctatgccggagaacctgcaccttttgatgaaacattaggtgaagaagataaagatttggacgaacctgaacaattcagattacctgaagaaaacgagtggaattaa

SEQ ID NO: 83 - DNA, 1566 bp, Plasmodium falciparum
atgtttgaggagaatgagaaggaacaattattgtactatgagaggaataaagaaagtgaaaaatgtcagaaggtttgggaaagtataatttgtaaaggcttaggttatagtaaagaagaaatacgaagagatgtggagagatcgagaagagaaaaggaaagacttttatcaatattatatggtagcgtatttacaggtcctaaatatttgttatattccaagaaatggagaggtaaaacgttgaacgaaataattaatgaagacagagaaaagaacttaaataaaaacaattttaaaaatatggatcttgatagtacatatttttgtgaattatcttttggatacatggacacagcagaagaaaatgatgataaaaataagaaagaaataacaaagcagatggaaaagaagaatgataaagttcctaatataagaaaaggaggaaatacaaataattcacaaaataaaagaaaaacatatcacggagaaaaaaatgctaatacaacaaataaagatttagatgaattttatagtaaattaaattatttagaaaaaagaagttataaaaatagatatagaaatgataacaataataagaagaataacaataacaataacaataacaataatagtaatagtaacaataattattattattattattcgaatggtcgttcaagttatacaaatgaattatcgaatgaaaaaaataaatataatgaagaaaattcagatgaagggaatgttataaaatataaatatcttccaacaggtgttttttatagtagggtgtcaaaatcttttatagctaattggattgatgataaaacaaaaaaacaagtaaaaattccatataaaatatcagaatatggtatcgagaaatgtatgattttagctatactctcaagaaatctaagactaagtaatttgtcaaatgtattgaaatattatgatgaattgacagatagccaaaaagaacaaatgttacatgcaattagaacaacacagaaaagtgaaaaattatttgaggatattataaataggaatggtaataagaaaaatgaaaataatataataaataattatagtaatattcataatgacacttcaataaataataataataataataataataataataataataatataggaaatacaattggaacacaacataaaaataaaaataaacaaaatgcaaatgatcagaaaaatatacccatgaaaaaaaaacttattgagaaaaaaaaagtcaaacaatcatatgttaatgaagaaaaattaccaacaggtgtatatttttatcaaggttcttatgttgctaattggtgggaaacaaatcaaaaaaaacaatttaaagtacctttcaaaatatccgaatatggaatagccagggcaaaaaatttagccataatttcaagattaattagatcatcttctattccagatattaatctaattttaactcaaatggaaaatgatcataatttgagcgacatggattatactgccatttcggagttggcatataaatatatagaaaatatggctaagaaggaataa

SEQ ID NO: 84 - DNA, 819 bp, Plasmodium falciparum
atgaaggtaattaaaacattgtctattataaatttctttatttttgttacctttaatattaaaaatgaaagtaaatatagcaacacattcataaacaatgcttataatatgagtataaggagaagtatggcagaaagtaagccttctactggtgctggtggtagtgctggtggtagtgctggtggtagtgctggtggtagtgctggtggtagtgctggtggtagtgctggttctggtgatggtaatggtgcagatgctgagggaagttcaagtactcccgctactaccacaactaccaaaactaccacaactaccacaactactaatgatgcagaagcatctaccagtacctcttcagaaaatccaaatcataaaaatgccgaaacaaatccaaaaggtaaaggagaagttcaagaaccaaatcaagcaaataaagaaactcaaaataactcaaatgttcaacaagactctcaaactaaatcaaatgttccacccactcaagatgcagacactaaaagtcctactgcacaacctgaacaagctgaaaattctgctccaacagccgaacaaactgaatcccccgaattacaatctgcaccagagaataaaggtacaggacaacatggacatatgcatggttctagaaataatcatccacaaaatacttctgatagtcaaaaagaatgtaccgatggtaacaaagaaaactgtggagcagcaacatccctcttaaataactctagtaatattgcttcaataaataaatttgttgttttaatttcagcaacacttgttttatcttttgccatattcatataa

SEQ ID NO: 85 - DNA, 819 bp, Plasmodium falciparum
atgaatatattatgtattctatcatatatttattttttcgttattttttatagtttgaatttaaataataaaaatgaaaattttttggttgtcagaagattaatgaatgacgaaaaaggagaaggtggttttacaagtaaaaataaagagaatggaaataataatagaaataatgaaaatgaactaaaagaagaaggttctttacctactaagatgaatgaaaaaaattccaattcatcagataaacagccaaatgatatttcacatgatgaatcaaagagcaattctaataattcacaaaatatccaaaaagaacctgaagaaaaagagaacagtaaccctaatttagatagtagtgaaaattcgagtgaaagcgcaacacgttctgttgatatatcagaacataattctaataatccagagacgaaagaagagaatggagaagaacctttagatcttgaaattaatgagaatgcagaaataggtcaagaacctccaaatagattacattttgacaatgtagatgatgaggtgccacattatagcgccctaagatataataaagtagaaaaaaatgtaaccgatgaaatgttattatataatatgatgagtgatcaaaatagaaaatcatgtgccataaataatggtggatgttctgatgatcaaatatgtataaatataaataatataggagttaaatgtatatgtaaggatggatatttacttggtacgaaatgtataatattgaattcttattcttgccatccatttttttctattcttatttatattacattgtttttgttattattcgtttaa

SEQ ID NO: 86 - DNA, 4677 bp, Plasmodium falciparum
atgacaaatagtaattacaaatcaaataataaaacatataatgaaaataataatgaacaaataactaccatatttaatagaacaaatatgaatccgataaaaaaatgtcatatgagagaaaaaataaataagtacttttttttgatcaaaattttgacatgcaccattttaatatgggctgtacaatatgctaataactctgatataaacaagagttggaaaaaaaatacgtatgtagataagaaattgaataaactatttaacagaagtttaggagaatctcaagtaaatggtgaattagctagtgaagaagtaaaggaaaaaattcttgacttattagaagaaggaaatacattaactgaaagtgtagatgataataaaaatttagaagaagccgaagatataaaggaaaatatcttattaagtaatatagaagaaccaaaagaaaatattattgacaatttattaaataatattggacaaaattcagaaaaacaagaaagtgtatcagaaaatgtacaagtcagtgatgaactttttaatgaattattaaatagtgtagatgttaatggagaagtaaaagaaaatattttggaggaaagtcaagttaatgacgatatttttaatagtttagtaaaaagtgttcaacaagaacaacaacacaatgttgaagaaaaagttgaagaaagtgtagaagaaaatgacgaagaaagtgtagaagaaaatgtagaagaaaatgtagaagaaaatgacgacgaaagtgtagcctcaagtgttgaagaaagtatagcttcaagtgttgatgaaagtatagattcaagtattgaagaaaatgtagctccaactgttgaagaaatcgtagctccaactgttgaagaaattgtagctccaagtgttgtagaaagtgtggctccaagtgttgaagaaagtgtagaagaaaatgttgaagaaagtgtagctgaaaatgttgaagaaagtgtagctgaaaatgttgaagaaagtgtagctgaaaatgttgaagaaagtgtagctgaaaatgttgaagaaagtgtagctgaaaatgttgaagaaagtgtagctgaaaatgttgaagaaatcgtagctccaactgttgaagaaagtgtagctccaactgttgaagaaattgtagctccaagtgttgaagaaagtgtagctccaagtgttgaagaaattgtagttccaactgttgaagaaagtgtagctgaaaatgttgaagaaatcgtagctccaagtgttgaagaaatcgtagctccaagtgttgaagaaatcgtagctccaactgttgaagaaagtgtagctccaactgttgaagaaattgtagctccaagtgttgaagaaagtgtagctccaagtgttgaagaaattgtagttccaactgttgaagaaagtgtagctgaaaatgttgaagaaagtgtagctgaaaatgttgaagaaatcgtagctccaagtgttgaagaaatcgtagctccaagtgttgaagaaatcgtagctccaagtgttgaagaaatcgtagctccaagtgttgaagaaatcgtagctccaagtgttgaagaaatcgtagctccaagtgttgaagaaatcgtagctccaagtgttgaagaaatcgtagctccaagtgttgaagaaatcgtagctccaacagttgaagaaatcgtagctccaacagttgaagaaattgtagctccaagtgttgaagaaatcgtagctccaactgttgaagaaagtgttgctgaaaacgttgcaacaaatttatcagacaatcttttaagtaatttattaggtggtatcgaaactgaggaaataaaggacagtatattaaatgagatagaagaagtaaaagaaaatgtagtcaccacaatactagaaaacgtagaagaaactacagctgaaagtgtaactacttttagtaacatattagaggagatacaagaaaatactattactaatgatactatagaggaaaaattagaagaactccacgaaaatgtattaagtgccgctttagaaaatacccaaagtgaagaggaaaagaaagaagtaatagatgtaattgaagaagtaaaagaagaggtcgctaccactttaatagaaactgtggaacaggcagaagaagagagcgcaagtacaattacggaaatatttgaaaatttagaagaaaatgcagtagaaagtaatgaaaatgttgcagagaatttagagaaattaaacgaaactgtatttaatactgtattagataaagtagaggaaacagtagaaattagcggagaaagtttagaaaacaatgaaatggataaagcattttttagtgaaatatttgataatgtaaaaggaatacaagaaaatttattaacaggtatgtttcgaagtatagaaaccagtatagtaatccaatcagaagaaaaggttgatttgaatgaaaatgtggttagttcgattttagataatatagaaaatatgaaagaaggtttattaaataaattagaaaatatttcaagtactgaaggtgttcaagaaactgtaactgaacatgtagaacaaaatgtatatgtggatgttgatgttcctgctatgaaagatcaatttttaggaatattaaatgaggcaggagggttgaaagaaatgttttttaatttggaagatgtatttaaaagtgaaagtgatgtaattactgtagaagaaattaaggatgaaccggttcaaaaagaggtagaaaaagaaactgttagtattattgaagaaatggaagaaaatattgtagatgtattagaggaagaaaaagaagatttaacagacaagatgatagatgcagtagaagaatccatagaaatatcttcagattctaaagaagaaactgaatctattaaagataaagaaaaagatgtttcactagttgttgaagaagttcaagacaatgatatggatgaaagtgttgagaaagttttagaattgaaaaatatggaagaggagttaatgaaggatgctgttgaaataaatgacattactagcaaacttattgaagaaactcaagagttaaatgaagtagaagcagatttaataaaagatatggaaaaattaaaagaattagagaaagcattatcagaagattctaaagaaataatagatgcaaaagatgatacattagaaaaagttattgaagaggaacatgatataacgacgacgttggatgaagttgtagaattaaaagatgtcgaagaagacaagatcgaaaaagtatctgatttaaaagatcttgaagaagatatattaaaagaagtaaaagaaatcaaagaacttgaaagtgaaattttagaagattataaagaattaaaaactattgaaacagatattttagaagagaaaaaagaaatagaaaaagatcattttgaaaaattcgaagaagaagctgaagaaataaaagatcttgaagcagatatattaaaagaagtatcttcattagaagttgaagaagaaaaaaaattagaagaagtacacgaattaaaagaagaggtagaacatataataagtggtgatgcgcatataaaaggtttggaagaagatgatttagaagaagtagatgatttaaaaggaagtatattagacatgttaaagggagatatggaattaggggatatggataaggaaagtttagaagatgtaacagcaaaacttggagaaagagttgaatccttaaaagatgttttatctagtgcattaggcatggatgaagaacaaatgaaaacaagaaaaaaagctcaaagacctaaattggaagaagtattattaaaagaagaggttaaagaagaaccaaagaaaaaaataacaaaaaagaaagtaaggtttgatattaaggataaggaaccaaaagatgaaatagtagaagttgaaatgaaagatgaagatatagatgaagatatagaagaagatgtagaagaagatatagaagaagataaagttgaagatatagatgaagatatagatgaagatatagatgaagatataggtgaagacaaagatgaagttatagatttaatagtccaaaaagagaaacgcattgaaaaggttaaagagaaaaagaaaaaattagaaaaaaaagttgaagaaggtgttagtggtcttaaaaaacacgtagacgaagtaatgaaatatgttcaaaaaattgataaagaagttgataaagaagtatctaaagctttagaatcaaaaaatgatgttactaatgttttaaaacaaaatcaagatttttttagtaaagttaaaaacttcgtaaaaaaatataaagtatttgctgcaccattcatatctgccgttgcagcatttgcatcatatgtagttgggttctttacattttctttattttcatcatgtgtaacaatagcttcttcaacttacttattatcaaaagttgacaaaactataaataaaaataaggagagaccgttttattcatttgtatttgatatctttaagaatttaaaacattatttacaacaaatgaaagaaaaatttagtaaagaaaaaaataataatgtaatagaagtaacaaacaaagctgagaaaaaaggtaatgtacaggtaacaaataaaaccgagaaaacaactaaagttgataaaaataataaagtaccgaaaaaaagtagaacgcaaaaatcaaaataa

SEQ ID NO: 87 - DNA, 1194 bp, Plasmodium falciparum
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattag

SEQ ID NO: 88 - DNA, 1704 bp, Plasmodium falciparum
atgagaaggtacctgttgattacctgtttgtttgtcctgtgttgcttaaaattaaagcatgtgaactttttaaagtgggagcaggaaaatgatttttattatataaataatgagaaactattaaaaagggtattacataatgtagaacaaactaaagaaagaacagaagttgataaaccaatagtatttggtataaggaaaggaaaatttgttacaatacacaaagaaacaaaagaagagaagatgctgaaggataatttgatagaagctatattatttgatcctaagaaagatgaagaattaaaaattgatataaaagaaacaaatatagataaagatagtaaaaaaaaacaaaaaagagaaaatggaattattaaagatgatacagctaaggataaggatttgtattcatatactaaagacccgattactctccataaaaaaaaattaaaagaagaaaagaattttgttatgatcaaagaatttgtaaaagatttatctagtcgagatgaaaatgtattaatatctaatgtgaacatttttttaaaaagaatatttaatttgatattgagggaaaaaataattactgcaatgtgttcagatgtacaaaatgaaggaatagaaaataataacacacaaatgaagggcaaacaaataaaggacgcacaaatgaagggcaaacaaaataataacacacaaatgaagggcaaacaaaataataacacacaaatgaagggcaaacaaaataataacacacaaatgaatgacgcacaaatgaatgacgcacaaaattatgatggcaaagataacaattcagaatgcttgaaaaataataagaattgtaatttcgataacaaaatcaagattaaagattgtagtaagggttccataagttgttttctctcgaacattaaaaatgaagaattttataaagctccagatttatttaaatattatatatctttagaaaaaatgttgaggagctcttctgttcgatccaaaacagacaggatatcaaaatattttactttttatccagtatctatggataaagaatattatgaagagaaaataaataatcatgtatttttagaggctgttagaaatatattatttgatttagatgaaggaaataaaaaggataaaaaaaaggttttttcgagttttgtaatagtcgtagatacattaatatctttaataaaaaaagaaaaggtagtaaaagaaatgtatatgtttatacatttattttttcaagatttaaatttattaaataaaaaaatattagacattttattaaaaagttcttttaagccaggagcatcatttaatattccagatttcaataagaaaaatttcgaatttattttatcaagaatatatacaagatatgttttaaataatttattaaataagacattcaataattcagataccatcaatatgtctgattttttaaataacaaaataaaacctttcaattttagttttacggaaacaagtgtaaacttgctaaagaatgagggtattcagataaaggatgatgaccttttggtgagcgaagaaaatttgtgtaaatatatacctatcaaaaaaaaattattatatgaaaaacttaacaagacaaggaaagctgcagaggaagctatactggattatatatttagacttttattaagaaaattacatgaatttataacagaataa

SEQ ID NO: 89 - DNA, 474 bp, Plasmodium falciparum
atgaatattcgaaagttcataccatctttagctttaatgcttatattcttcgcttttgcaaacctggtattatcagatgcaaatgacaaagcaaaaaagcccgctggaaaaggatccccttcaactttgcaaaccccaggaagttcttcaggtgcctctcttcatgctgttggacctaatcaaggtggactatctcaaggtctttctggaaaagattctgctgacaaaatgcctttagaaactcagctagctatagaagaaatcaagagcttatccaatatgttagataaaaaaacgacagttaacagaaacttaatcataagtactgctgtcacaaatatgatcatgttgatcatattatctggtatagttggatttaaagttaaaaaaacgaagaacgcagatgatgataaaggagataaggataaggacaaggataatacagatgaaggagacgaaggagatgattcttaa

SEQ ID NO: 90 - DNA, 2520 bp, Plasmodium falciparum
atgcaatcaatggaaataaatgataataacagtatcaagaatgaaagtacatctgatgatgatatattaattaataaaattaaacaaaacttgggtaataataaatcatgtaattctagatcttccaaaaaggaatctataaaaaagcaaaagagcaattctgaacttggtataaaaaagaacacaaagaaatcattaggtataaaaaaagaggaagaaaaaaaaaaacaaataagcaaaagaaaaagtaatgaactaaaagaaaaaaataatttgaaagagggaaaaaagaaatatgtggaaaaaaaatctagaacagtaaaagatgaaaccaagttaacgaatgttataaaaaaagaaactcaaaataataagaaacctaaaaaattacttaaaaaatcagaagaaaattttgaaccaataaatagatggtgggaaaaaatagatgatcaaacagatatacaatggaattatttagaacatcgaggattaatattttcccctccatacgttcaacatcatgtaccaattttttataaaagtataaaaattgaattaaatgcaaaatcagaagaattagctacctattggtgtagtgcaattggtagtgattattgtacaaaagaaaagtttatattaaatttttttaaaacatttataaatagtttagaaaatgataatattataaaacaagagaatgaaacgaaattaaaaaaaggagatatatctaattttaagtttattgattttatgccaatcaaagatcatttattaaaattaagagaagaaaagttaaataaaacaaaagaagaaaaagaagaggaaaaaaaaatgagaatggaaaaagaattaccatatacatatgcgttagttgattggattcgtgaaaagatatcaagtaataaagcagaaccacctgggttatttagaggaagaggagaacatccaaaacaaggtttattaaaaaaaagaatttttccagaagatgttgtaattaatattagtaaagatgcacctgtaccacgattatatgataatatgtgtggacataattggggtgatatatatcatgataataaagtaacatggttagcttattataaagatagtataaatgatcaaataaaatatacttttttatctgctcaatcaaaatttaaaggatataaagatcttatgaaatatgaaaatgctcgaaaattaaaatcatgtgttcataaaattagggaagattataaaaataaaatgaaaaataaaaatattattgataaacaattaggaacagctgtttatttaatagattttctagcattaagagtaggaggagaaaaagatatcgatgaagaagcagatactgtaggttgttgtagtttaagagtagaacatattagttttgcacacgatataccttttaaaagtgtagattcaaaagaacaaaaaacaaatgatgaaaaagtaaataaaataccattaccaacaaatttagaaagtatttcatcagaagattgttatataactttagattttttaggaaaagatagtatacgatattttaatacagtcaaaatagataaacaagcatatattaatataataatattttgtaaaaataaaaatagagatgaaggagtttttgatcaaataacttgttcaaaattaaatgaatatctaaaagaaattatgcctactttatcagctaaagtgtttcgtacatataatgcttcaattacattagatcaacaattaaaaagaataaaagaagtttatggaaaaacaacatattcattatattctggtgaaacagaattacacaaatcgaaaaaaagaaaatctagccatttaacttcagatacaaatatattaagtgatgcaagtgattctactattaatgatgtaaataacgagtatgatgaaaatggaataaataaaaaactatcatatgctactactgtaggaaaagaaaatgatgtcgatgataaaaactcaccaatagaagttgacgtttcaaatataaatgaacttattaatttttacaataatgcaaatagagaagtagccatattatgtaaccatcaaagaagtattccaaaacaacatgatacaactatgtcaaaaataaaaaaacaaattgaattatataatgaagatataaaagaatataaaaaatatttgcaacatttaaaaaaaaatagtgataaaaaatttatctttgtttcgaaagtttctactttagatggaactttaagaccaaataaagtcaaagaaaatatgaaagaagaatcttgtaaaaaaaaactaattactcttataaaaaaagttgaattattaaataaccaaatgaaagtaagagatgataataaaactattgctttaggtacatctaaaattaattatatggatccaagaataactgttgctttttgtaaaaaatttgaaatacccatagaaaaagtatttaatagaagtttaagacttaaatttccttgggccatgtttgctacaaaaaattttacattttaa

SEQ ID NO: 91 - DNA, 546 bp, Plasmodium falciparum
atgagattctccaaagtattttcttttttcgcctttttcattgcccttaaatattttaacagatgccttggtgatcaattagatatgggttccgtacacaataacaattccgttgtaggaaactcatcatcacattcaccatcatcatcatcatcttcaccatcttcttcttcttcttcatcatcatcatcaccatctgcatcttcatcttcatcctcatcatccccagcttcctcatcttcaagcccatcaagcacatcagatgacagcaaaaatgcatccttagataaaatcgatgaagaactccaaaagaaaaagaaaaacgaaaaattacttttaatatcctctattgccacaggtttagccgttttagtaggtggaataataggtactgctttatacgcaagcagaaaatcatcaaaagcctgcaaaaataacgttgacgatttagattcagatgttgaagaagccgatgttaccgaggaatcagctgttgaaaccaagaaagacgaagtcaaaaccgaagaacccaaaaaagaacaataa

SEQ ID NO: 92 - DNA, 1242 bp, Plasmodium falciparum
atgaaccttttagtttttttttgttttttccttttatcgtgcatagtccatctttcaagatgttcagataataacagctactcatttgaaattgtgaatagatctacgtggttaaatatagcagagagaatattcaaaggaaacgctccatttaattttacaataattccatataattatgtaaataattctacagaagaaaataataataaagattcagttttattaataagtaagaatttaaaaaattcttccaatcctgttgatgaaaataatcatataattgacagtacaaaaaaaaacacatcgaataataataataataatagtaatattgttgggatatacgaatctcaagtacatgaagaaaagataaaagaagataatacacgtcaggataatataaataaaaaggaaaacgaaataataaataataatcatcaaataccagtatcaaatattttttcagaaaatattgataataataaaaattacattgaatcaaattataagagcacgtataataataatccagagttgattcattcaacagattttattggttcaaataataatcatacatttaattttctttcgagatataataatagtgtattgaacaatatgcaaggaaatacaaaagttccaggtaacgttcctgaattaaaagctagaattttttcagaagaagaaaatactgaagtagaatctgcagaaaataatcatacgaattcattaaaccccaatgaatcatgtgatcaaataataaaattaggtgatataataaatagtgtaaatgaaaaaatcatatctataaattcgacagtaaataatgtattatgtataaatttagattcagtaaatggaaatggttttgtatggactttattaggagtacataagaaaaaaccattgattgatccatctaatttccctacaaaaagagtaacacaatcatatgttagtcctgatatttcagtgaccaatccagtacctatacctaaaaatagtaatacgaacaaagatgattcaataaataataaacaagatggaagtcaaaataacaccacaacaaatcattttcctaagcccagagaacagctagtcggaggctcatccatgttaataagtaaaattaagccccacaaacctggaaaatattttattgtctattcatattatagaccatttgatccaacaagggatacaaacacaagaattgtagagttaaacgtgcaataa

SEQ ID NO: 93 - DNA, 918 bp, Plasmodium falciparum
atgctttccatagcaagtaccttcggatcttatttttttagttcttgttcagtagattatttgaataatgaaagtgcaaacaaattttcttcatctgatgaaaagataaattttaggagaaaaagaaatgtatcatcttcatcaaaggatgataaaatcaatattaataatgaccatgataaaattttaaatgaaagtgatgattcacatgatattaacaaaaatttagataatttggaatatgcaacacaaaataataataaaggaaatatatatgaagaagaacatatagggatcaataatgaaaaagaaaaaaacattgatcatcatcaagaagttcaaagtaaagattcacatgacaatcaaaatgaacatgaacaaaattatatgaaatctaatgagaatacacttccaaatgatcatgagaaatcgaatgagaatgcacttccaaatgatcatgagaaatcgaatgagaatgcacttccaaatgatcatgagaaatcgaatgagaatgcacttccatatgataatatgaaattagataaagatgaatatcctaatccacctataaaatctcatgaacaagattgttgttctgataaaagttttgatgaatgtgcaagaaaaaaagaattggaacttaataataataaaaaaaattatgacatatataatgaaaatgcagaagaatccaataattatagcagcccatataatgaaaaaaaatatagactaataggagatttttcaagatatatgtctgttacaataaatgaaaaaaaaggtggtcttcataataattgtacaactgtattagtagatgttgatttatttcctaacgtttcagaaaattatttatcaaaaatgtttgattatttaacaaattttaaagatgaggaaataaacaaaaatgaataa

SEQ ID NO: 94 - DNA, 4242 bp, Plasmodium falciparum
atgaacggttccttttttttttctcattttaaagaattcacaataagaaatacaaattatttcttgtatgaagaatcaaaaaagagttataaaagttgtattgtatggtatatgaaccattatcaacgttttggtggatcgataagaagatattcaaatgcattggggcaattaattgaccggaaaaaaagaaacttggtaaatatagaaaagggtaatgaaattttgaaaagttataaaagagttaacttaaagaagatattgaaggatggtgataaaaaaaaggaaatttacgaaaatgataaaattgtaagtgataatattataagtgataataatataagtaataataatataagtgataataatataagtcataataatataggtgataataatataagtaataatattataagtgataatattttaagtgacaatattttaagtaataatattttatgtgacaatattgttaatgacaagattaagtatgataaaggggaaaagtggataaatgaaaatgtgttgaaaaaagtggaaggaaaagataatgagaatttggatgattttaatgataaggataagaggaataataataaaaatattatgaatatagaaaataagaatatttataatataaatacaaatagtagtacattttgtacacataatcatataaatgatgaacatgtaaaagattcggtggatgaattattttgtacaaaagaaaagaaaaaacaattaatgaaaagaattatatttcagaataatcatgattatatgcatttaattgaaagatacgaagaatataaaataaaacaagaagatgaattttataattcgagtaatgataaaaatgaggaggaatactcatcttacccttcttttattaaaaaaaaagaagaaaataaaagtaatgatgataacaataataatgtaaatgaaaaaatgtgtgaaaaagaaaattttaagtgtaaagaaaagagtagtaataatgatgtacattattatttatatgaagtggataaagataatgaagaatctaatgtaaatatgcattttaattttttaaaaaacaatttaaacatttctatatttcccgagataggaaagaaggatatattagaaagttgtaaacgtaagaagaatatgcaaaatgaaataaacaaaaatagtgatataaaaaatatgtttaataatgatatttatttttggacgaaaaggaaagttcatcgagctataggttgggatttaatcccagataatttgaaaaggaatgatgatataatcgatgtgtcagagttgagatatggtcatttaaaaggctctacaaaagaagaggaaaaggaagataataatagagaggtaggatataaagatgataataataagattcataaaaccatgaagcaacatattaataaggatatagaacaaaacacaggacaagataaaaattctaatataaaaggtaaatctgttgaagaggaaaatatgtacatgggagaaaagaaaaatgacataaatgtacaacttgatgatataaatgtacaacttgatgatataaatgtacaacttgatgatataaatatacaacttgatgaaataaatttaaataaggacgaacgtgataaaaaaaaaattagttacatacaaaggagggttgcaccttgcaattttttaaaaaaattggatatatataaaagtgatgcaacccttttaaatgatggttcttctactattcaaatgaacaaaaatgtagatgtgatttgtgaggaggataacaaagaaatggagaaagaggaagaaaaaaaatatgaagaaacaaacaaagaaccaacgaaccaaatgaaggaagaaaaattaattgatatatttttaaataatgataacaacttgggtaatgtcaaaaacgtggaatatattaaggctgaggtaaataaaggagaccataagttggacaagttagatataaaaaaagaggatgatgaatataacgaaattgtagagattgatagttgtcagaatgataatataaaaaataaggagaagaatatgaaatatatgaatggtagctataataatatgaatgaagaggataagcattatgaatctgatgataagacatattatgaagggattgttgataatagtaagaacgtattcaaaaagatgaaggagaattatgatatgtttaagaagaataatatgtcaccgtatatattagaagattgtgaaaagtatataaattattattatggtatagagaaggaagaaaggattaaagagctgagaaaatatgctgatatggatttttacgagataatgggaaatggtaattttggtatagaggattataatatgttaattaagagtaagatattatttaataaagaagaggaaggttttcattattttaatttattaaaaaaatatgatataagaataaatatagaaacgtataatagtttaatgtatacatgtatagtacaaaagaattcaaaattgagtaggttaatatatttgcagataataaaagatttatttatacctaataagaatacattttgtatattaataaaagctcacatattggataaggatataaagagtgcatttcatttgtataggaaaatgataaaagagaatattgaagtagatatagtaatttattcaacattaatagatggattaataaaaaataaattatataaacgagcagaacagttttttaattatatagttaattataaaaatgtagtaccagatgaaatattatatacaataatgataaaaaattgtgcgtataatagagaagcagaaaaatgtctaaattattacgaaactatgttatctcaaaatttaaggataaccgatataacattaatagagataattaattgtttatcaaggagggaagattatttttataaagtgttttatttttatcatatttatttatctaatgagatgaaaataaatcaacgtttaatgttatatatgataatggcatgttcaaataaaggaaatataaaaagattaaaggagatattaaagactatgaataaaaataaaatcaaaataagtgatgaaatgtattgttatattgttagaacgtttgcaaataattgtaaggataagcgtgttagcttaagtgagcgtcataataatataaagtatgcatggaggattatatatgacttattgaaaggttcttcccatatgaaaaaaaaagaaaaggaaaaagaaaaagatatatatatagataaatatatagataaggaaaaagaaaaatatatagataaatatatagataaagaaaaagatatatatatagataattatatagataaacaaatcgatgagagacatggcgagatgataagtacaaaggaacatataaaaaagaaccctactgatgataataaaaatattgagagttacaacaaaaatgatcatatctcttttgatagagttaatcatttgtataaaggtaccgagttacattcttctacatcacatgattgtataaacaccaaaatattaaacagtcttattttattatatataaattgtgaatattatgaatatgctataaatatgttgaaatatttttcatattttgaatgtgtaccagattattatactttcaatatgttattcaatatgttatattacaaaatgaaggattatggtaaggtgttatgtttatatgattatatgataaataacacacaaaacaaacctaatgaaaaaatattgaacttaatattaaatagcgctatacaaacaaaatcatcaaaaaatactttgtttatattacgtcagatgtttacatataaaatatatccctctcctaaaatggtaaaaaaattatatcatgtgggaagatatataacggaaatacaattattaattaatagtatgatacgtcaacaaacgaaagatatatatgaagtaaatttgaaggaaaatcagctaatacgattaaatatagatgagtacgaattaaatttatttaaagaaggaaaaacattcaaaagtaaaactcctttagatgaagccagagaacaattctttaaacgaaagcaaagaatggaaaaggaaaaaagaatgtctaaaaataaaaaatcttcagattggttaccatatggtcaatatttacaaagtaaaaagaagggtggtgaaatctatgcaaaaagggtcgataggcctcgacctttagcatttgacgactga

SEQ ID NO: 95 - DNA, 3120 bp, Plasmodium falciparum
atgaagaatgaaacacaacattttttcatcgtatgtttattcgacatagcattttttgcggtgtgtgtttgtatatggttatatttacgaaagaaccgcgatgaaaataagataaatgataacatatatatattaaatcaccagacgcatagtgaaataaagatagataataaagagactgtgggtgtaacgaatgatgatagtactacagaagtaaagaagaaaacgaatagatttcgtacatttttaaatataaaagatgataaaatagtaaatacggaaataaaatattatttattttttttgaaagcaaatagaaatattatattttctttatgtattttaggaacctttttagtgttaccattatatttatcattaccaaggaatgatattaataatccatctttttttcattatattagtgctggtagtatatcggatataaatatattaactatattatattttattactttaatatatagtgctatatcgtatgcatttatttatttattatggagaaaaattcgacctagtaaaaagaaaaccaaaaaattccttcctcagaattttactattatggtatctcgtatagataaaaaagaaattaatgcttataaaatatatgagtatttttgtaacctaacaaataataaagtggtctcagcgtatcttatattagattattcaatagtgtatcatgagcagaaaaaaatttttaacgcaaccaaaaatcttaaattacttaaagagaatgaagagaaaaaaaatattaaaagagataaatcgaagaatatgtttttttcttggacatctaaaaaaaaaaagaaaaaaaacgcatcaaatgaccccctttctatttgtgataaaaatacaaatcaaaatttaaaaatctcaaaaaataatacgacagaaggtaatcatatgcataattctgtgtctatgaaaaaatcgaagcatgcctttgtcgaaaataataatacaacaggaaactataaaacaaaaagtgatcacaacaaattaagtagtaatgataatattaaaaattttaatgaagataaaaagaaaaagaagcatcacaatcaaaatcaaaataacaatcaaaatgatgcatatagtaatgcttccaaaggtgtaaccatacaagcaaatgacaaaaatgtgaacagtgatataaatacaagtatagaaaagaggatcaaaaagggtacacacaaaagtaacgaaatatacaaacaaaaatgtagtagcaaagtgaaggaaaatcaaatattgggaaaaaaagaaaaagaaaaaaatattaaagaggaacctcaaagtgatgctcttgataaggatgataataaagaagatgataataatgatgatgataataatgacaatcatcataataatgatgataataatgacgatcatcataatgatgatgatgataataataacgatcatcataataatgatgatgataataatgacgatcatcataatgatgatgaaaatatggaaaatatacaatccaaaagagataatgataatgtagaagaagaacctgcttgttttttaaaagacaccacagatgaagagttagtagtggaaaaaggtaaattacgaaaaaagaaaatagcaggttcaaattatggttacaaacttgatttagaagaaaaattaatgagaagcacaaacaatgatatgttattttatgatttggataatttatcaacaaaagataatgataatattgatataaaatataaaaaaaagaagaagaaaaatatatataaaagtgaatcgaagacaaacttttttgataaaatatttttctttttaaaaaaaaataaaaaaagtcattggaaaaaaaaattaaaagaacatttaattaaattttattcaataaaaaatgaaactccaaaaaaatctacaggagtatgttttgtatcctttatagataccaaatcagtacatgattgtatacacaatataccttttacggaaagaaataaatgggtaatatctaatgcaccacctaattatgatattatatggaaaaatctaaaaaatgatagttataaagtatgtgctcgttttattatattaaatgcattactattattagctaatacaataatgattatatctgtaacatccattgataatatcttaaaactcaaaatcaaaaaatataaagcagcagaccctagctcatccaatcttagtgctattttgacaacttggttatccccatttattgttatatttgtgaacagtattattcaaccagctttaatttcgtgcgtatctatgattattggatttataagaaagtcaagtgaacacacgtacgtgttacaaggaaattttatctttttaattttaaacacaattattatacctttgctttcgctttctcccttaagttcgataataaaggtcatgtattctgatgaaatcggacagtggtcaacacgtctgggagaatatctttttaactctagtgggttctttgctatgcgttatttacttcattgttgtttcttaacgtgcgcaaatcagttattgcaaattccacaattttcaatacggtcaatttttaagattcttacgaaaaaagaaatcagtgcgtggacttttgattttggatattggtatggatttaatacttcaatattagccttgatattgacctttagtgttgctgtgcctttcatattaccactagggtccctatactttttcttgcgatattatatcgacaaatacaatttaatttatgaaatatgtcgaacaaatttggatagtcacggagcagtagttagaacggcgataaaatttatgcttttttcagtggccttctttcaattggttatgttcacattcttttcgagggttcagaataagttcatatcagtcggccgaaacatattatttttgtcatcttccttgacaacacttttattattgtgtagatcaacagaatgggttagcaccaatcacataaaaagaaaaaaagggaaacggaccttttgttatttatgtgaaaaaaatgtttatgttagtaatttaaaagatttaaacaaattaaaatatgcttatgccaatccttgtgaatcaaagagataa

SEQ ID NO: 96 - DNA, 2178 bp, Vaccinia virus
atggaggtcacgaaccttattgaaaaatgtaccaagcactccaaagatttcgccactgaggtaaaaaaactatggaacgatgagttgagttctgaatcaggtctctcaagaaaaacaagaaatgtaattcgtaatattcttcgtgatatcactaagtcattaactacagataagaaatcaaagtgtttccgtatactagaacgttcgacgattaacggagagcagattaaagatgtatataaaactatttttaataatggtgttgatgtggagtctagaatcaacactacaggaaagtatgttctatttacagttatgacttatgttgctgctgaactacgactcattaagtcagacgagatattcgctcttctatcaagattttttaacatgatatgtgatattcatagaaaatacggatgtggtaatatgtttgttggtattcccgctgctctaattattctgttggaaattgatcacatcaataaactgtttagcgtgtttagtacaagatatgatgctaaggcatatctatatactgaatatttcctcttccttaacattaatcattatctacttagtggttcagatttatttatcaacgtagcatatggtgctgtatctttttcgtcacccattagtgttccagattatatcatggaagcactgacatttaaggcatgtgatcatattatgaaatctggagatctaaaatatacatatgcgtttactaaaaaggttaaggatctgtttaatactaaatctgattctatttatcaatacgttagacttcatgaaatgtcatatgatggtgtttcagaagatacggatgatgacgatgaggtattcgctatccttaacttgagtattgattccagcgttgatagatacagaaacagagttcttctactaactcccgaagtcgcgtctcttagaaaagaatattcagacgtagaacccgattataaatacttgatggatgaggaagtgcccgcgtacgacaagcatttgcctaagcctattactaacactggtattgaagaaccacacgctactggaggagatgaggaccaaccaattaaggttgtccatccccctaataatgataaagatgatgctatcaagccatacaatccattagaagatcctaattatgttcccacaattacaagaacggctataggaatcgctgattaccaactagttattaataaactaattgaatggttagataaatgcgaggaagaatgcggaaatagtggagagtttaaaacagagttggaagaagccaagagaaaactcaccgaattgaatgcagaacttagtgataaactcagtaagattaggactttggaaagggattctgtttataaaaccgaaagaatcgaccgacttacaaaagagatcaaagaacacagggatattcaaaatgggacagatgatggttcagatttattagaaattgataagaagactatccgagaattgagagaatcgcttgatagggaacgagaaatgcgttcagaactagaaaaggaactggatactattaggaatggaaaagtagatggatcttgtcaacgagaacttgaactcagtcgtatgtggctaaaacaacgcgatgacgatctccgagctgaaatcgataaacgtcgtaatgtcgaatgggaactgtccagacttcgtagggatatcaaggaatgcgacaaatacaaggaggatcttgataaggccaagacaactattagtaactacgtaagcaaaatcagtactctagaatcagaaattgctaaatatcaacaagatagggacacgctttctgtagtacgcagagaacttgaggaagaacgacgacgcgttagagatctcgaatctagactcgatgaatgtacacgcaaccaggaagacacgcaagaagttgatgcactgcgttcgcgtattagagaactagagaataagttgaccgactgcatcgagagcggaggaggaaatcttacagagattagcagactccaatctaaaatctcagatcttgaaagacaactgagtgaatgccgtgaaaatgctacagagattagcagactccaatctagaatatcagatcttgaaagacagttgaacgactgtagacgtaataatgaaaccaatgccgaaacagagagagatgcgacgtcttag

SEQ ID NO: 97 - DNA, 819 bp, Plasmodium falciparum
atgtggatagttaaatttttaatagtagttcatttttttataatttgtaccataaactttgataaattgtatatcagttattcttataatatagtaccagaaaatggaagaatgttaaatatgagaattctaggggaagaaaaaccaaatgtggacggagtaagtactagtaatactcctggaggaaatgaatcttcaagtgcttcccccaatttatctgacgcagcagaaaaaaaggatgaaaaagaagcttctgaacaaggagaagaaagtcataaaaaagaaaattcccaagaaagcgcgaatggtaaggatgatgttaaagaagaaaaaaaaactaatgaaaaaaaagatgatggaaaaacagacaaggttcaagaaaaggttctagaaaagtctccaaaagaatcccaaatggttgatgataaaaaaaaaactgaagctatccctaaaaaggtagttcaaccaagttcatcaaattcaggtggccatgttggagaggaggaagaccacaacgaaggagaaggagaacatgaagaggaggaagaacatgaagaagatgacgatgacgaagatgatgatacttataataaggacgatttggaagatgaagatttatgtaaacataataatgggggttgtggagatgataaattatgtgaatatgttgggaatagaagagtaaaatgtaaatgtaaagaaggatataaattagaaggtattgaatgtgttgaattattatccttagcatcttcttctttaaatttaatttttaattcatttataacaatatttgttgttatattgttaataaattaa

SEQ ID NO: 98 - DNA, 4389 bp, Plasmodium falciparum
atgaaatgtaatattagtatatatttttttgcttccttctttgtgttatattttgcaaaagctaggaatgaatatgatataaaagagaatgaaaaatttttagacgtgtataaagaaaaatttaatgaattagataaaaagaaatatggaaatgttcaaaaaactgataagaaaatatttacttttatagaaaataaattagatattttaaataattcaaaatttaataaaagatggaagagttatggaactccagataatatagataaaaatatgtctttaataaataaacataataatgaagaaatgtttaacaacaattatcaatcatttttatcgacaagttcattaataaagcaaaataaatatgttcctattaacgctgtacgtgtgtctaggatattaagtttcctggattctagaattaataatggaagaaatacttcatctaataacgaagttttaagtaattgtagggaaaaaaggaaaggaatgaaatgggattgtaaaaagaaaaatgatagaagcaactatgtatgtattcctgatcgtagaatccaattatgcattgttaatcttagcattattaaaacatatacaaaagagaccatgaaggatcatttcattgaagcctctaaaaaagaatctcaacttttgcttaaaaaaaatgataacaaatataattctaaattttgtaatgatttgaagaatagttttttagattatggacatcttgctatgggaaatgatatggattttggaggttattcaactaaggcagaaaacaaaattcaagaagtttttaaaggggctcatggggaaataagtgaacataaaattaaaaattttagaaaaaaatggtggaatgaatttagagagaaactttgggaagctatgttatctgagcataaaaataatataaataattgtaaaaatattccccaagaagaattacaaattactcaatggataaaagaatggcatggagaatttttgcttgaaagagataatagatcaaaattgccaaaaagtaaatgtaaaaataatacattatatgaagcatgtgagaaggaatgtattgatccatgtatgaaatatagagattggattattagaagtaaatttgaatggcatacgttatcgaaagaatatgaaactcaaaaagttccaaaggaaaatgcggaaaattatttaatcaaaatttcagaaaacaagaatgatgctaaagtaagtttattattgaataattgtgatgctgaatattcaaaatattgtgattgtaaacatactactactctcgttaaaagcgttttaaatggtaacgacaatacaattaaggaaaagcgtgaacatattgatttagatgatttttctaaatttggatgtgataaaaattccgttgatacaaacacaaaggtgtgggaatgtaaaaaaccttataaattatccactaaagatgtatgtgtacctccgaggaggcaagaattatgtcttggaaacattgatagaatatacgataaaaacctattaatgataaaagagcatattcttgctattgcaatatatgaatcaagaatattgaaacgaaaatataagaataaagatgataaagaagtttgtaaaatcataaataaaactttcgctgatataagagatattataggaggtactgattattggaatgatttgagcaatagaaaattagtaggaaaaattaacacaaattcaaattatgttcacaggaataaacaaaatgataagctttttcgtgatgagtggtggaaagttattaaaaaagatgtatggaatgtgatatcatgggtattcaaggataaaactgtttgtaaagaagatgatattgaaaatataccacaattcttcagatggtttagtgaatggggtgatgattattgccaggataaaacaaaaatgatagagactctgaaggttgaatgcaaagaaaaaccttgtgaagatgacaattgtaaacgtaaatgtaattcatataaagaatggatatcaaaaaaaaaagaagagtataataaacaagccaaacaataccaagaatatcaaaaaggaaataattacaaaatgtattctgaatttaaatctataaaaccagaagtttatttaaagaaatactcggaaaaatgttctaacctaaatttcgaagatgaatttaaggaagaattacattcagattataaaaataaatgtacgatgtgtccagaagtaaaggatgtaccaatttctataataagaaataatgaacaaacttcgcaagaagcagttcctgaggaaagcactgaaatagcacacagaacggaaactcgtacggatgaacgaaaaaatcaggaaccagcaaataaggatttaaagaatccacaacaaagtgtaggagagaacggaactaaagatttattacaagaagatttaggaggatcacgaagtgaagacgaagtgacacaagaatttggagtaaatcatggaatacctaagggtgaggatcaaacgttaggaaaatctgacgccattccaaacataggcgaacccgaaacgggaatttccactacagaagaaagtagacatgaagaaggccacaataaacaagcattgtctacttcagtcgatgagcctgaattatctgatacacttcaattgcatgaagatactaaagaaaatgataaactacccctagaatcatctacaatcacatctcctacggaaagtggaagttctgatacagaggaaactccatctatctctgaaggaccaaaaggaaatgaacaaaaaaaacgtgatgacgatagtttgagtaaaataagtgtatcaccagaaaattcaagacctgaaactgatgctaaagatacttctaacttgttaaaattaaaaggagatgttgatattagtatgcctaaagcagttattgggagcagtcctaatgataatataaatgttactgaacaaggggataatatttccggggtgaattctaaacctttatctgatgatgtacgtccagataaaaatcatgaagaggtgaaagaacatactagtaattctgataatgttcaacagtctggaggaattgttaatatgaatgttgagaaagaactaaaagatactttagaaaatccttctagtagcttggatgaaggaaaagcacatgaagaattatcagaaccaaatctaagcagtgaccaagatatgtctaatacacctggacctttggataacaccagtgaagaaactacagaaagaattagtaataatgaatataaagttaacgagagggaaggtgagagaacgcttactaaggaatatgaagatattgttttgaaaagtcatatgaatagagaatcagacgatggtgaattatatgacgaaaattcagacttatctactgtaaatgatgaatcagaagacgctgaagcaaaaatgaaaggaaatgatacatctgaaatgtcgcataatagtagtcaacatattgagagtgatcaacagaaaaacgatatgaaaactgttggtgatttgggaaccacacatgtacaaaacgaaattagtgttcctgttacaggagaaattgatgaaaaattaagggaaagtaaagaatcaaaaattcataaggctgaagaggaaagattaagtcatacagatatacataaaattaatcctgaagatagaaatagtaatacattacatttaaaagatataagaaatgaggaaaacgaaagacacttaactaatcaaaacattaatattagtcaagaaagggatttgcaaaaacatggattccataccatgaataatctacatggagatggagtttccgaaagaagtcaaattaatcatagtcatcatggaaacagacaagatcgggggggaaattctgggaatgttttaaatatgagatctaataataataattttaataatattccaagtagatataatttatatgataaaaaattagatttagatctttatgaaaacagaaatgatagtacaacaaaagaattaataaagaaattagcagaaataaataaatgtgagaacgaaatttctgtaaaatattgtgaccatatgattcatgaagaaatcccattaaaaacatgcactaaagaaaaaacaagaaatctgtgttgtgcagtatcagattactgtatgagctattttacatatgattcagaggaatattataattgtacgaaaagggaatttgatgatccatcttatacatgtttcagaaaggaggctttttcaagtatgccatattatgcaggagcaggtgtgttatttattatattggttattttaggtgcttcacaagccaaatatcaaaggttagaaaaaataaataaaaataaaattgagaagaatgtaaattaa

SEQ ID NO: 99 - DNA, 1056 bp, Plasmodium falciparum
atgaagagtaatatcatattttatttttcttttttttttgtgtacttatactatgtttcgtgtaatcaatcaactcatagtacaccagtaaataatgaagaagatcaagaagaattatatattaaaaataaaaaattggaaaaactaaaaaatatagtatcaggagattttgttggaaattataaaaataatgaagaattattaaacaaaaaaattgaagaattacaaaacagtaaagaaaaaaatgtacatgtattaattaatggaaattcaattattgatgaaatagaaaaaaatgaagaaaatgatgataacgaagaaaataatgatgatgacaatacatatgaattagatatgaatgatgacacattcttaggacaaaataacgattcacattttgaaaatgttgatgatgacgcagtagaaaatgaacaagaagatgaaaacaaggaaaaatcagaatcatttccattattccaaaatttaggattattcggtaaaaacgtattatcaaaggtaaaggcacaaagtgaaacagatactcaatctaaaaatgaacaagagatatcaacacaaggacaagaagtacaaaaaccagcacaaggaggagaatcgacatttcaaaaagacctagataagaaattatataatttaggagatgtttttaatcatgtagttgatatttcaaacaaaaagaacaaaataaatctcgatgaatatggtaaaaaatatacagatttcaaaaaagaatatgaagacttcgttttaaattctaaagaatatgatataatcaaaaatctaataattatgtttggtcaagaagataataagagtaaaaatggcaaaacggatattgtaagtgaagctaaacatatgactgaaattttcataaaactatttaaagataaggaataccatgaacaatttaaaaattatatttatggtgtttatagttatgcaaaacaaaatagtcacttaagtgagaaaaaaataaaaccagaagaggaatataaaaaattcttagaatattcatttaatttactaaacacaatgtaa

SEQ ID NO: 100 - DNA, 465 bp, Vaccinia virus
atggaaacaatcaaagcgttggagaaatttatggagttcgatcgccttcagaaagactgctctgataaactcgatagagagaaggagagacgcatgaaggctgaacgtgaaatcgctcgtaaaaactgcggaggtaacccatgcgaacgtgaattggaatctgaacgtagtaacgtgaagaggttggaatatcaactagatgctgagaaagaaaaagttaagttctacaaaagagaactagaacgtgatcggtatctttctagtagatatcttacctcttcttcagatccacatgagaaaccattaccaaattatacatttcctcgcattaaaaatgtatctccgttgacaactgaggctacaggttctgtagaagtagcacctccatccacagacgttaccgaaccgattagtgatgtgacaccatcggtggatgtcgaaccagaacatcccccagctttctga

SEQ ID NO: 101 - DNA, 1503 bp, Vaccinia virus
atggcgaacattataaatttatggaacggaattgtaccaacggttcaagatgttaatgttgcgagcattactgcgtttaaatctatgatagatgaaacatgggataaaaaaatcgaagcaaatacatgcatcagtagaaaacatagaaacattattcacgaagttattagggactttatgaaagcctatcctaaaatggatgagaataaaaaatctccattaggagccccaatgcaatggctaacacaatattatattttaaagaatgaatatcataagaccatgctagcgtatgataatggatcattgaatacaaaatttaaaacgttaaacatttatatgattactaacgttggtcaatatattttatatatagtattttgtataatatctggtaagaatcacgatggtactccttatatatacgattctgagataacgagcaatgataaaaattttattaatgagcgtatcaagtatgcatgtaagcaaatattacacggtcaattaactatagctctgagaattagaaataaattcatgtttataggatcacccatgtatttatggtttaacgtaaacggatcacaggtatatcacgacatatatgatcgtaatgccggttttcataataaagagataggtagactactatacgcatttatgtactatctatctataagtggtagatttttgaatgatttcgcactattaaagtttacgtatttaggagaatcctggacatttagtttgagtgtccctgaatatatattatatggtttaggatattctgttttcgatactattgaaaaatttagcaatgatgctatactcgtttatattagaacaaacaatagaaatggatatgattatgtagagtttaataaaaaaggaattgctaaggtgacagaagataaacccgataacgataagcgaattcatgctataagactcatcaacgatagtactgatgttcaacacatacattttgggtttagaaatatggtaataatagacaatgaatgcgctaatattcagtcgagtgctgaaaatgcaactgatacaggacatcatcaagatagcaaaataaatatcgaagtcgaagatgatgtcatagacgatgatgattataatccaaaacccactccgataccggagcctcaccctagaccaccgtttcccagacatgaatatcataagaggccgaaacttcttcctgtagaagaacctgatcctgtcaaaaaagacgcggatcgtataagacttgataatcatatattaaacacattggatcataatcttaatttcatcggacactattgttgtgatacagcggcagttgataggttagaacatcacatcgaaacattgggacaatatgcagtaatactggcaagaaagataaatatgcaaacattactgttcccatggccattacctactgtccatccacatgcgatagatggtagtattccaccacatgggagatctacgattttataa

(19) United States
(12) Patent Application Publication
(10) Pub. No.: US 2018/0016299 A1
(43) Pub. Date: Jan. 18, 2018

(54) METHODS FOR MAKING ARRAYS FOR HIGH THROUGHPUT PROTEOMICS

(71) Applicant: The Regents of the University of California, Oakland, CA (US)

(72) Inventors: Philip Felgner, Rancho Santa Fe, CA (US); Huw Davies, Irvine, CA (US); Xiaowu Ling, Irvine, CA (US)

(21) Appl. No.: 15/472,234

(22) Filed: Mar. 28, 2017

Related U.S. Application Data
(63) Continuation of application No. 11/571,034, filed on Jun. 4, 2008, now abandoned.
(60) Provisional application No. 60/638,624, filed on Dec. 23, 2004.
(60) Provisional application No. 60/585,351, filed on Jul. 1, 2004.

(51) Int. Cl. C07K 1/107; A61K 39/02; A61K 39/04; C07K 14/005; A61K 39/015; A61K 39/12; A61K 39/285; C07K 14/195

(52) U.S. Cl. CPC C07K 1/1077; A61K 39/285; A61K 39/0208; C07K 14/195; C07K 14/005; A61K 39/015; A61K 39/12; A61K 39/04; C12N 2710/24143; C12N 2795/10222

(57) ABSTRACT

Methods to obtain expression systems and proteins in a high-throughput protocol by utilizing mixtures of cells cultured from those transformed with a desired nucleotide sequence permit rapid production of protein for use in arrays to assess activity. In one embodiment, the proteins (or peptides) in the array are assessed for their immunological activity with regard to an infectious agent.

### CROSS-REFERENCE TO RELATED APPLICATIONS

This application is a continuation of and claims benefit of priority under 35 U.S.C. §120 to U.S. patent application Ser. No. 11/571,034, filed Jun. 4, 2008 (currently pending), which is a national phase of International patent application serial number PCT/US2005/023352, filed Jul. 1, 2005, which claims benefit of priority under 35 U.S.C. §119(e) of U.S. provisional application 60/585,351 filed 1 Jul. 2004, and U.S. provisional application 60/638,624 filed Dec. 21 23, 2004. The contents of each of these applications are incorporated herein by reference in their entirety and for all purposes.

### STATEMENT OF RIGHTS TO INVENTIONS MADE UNDER FEDERALLY SPONSORED RESEARCH

This work was supported in part by National Institutes of Health/National Institute of Allergy and Infectious Diseases. The U.S. government has certain rights in this invention.

### TECHNICAL FIELD

The invention relates to methods to generate proteins or peptides from encoding open reading frames (ORF) and to methods to identify immunologically active proteins. The invention also relates to methods to generate protein/peptide arrays from a multiplicity of encoding ORF's and to the use of such arrays to determine immunologically active proteins. It also relates to these immunoactive peptides and methods using them.

### BACKGROUND ART

It has long been known that microorganisms such as E. coli and yeast contain recombinase systems that effect homologous recombination without the necessity to supply extraneous enzymes such as ligases. For example, Oliner, J. D., et al., Nucleic Acids Res. (1993) 21:5192-5197 describe methods to clone PCR products by providing them with terminal sequences identical to sequences as two ends of a linearized vector. The products and vector DNA were cotransfected into E. coli strain JC8679 and the vector and PCR products were recombined in vivo. Colonies containing recombinant plasmids were identified by hybridization to diagnostic DNA. The authors suggest an optimized protocol for cloning genomic PCR products in E. coli using this method.

More recently, Zhang, Y., et al., Nature Genetics (1998) 20:123-128 described a similar approach which was stated to enhance the size of the DNA that could be cloned in this manner.

U.S. published application 2003/0044820 describes a method for cloning a nucleic acid fragment into a vector using PCR by employing adapter sequences which may contain functional elements such as promoters, terminators, selection markers, and the like. The linearized vectors were amplified by PCR rather than preparing the linearized vector by cloning and then digesting as conventional. This has the added advantage of providing additional sequences to the linearized vectors which may match the attached portions of the

PCR amplified nucleic acid. A unique system for selecting colonies with recombined plasmids is also described.

More recently, Parrish, J., et al., J. Proteome Res. (2004) 3:582-586 describe parameters that affect cloning efficiency in employing the general technique of recombination in E. coli. In this work, reading frames identified in Campylobacter jejuni were amplified and inserted into linearized vectors in E. coli. Individual colonies were isolated and the clones sequenced. Primer pairs to amplify full-length ORF's for the 1,685 genes predicted for the genome sequence which had already been determined for this organism were used. 1,346 PCR products were visible on a gel and 75% of these provided colonies that had the vector with an insert.

It is also known that cells other than E. coli exhibit recombinase functions. For example, Ma, H., Kunes, S., Schatz, P. J. and Botstein, D., Gene (1987) 58:201-216 shows that Saccharomyces cerevisiae is able to perform this recombination.

Each of the foregoing methods requires the isolation of a single clone for production of each targeted protein, a step which is difficult to adapt to high-throughput processing and may result in isolation of mutants rather than intact proteins. Thus none of the foregoing approaches can readily provide large numbers of proteins representing most or all of the entire genome of an infectious agent, the entire proteome of the organism, for example. There remains a need for methods that enable a high throughput protocol for preparing such proteome arrays, which can be analyzed for various interactions and properties.

One of the uses for such arrays is to identify those proteins generated by an infectious organism that are immunoactive as a step toward developing vaccines against that organism. Efforts to identify such antigenic proteins in infectious agents have taken many forms. Proteins have been analyzed in hydrophilicity plots, for example, to ascertain regions that are purported to be exposed and therefore available to the immune system. Alternatively, (as described in U.S. Pat. Nos. 6,620,412 and 6,451,309) 400 monoclonal antibodies were tested for the ability to neutralize virus and then for their ability to protect mice from challenge. Antibodies thus identified were associated with the protein with which they immunoreact. A number of such proteins were identified.

U.S. Application 2003/0082579 describes a method for identifying antigens by screening a protein array derived from an infectious organism with at least one antibody that is present in immune serum elicited by that organism or portions of the organism. The proteins in the array are obtained by PCR amplification of the encoding DNA followed by a second round of PCR amplification to introduce transcription controls; the second round products are then translated into protein in vitro. However, apparently, the method described to obtain the protein array yields inadequate amounts of protein if attempted in a high throughput mode.

These methods thus demonstrate that antigenic proteins useful for vaccine and diagnostic development may be found by screening the proteins of an infectious agent to identify those proteins or portions of proteins that elicit an immune response. However, because they require isolation of a single clone for each protein, they do not provide a high throughput approach for identifying antigens characteristic of an infectious agent that are representative of the full scope of possible antigenic protein or peptide moieties. Such rapid methods are needed in order to quickly respond to develop a vaccine or diagnostic test against a new infectious agent such as, for example, an engineered bioweapon. By permitting synthesis of a protein/peptide array that represents essentially a complete proteome, and by providing means to do so in a practical manner amenable to automation, the present invention offers an opportunity to identify quickly the most promising candidates for diagnostic tests, vaccines and stimulants of T-cell immunity.

### DISCLOSURE OF THE INVENTION

In one aspect, the invention is directed to a method to identify a protein or peptide that has immunogenic activity that can be based on a survey of a substantial proportion of or a substantially complete expression repertoire of the proteins or peptides derived from the genome of an infectious agent such as a virus, protozoan, parasite, or bacterium. The method permits displaying proteins and/or peptides representing 48 to essentially all of the open reading frames in the genome of such an infectious agent and testing each protein and/or peptide in the array with immune serum or plasma from individuals that have been exposed to such infectious agents. Thus ultimately the method makes it possible to identify essentially all of the immunoactive peptides encoded by the genome of an infectious agent.

In general, the invention has a number of aspects, both related to the preparation of peptide/protein arrays useful for the identification of immunoactive peptides or proteins from infectious agents and to the preparation of protein/peptide arrays in general. These methods permit the preparation of arrays which contain peptides or proteins representing significant portions of the genome of an infectious agent. These arrays may be employed to identify immunoactive agents which can elicit cellular and/or humoral responses. The invention also relates to specific antigens so identified and to monoclonal antibodies immunoreactive with them. The antigens, their nucleic acids, and antibodies may all be used to prepare immunologic compositions useful in diagnostic, prophylactic and therapeutic treatment with respect to the infective agents. Thus, in one aspect, the invention relates to methods to obtain expression systems for desired nucleotide sequences which do not employ selection of individual colonies, but rather allow the user to obtain these expression systems from harvested, cultured mixtures of cells. The ratio of nucleic acids to cells used to obtain the transformed cells to be extracted is also an aspect of the invention.

Another aspect of the invention is directed to peptide/protein arrays which either are prepared by the invention method or which represent significant portions of the genome of an infectious organism. The invention also is directed to the antigens thus identified as indicated above and to methods to use these, their corresponding monoclonal antibodies, and nucleic acid molecules encoding them. The antigens that react with antibodies in the serum of infected can be used directly in a serological test to diagnose patients with the infection.

In one aspect, the invention is directed to a method to obtain an expression system for a desired nucleotide sequence. The method may employ host cells transformed with an expression system for the desired nucleotide sequence, or a recombinase-competent host cell transformed with components that can be assembled by such cells into an expression system. The expression system is typically a plasmid; the host cells may be chemically competent bacteria, yeast, or electroporation competent bacteria; in some embodiments the host cells are yeast such as Saccharomyces cerevisiae or bacterium such as E. coli, and may include at least one E. coli strain selected from the group consisting of JC8679, TB1, DH5alpha, DHS, HB101, JM101, JM109, and LE392.

The components of the expression system may include a linearized plasmid, at least one open reading frame from an organism of interest, or a portion of such an ORF, and one or more adapters that are designed to ensure that the ORF can be spliced into the linearized plasmid to create a new plasmid. Thus each such adapter contains a first nucleotide sequence complementary to one end of the linearized plasmid and a second nucleotide sequence that is complementary to one end of the genomic ORF. Two such adapters, properly designed, can be used to insert the ORF into the linearized plasmid, producing a new plasmid having the nucleotide sequence of the ORF inserted in proper reading frame with the plasmid.

The adapters may optionally further include nucleotide sequences coding for one or more added features such as an epitope tag in frame with the ORF, so that the protein expressed will be a fusion protein containing the peptide encoded by the ORF linked to an epitope tag. Such epitope tags may be useful for detection, purification, or localization of the expressed peptide or protein. Epitope tags for this purpose may include, but are not limited to, one or more of the following: a polyhistidine tag encoding 3-12 consecutive histidine residues, commonly 6-10 such residues; a hemagglutinin (HA) tag; a c-Myc tag; a biotin-ligase recognition site; a glutathione-S-adenosyl transferase (GST) tag; a fluorescent protein such as, for example, GFP; a FLAG-tag; and a linker. Since two such adapters are commonly used, these elements may be included on one or both of such adapters; for example, including a poly-his tag on one and an HA tag on the other permits two different detection or localization methods to be employed for a single expressed protein. In some embodiments of the invention, one or more other functional elements are also included on either the adapters or the linearized plasmid; the placement and selection of such elements is well known in the art. Such elements may include promoters, terminator sequences, operons, fusion tags, signal peptides or other functional peptides, antisense sequences, and ribozymes.

The nucleotide sequence to be expressed may include sequence from the genome of an organism, and in some embodiments it is selected to comprise one open reading frame (ORF) from a gene of an organism of interest. In some embodiments the organism is a microorganism, and in some it is an infectious agent. In embodiments where the nucleotide sequence comprises a portion of the genome of an organism such as an infectious agent, adapters employed in the methods herein include one or more epitope tags; representative examples of such tags include HA, c-Myc, and poly-histidine having at least six consecutive his residues.

In one aspect of the invention, both the targeted genomic nucleotide sequence of interest and the linearized plasmid are amplified via PCR before use, and 1-10 ng of the targeted nucleotide sequence and linearized plasmid are used per million cells; in others, the amount of the targeted nucleotide sequence and linearized plasmid may be larger. The molar ratio of nucleotide sequence to plasmid may be about 1:1 in some embodiments; in others it is between 1:10 and 10:1; in still others, it is between 100:1 and 1:100.

The cells are then cultured in the presence of these components and harvested, and the expression system is extracted from a mixture of transformed cells. In another aspect of the invention, isolation of a single clone prior to isolation of the expression system is not required. Rather, the cultured cells are harvested as a “mixture” and the expression system, typically a plasmid, is isolated directly from the harvested cells. The method is thus advantageous for high-throughput and automated means for producing such expression systems and is more successful in recovering plasmids encoding desired proteins or peptides. The latter advantage reflects the ability of the invention method to prevent the loss of the desired expression system through unfortunate selection of a colony that has been mutated or contains an undesired plasmid rather than that sought.

The expression system so produced may be used to produce one or more peptides or proteins in a cellular derived system that can translate the expression system to produce the encoded peptides. The cellular derived system may be inside an intact cell, or it may be a cell-free mixture of the necessary enzymes and components. In some embodiments, the cellular derived system is a bacterium such as Escherichia coli (E. coli); or a yeast; or a prokaryotic cell. In others, it is a eukaryotic cell that may be a mammalian cell such as a reticulocyte or may be an insect cell. In certain embodiments, the expression system is introduced into an antigen presenting cell (APC) such as a dendritic cell, a B cell, or a macrophage. In other embodiments, a translation I transcription system used is a cell-free system, which may be derived from a microorganism such as E. coli, or from a eukaryotic cell such as a reticulocyte, or from a plant cell such as wheat germ.

In one embodiment, the proteins or peptides represent one or more genes of a host genome. Thus the methods of the invention may be used to produce plasmids encoding any subset of the genes of said genome, and may be used to produce a set or array of plasmids encoding most or substantially all of the genes of such a genome. In certain embodiments, the genome is that of an infectious agent.

The expression systems obtained and expressed by the methods of the invention may be used to produce arrays of such proteins or peptides representing the genome of an infectious agent or other organism. These arrays may be used in a further aspect of the invention, which relates to a method to identify an antigen that will generate a humoral and/or cellular immune response. This method comprises exposing at least one protein or peptide produced by the methods herein or exposing an array of proteins and/or peptides representing substantially all of the proteins/peptides encoded by the open reading frames in the genome of an infectious agent to immune serum or plasma or components thereof from a subject that has been exposed to the infectious agent, which subject may be referred to as an “immunized subject.”. Exposure may be, for example, by vaccination using an attenuated form of the infectious agent or portions of the infectious agent or by having been infected by said infectious agent. Proteins/peptides contained in the array which are shown to immunoreact with said serum, plasma or components are identified as promising candidates for vaccine production. If the array includes full-length proteins, the method may further comprise the step of providing an additional array of peptides derived from antigens identified by the foregoing method, wherein such peptides represent segments of the antigenic peptide and allow more precise localization of the antigenic epitope on the protein. Alternatively, full-length proteins or longer peptides may be analyzed using art known methods, such as hydrophilicity plots to identify regions likely to display the greatest immunoactivity. The same proteins or peptides which have been identified as immunologically reactive and of potential utility in vaccine formulations may also be directly useful in serological diagnostic tests to identify the agent responsible for an infected patient's disease. Patients who do not have serum antibodies against the proteins encoded by a given infectious agent, are not infected by the agent. Patients who have antibodies against proteins from the infectious agent were either recently infected or were infected some time in the past.

The peptide/protein arrays used to identify immunoactive peptides or proteins may represent a significant portion of the genome of an infectious agent—e.g., 50%— or they may represent most of (>50%) or substantially all (at least 98%) of the encoded amino acid sequences. In some embodiments, the array of proteins is prepared by the methods of this invention. In some embodiments, the protein or peptide or the array prepared by the methods of the invention is exposed to immune components from a plurality of immunized subjects, and those proteins or peptides that elicit an immune response from at least most of the immunized subjects are identified as immunodominant antigens, and are suitable candidates for inclusion in a vaccine. In some embodiments, they array or protein is also exposed to serum from non-immunized subjects, and the proteins that elicit a response in immunized subjects but not in non-immunized subjects are selected as suitable for use in a vaccine.

A humoral response is detected in some embodiments of the invention by detecting the binding of at least one antibody from an immunized subject to the protein or peptide. Detection of the binding of a protein to an antibody may be observed by methods known in the art, including methods which require the use of a second antibody that is labeled with, for example, a fluorescent label, a radiolabel, or an enzyme.

A cellular immune response may be detected, in some embodiments of the invention. The relevant immune component is a T-cell from an immunized subject. In such embodiments, an immune response is detected by observing the formation of at least one cytokine by a T-cell when said T-cell is contacted with one or more peptides or proteins. For such embodiments, the peptide or protein may be presented by an antigen-presenting cell (APC), and in some embodiments an APC is used to express the peptide or protein from a plasmid obtained by the methods of the invention. In other embodiments, the protein or peptide is expressed as a fusion protein containing at least one epitope tag, and said epitope tag is used to immobilize the protein or peptide onto a surface. In some embodiments, the surface is a particle or bead that is smaller than an APC and can thus be taken up by an APC such as a macrophage; in one such embodiment, the particle is a bead of nickel or a bead that is coated with nickel or with a nickel salt or complex, and the peptide or protein comprises a poly-histidine epitope tag having at least six consecutive histidine residues. The peptide can then be immobilized onto the nickel-comprising head by the affinity of the poly-histidine tag for nickel.

In another aspect, the invention provides a method to detect an immune response of an immune component obtained from a subject to a test material which is contained in a sample with other antigenic materials to which the subject may exhibit an immune response. These circumstances may arise, for example, when the protein to be tested is expressed in a cellular-derived system to which the subject may also have been exposed and to which the subject therefore exhibits an immune response. In this method, the immune component obtained from the subject is first treated with the additional, irrelevant antigenic materials, thereby blocking any immune reaction to the irrelevant antigenic materials, before treating the immune component with said test material. For example, if the protein or peptide to be tested is produced in a system derived from E. coli, immune component samples derived from human subjects may be treated with E. coli extracts in order to block the background immune response which humans appear to exhibit to various E. coli antigens. Lysates or extracts of E. coli would then be used preliminarily to treat the sample from the subject.

To summarize, the invention is directed to a method to provide individual proteins or peptides encoded by an open reading frame (ORF) or a portion thereof which comprises effecting expression of an insert encoding said protein or peptide in an expression system, (e.g., plasmids) which have been extracted from mixtures (not clones) of recombinase competent cells that have been modified to contain said insert and a linearized plasmid; wherein said linearized plasmid and said insert have been ligated by homologous recombination in vivo in said cells and wherein said insert has been amplified from said ORF or a portion thereof. In one particular embodiment, the linearized plasmid has itself been amplified. The amplification can be by PCR. Expression to produce protein may, for example, be in a cell-free system, or in cells that provide desirable post-translation modification. The method can allow a multiplicity of proteins or peptides to be generated simultaneously. In some embodiments, 10, 50, 100, 200, 400, 600, 800, 1000, 1500, 2000, or more than 2000 different proteins or peptides can be generated simultaneously.

The invention provides a method to produce samples of most or substantially all of the proteins or peptides encoded by the genome of an infectious agent or organism. The proteins or peptides thus obtained may be separately contained, or they may be spotted onto a substrate such as nitrocellulose or onto a plate or chip to produce an array of proteins or peptides on a test surface. In some embodiments, each of these proteins or peptides may be fused to one or more epitope tags, which permit detection, localization or purification of the protein after it is translated. The epitope tags may be used to immobilize the protein or peptide on a surface bearing or consisting of a complementary binding material such as, for example, a nickel surface that is capable of binding tightly to a poly-histidine tag of an expressed protein. Thus, in some embodiments, the peptide of interest is expressed fused to an epitope tag, and said epitope tag is used to immobilize the peptide onto a surface such as a bead or a well of an assay plate. In one such embodiment, the epitope tag is a poly-histidine sequence containing at least six consecutive histidine residues, and the surface onto which one or more of such proteins is immobilized comprises nickel.

In still another embodiment, the invention is directed to a method to obtain plasmids which contain inserts comprising a nucleotide sequence that is an ORF or portion thereof, which comprises extracting said plasmids from a mixture (not clones) of recombinase competent microorganisms that have been modified to contain a linearized vector and an amplified nucleic acid comprising said ORF or portion thereof and have effected recombination of said insert and said linearized plasmid through homologous recombination.

In still another aspect, the invention is directed to a method to identify antigens that will generate a humoral response to an infectious agent, which method comprises contacting an array of proteins and/or peptides obtained by the method of the invention with immune serum or plasma or immunoglobulins contained therein, each of which is obtained from a subject exposed to the infectious agent optionally in an attenuated form, or to some portion thereof, in a manner calculated to elicit an immune response, and identifying as a suitable antigen those proteins or peptides which immunoreact with the plasma, serum, or separated immunoglobulins. In some embodiments, the peptides/proteins represent most of or substantially all of the genome of said infectious agent, and the immunoreactivity includes binding to at least one antibody produced by the subject in response to the infectious agent. The proteins or peptides may be derived according to the methods described above using in vivo recombination to obtain plasmids which are then subjected to expression in a cellular derived system, which may be inside intact cells or may be a cell-free system. It may in some cases be desirable to treat the serum or plasma with a lysate of the organism furnishing the cellular derived system used to express the protein in order to minimize background immunoreactivity. In some embodiments, the cellular derived system is obtained from E. coli, and an extract or lysate of E. coli is used to block background immune responses to the components of the cellular derived system. Binding of the protein or peptide to an antibody may be detected in some embodiments by use of a secondary antibody that is labeled for ease of detection with a fluorescent, radioactive, or enzymatic labeling group.

In other aspects, the invention is directed to a method to identify antigens that generate cellular responses to an infectious agent. This process may be similar to that set forth above, but may employ dendritic cells or other cellular components of the immune system of a subject as the diagnostic agent for immunoactivity. In certain embodiments, the proteins or peptides provided by the methods described above are immobilized on a substrate such as a bead, as for example by incorporating a poly-histidine epitope tag on the expressed protein which allows that protein to be immobilized on a nickel-coated bead, and the immobilized protein or peptide is then exposed to an APC. Advantageously, the substrate is a structure such as a bead that is smaller than an APC and is thus subject to internalization by such APC. Said APC is then exposed to at least one type of responder cell such as a T-cell from a subject immunized against the infectious agent by the methods discussed above, and the production of one or more cytokines by said responder cells or T-cells demonstrates the presence of an immune response to that protein. Thus in this embodiment, the immune response may be detected by detecting the formation of one or more cytokines when the T-cells are exposed to an APC which has been exposed to the peptide or protein. Alternatively, the immune response may be detected by observing proliferation of cytotoxic activity of said responder cells or T-cells.

Once an antigenic protein has been identified, the methods of the invention may also be used to scan the protein in to identify more precisely the region on the protein that is immunogenic. This is done by providing primers designed to express segments of the protein that may be 10 to 20, or 20 to 30, or 20-50, or 20-100 amino acids in length, for example, though shorter or longer segments may be used as appropriate. These shorter peptides are then expressed and analyzed by the methods of the invention, and those peptides that give rise to antigenic effects are thus identified. Optionally, these segments may be designed to overlap in order to minimize the chance that an antigen will be missed because it is split between two segments.

In other aspects, the invention is directed to arrays of proteins/peptides obtained by the invention method, to antigens identified from said arrays, to immunodominant antigens identified by the methods of the invention, and to vaccine compositions containing at least one of such antigens as well as DNA vaccine compositions containing nucleotide sequences that encode at least one of such antigens and to serological diagnostic tests containing at least one of the antigens identified by the above methods.. In other aspects, it is directed to antibodies and especially monoclonal antibodies specific for at least one of said antigens and to compositions containing such antibodies. Still further aspects are directed to methods to immunize a subject with the compositions of the invention, including antigens, antibodies, vaccines and DNA vaccines, and methods to use the nucleic acids and/or antigens identified by these methods therapeutically or diagnostically, such as to unambiguously determine whether a person is or was previously infected with a particular organism.

In certain embodiments of the invention, the methods described herein for production of expression systems are applied to incorporate each gene of a set selected from the genome of an organism into its own plasmid, optionally including epitope tags; and an array of such proteins is produced, representing most or substantially all of the proteins (the entire proteome) of that organism. The organism may be an infectious agent such as Bacillus anthracia (anthrax), Clostridium botulinum, Yersinia pestis, Variola major (smallpox) and other pox viruses, Francisella tularensis (tularemia) or Viral hemorrhagic fevers including Arenaviruses (e.g., LCM, Junin virus, Machupo virus, Guanarito virus, Lassa Fever), Bunyaviruses (e.g., Hantaviruses, Rift Valley Fever), Flaviruses (e.g., Dengue) or Filoviruses (e.g., Ebola, Marburg). The organism may also an infections agent such as Burkholderia pseudomallei, Coxiella burnetii (Q fever), Brucella species (brucellosis), Burkholderia mallei (glanders), Ricin toxin (from Ricinus communis), Epsilon toxin of Clostridium perfringens, Staphylococcus enterotoxin B, Typhus fever (Rickettsia prowazekii) or Food and Waterborne Pathogens including bacteria (e.g., Diarrheagenic E. coli, Pathogenic Vibrios, Shigella species, Salmonella, Listeria monocytogenes, Campylobacter jejuni, Yersinia enterocolitica), viruses (Caliciviruses, Hepatitis A), or protozoa (e.g., Cryptosporidium parvum, Cyclospora cayatanensis, Giardia Iamblia, Entamoeba histolytica, Toxoplasma, Microsporidia). The organism may also be an infectious agent such as viral encephalitides including West Nile Virus, LaCrosse, California encephalitis, VEE, EEE, WEE, Japanese Encephalitis Virus or Kyasanur Forest Virus. The organism may also be an infectious agent such as Nipah virus, hantaviruses, Tickbome hemon-hagic fever viruses (e.g., Crimean-Congo Hemon-hagic fever virus), Tickbome encephalitis viruses, Yellow fever, Multi-drug resistant TB, Influenza, Rickettsias, Rabies or Severe acute respiratory syndrome-associated coronavirus (SARS-CoV). In some embodiments it is Francisella tularensis, human papillomavirus, West Nile virus, Burkholderia pseudomallei, or Plasmodium falciparum, Mycobacterium tuberculosis or vaccinia. The proteins so produced may be fonnatted into an array, as by spotting each protein or peptide produced onto a test surface such as a chip. Proteins may be localized into such arrays by non-specific binding of the protein to the test surface, as to nitrocellulose, or by specific association of an epitope tag if present on the protein or peptide to a feature of the surface that binds that epitope tag; for example, if the protein or peptide comprises a poly-histidine tag, a nickel-containing surface may be used.

The an-ay may contain a selected set of the proteins of such organism, or it may include proteins and/or peptides representing at least about 50%, 60%, 70%, 80%, 90%, 95%, or 98% or more, i.e., substantially all of the genome of the infectious agent. The number of such proteins and/or peptides will be at least 100, 200, 300, 400, 500, 1000, 1500, 2000, or more than 2000 different sequences. In such embodiments, the array may be obtained by preparing several separate arrays that collectively represent such fractions of the organism's proteome. Thus in some embodiments, the invention provides a method to produce an array of proteins on a test surface, where the array represents selected portions of the proteome of an infectious agent, up to and including essentially the entire proteome. Such proteomic arrays may be used to determine the strain of a pathogenic organism that has infected a subject, as well as for the identification of immunodominant antigenic proteins, or for determination of any other activity or property the proteins may possess. In still other aspects, the invention is directed to monoclonal antibodies immunoreactive with the identified antigens and methods to confer passive immunity using such antibodies.

### BRIEF DESCRIPTION OF THE DRAWINGS

FIG. 1 shows a diagram of the host vector, and the nucleotide sequence surrounding the BamH1 site. As shown, the in-frame insertion of the PCR-amplified fragment from the genome occurs after the glutamate codon GAG at base number 206. The 5’ homologous cloning region starts at base number 206 and extends 33 bases upstream and results in an in-frame fusion with a 10× histidine tag. The 3′ homologous cloning region starts at base number 212 and extends 33 bases downstream resulting with the HA tag and terminating with aTAA stop codon.

FIG. 2 shows gels displaying a set of cleaned PCR products from vaccinia and Francisella tularensis.

FIG. 3 shows gels of phenol-chloroform lysed cells to give total nucleic acids from overnight cultures of the E. coli effecting recombination.

FIG. 4 shows plasmids from minipreps of selected colonies from the overnight cultures used in FIG. 3.

FIG. 5 shows SDS PAGE gels run on translated products of the plasmid minipreps of FIG. 4 said gels being probed with anti-polyhistidine antibody.

FIG. 6 shows dot-blots of the translations of the plasmids ofFigure 4 probed with anti-histidine antibody or anti-HA antibody.

FIGS. 7A-7D show exemplary results of SDS PAGE of immunoreactive proteins identified on dot-blots probed with anti-histidine tag (FIG. 7A) anti-HA tag (FIG. 7B) with VIG without E. coli lysate (FIG. 7C) and with vaccinia immune globulin (VIG) in the presence oflysate (FIG. 7D).

FIG. 8 shows quantitative results of a dot-blot of individual vaccinia proteins with and without treatment of the VIG with E. coli lysate.

FIG. 9 shows a microarray of vaccinia proteins identifying DBL, F13L, H3L, H5R, A56R and 644 as immunoreactive with VIG.

FIG. 10 shows total nucleic acids obtained from the transformation mixtures which include the inserts from vaccinia described above.

FIG. 11 shows SDS PAGE results of the translation reactions performed on the plasmids obtained from mixtures of cells, probed with anti-polyhistidine.

FIGS. 12A-12D show dot-blots for proteins of FIG. 11 applied without purification to nitrocellulose to provide an array of vaccinia proteins. FIGS. 12A-D show the results when the dot-blots are proved with anti-histidine, anti-HA, VIG without lysate, and VIG with lysate, respectively. FIG. 12E illustrates a protein array feature map that identifies the proteins that are spotted on the corresponding location on the arrays in FIGS. 12A-D; notice that the feature map of FIG. 12E has 11 rows and 12 columns of protein IDs, corresponding to the 11 rows and 12 columns of spots on the arrays in FIG. 12 A-D.

FIG. 13 shows a smaller protein array showing the results with and without E. coli lysate.

FIGS. 14A and 14B show the results of vaccinia dot-blots with respect to naive and vaccinia virus-immunized mouse and human sera.

FIGS. 15A-15C show a scan of the H3L envelope protein of vaccinia, where the protein sequence was divided into 10 segments, each overlapping its neighbor or neighbors by 20 amino acids, as described in Example 8.

### MODES OF CARRYING OUT THE INVENTION

One embodiment of the invention provides a high throughput method to obtain an array of proteins and/or peptides representative of those encoded in the genome of an infectious agent so that the arrays can be tested for their ability to effect a humoral and/or cellular immune response. The method for preparing the proteins in the array is applicable to the preparation of proteins in general, from any source. In particular, the high throughput advantages inherent in the method are applicable in providing a repertoire of proteins and peptides from infectious agents. The method could also be used for providing a multiplicity of proteins and/or peptides encoded by any nucleic acid of known sequence so that individual

amplified portions or inserts may be provided to plasmids replicable in recombinase-containing microorganisms. The invention method for preparation of such proteins differs from those employed previously in that it employs DNA extracted from mixtures of microorganisms obtained by culturing the components of a transformation mixture rather than isolating individual clones. This is advantageous as isolation of clones often results in obtention of a mutant rather than the desired native form of the protein. Further, the invention method may employ, in the screening phase, unpurified forms of the proteins encoded by and expressed from vectors obtained from these mixtures. As a result, the present method greatly simplifies automation of the overall process and adoption of high-throughput processing.

Using the method of the invention, it has been possible to identify particular proteins from vaccinia that will be potent vaccines. This is of considerable significance as the use of attenuated virus is sometimes associated with unwanted side effects. It would be preferable to utilize a single protein or defined mixture of proteins, rather than the complex infectious agent in attenuated form. This is done currently, for example, using hepatitis B surface antigen.

The invention method is applicable, as stated above, to nucleic acids that encode a multiplicity of proteins and peptides in general where the relevant nucleotide sequence is known, so that appropriate primers can be employed to effect the amplification of the desired insert. As described in, for example, US2003/0082579 and US2003/0044820, both incorporated herein by reference, the designed primers may include adapter sequences that provide for the desired homologous recombination with a linearized vector. The extended primers themselves and/or the linearized vector may then provide appropriate control sequences, such as promoters and terminators to effect expression as well as “tags” such as histidine tags, FLAG tags, and the like, to permit strengthened binding to an appropriate solid surface or, if desired, purification of the expressed protein. Commonly, the linearized vector is also amplified by PCR, rather than using the more traditional method of vector digestion, which can result in vectors which fail to contain inserts.

In the overall method of the invention, a nucleic acid molecule, such as an infectious agent genome, that encodes a multiplicity of proteins or peptides and whose nucleotide sequence is known, is used as the substrate. Each segment that encodes a protein or peptide of interest is individually (i.e., in an individual reaction mixture) amplified using PCR or other amplification techniques employing primers that contain both a sequence complementary to an end portion of the coding sequence and an adapter that may encode a tag and/or a sequence that controls expression, but which, in any event, is homologous to sequences provided on a linearized plasmid. The individually amplified segment and linearized plasmid are then cotransfected into a recombinase-containing microorganism to permit recombination in vivo. The recombinase-containing organisms may be, for example, yeast or may be a chemically competent E. coli (or, less desirably, an electroporation competent E. coli). Suitable chemically competent E. coli include the strains JC8679, TB1, DH5a, HB101, JM101, JM109 and LE392. Saccharomyces are particularly effective with regard to recombinase-containing yeast.

The ratio of DNA to cells in the transfection reaction may be as high as 100 ng/million cells; however, ratios of as low as 1-10 ng, 5-10 ng, 1-5 ng or 1-3 ng/million cells may also be used. It is often desirable to provide the linearized plasmid and the desired nucleotide sequence in about a 1:1 molar ratio, though ratios from 5:1 to 10:1 to 100:1 may be used, and ratios of 1:5 to 1:10 to 1:100 may also be used.

The cells thus treated with the amplified insert and the amplified linearized vector are cultured on suitable medium, often overnight. The resultant is a mixture of cells, most of which will contain the desired recombined vector having the anlplified segment of the desired nucleotide sequence inserted in the correct orientation. (Directionality is ensured by the design of the primers to match the homologous portions of the linearized plasmid.) Rather than isolating individual colonies, which risks loss of the desired insert in favor of, for example, a mutant, the cells are harvested from the culture and extracted directly to obtain the plasmid DNA. The plasmid mixture thus obtained is then subjected to transcription/translation either by transfecting the DNA into suitable host cells, or commonly for the purposes of high throughput, in an in vitro translation system. Such in vitro translation systems are commercially available, and methods for their use are well known to those of skill in the art. The resulting protein or peptide can then be directly spotted onto a solid support, which support may be a portion of an array of proteins and peptides prepared on any suitable surface, such as the wells of a microtitre plate or segmented nitrocellulose. The protein may, if desired, be purified by methods known in the art, or by using a tag that was encoded into it from the primer or plasmid, or, alternatively, the transcription/translation mixture can be used directly without further purification of the protein to provide the protein or peptide to the solid support. Purified or substantially purified proteins produced by this method are one aspect of the invention. Those proteins or peptides may be naturally occurring peptides or modified versions comprising one or more additions such as an epitope tag as further described herein. Where the proteins are adhered to a support, the solid support may, itself, be supplied with a counterpart ligand to a tag on the protein or peptide.

In order to obtain an array of proteins, the foregoing sequence of steps is performed with respect to as many ORF's or portions thereof as desired. It may be advantageous to obtain only a relatively small number of proteins or peptides as members of the protein/peptide array if promising candidates are already known for whatever screen is to be performed on the array. However, a multiplicity of nucleotide sequences may be turned into proteins or peptides; as many as 50, 100, 500, 1,000 or more. If the genome of an infectious agent is used, for example, or the genome of any prokaryote, the array may include at least 10%, 20%, 50%, 75%, 90%, 95% or 100% of the proteins and peptides expressed. The resultant array may represent substantially the entire proteome of the organism, i.e. at least about 98% of the proteome or only a portion thereof, or may represent individual peptide portions of the proteins in the proteome, or a combination of full-length proteins and partial sequences.

In order to facilitate the preparation of an array of peptides or proteins, it may be advantageous to fuse the peptide or protein of interest with a short peptide tag, which is commonly 6 to 20 amino acids in length, that binds to a specific functional group. Such binding tags can then be used for purification of the protein or to affix the protein to a test surface, or to detect the presence of the protein. Such binding tags consisting of short sequences of amino acids are well known and are commonly referred to as epitope tags. For example, a hemagglutinin (HA) epitope tag (such as the human influenza hemagglutinin protein, YPYDVPDYA) or a c-Myc epitope tag (a 10 amino acid segment of the human protooncogene myc, EQKLISEEDL) may be fused to the peptide or protein to be expressed by incorporating the appropriate nucleotide sequence into the adapter used to insert the genomic nucleic acid into an expression plasmid. Antibodies to the c-Myc, HA, or other epitope tag may then be used to detect or localize the expressed peptide.

Similarly, a poly-histidine tag may serve as an epitope tag and may be incorporated into the expressed protein by proper design of the adapters used to insert the genomic nucleic acid into the vector used for expressing the protein. A poly-histidine epitope tag may contain 3 to 12 consecutive histidine residues, commonly 6-10 consecutive histidine residues. Such poly-histidine tag will specifically and tightly bind to a nickel surface; thus the expressed peptide or protein containing such a tag will bind tightly to a nickel bead, a nickel-coated surface, or an affinity column comprising nickel or a nickel salt or complex such as, for example, nickel nitrilotriacetic acid (Ni-NTA). An array of proteins or peptides containing poly-histidine tags can thus be produced in a 96-well format by coating each well with nickel or a nickel salt or complex, then placing a solution of each protein or peptide into such a nickel-coated well and allowing the protein to become affixed to the surface. Similarly, such proteins can be attached to a bead for convenient display by making beads of nickel or by plating beads of other material with nickel or a nickel salt or complex. In one embodiment, the proteins of a genome are tagged with a poly-his tag comprising at least 6 consecutive histidine residues and are allowed to adhere to 1 um nickel beads; these beads are then used to assay for immunological response by T-cells as described in Example 9, infra.

Where desired, it is also possible to attach two different tags: a nucleotide sequence coding for a first tag can be included near the 5′ end of the nucleic acid inserted into the plasmid to attach a tag at the N-terminal of the expressed protein, and a nucleotide sequence coding for a second tag can be included near the 3′ end of the nucleic acid inserted into the plasmid to attach a tag near the C-terminal end of the expressed protein. These tags could be the same, to insure recognition in case one terminus is buried and thus inaccessible; or they may be different, to enable two different capture or detection methods to be used. Other tags useful for detection, localization or purification may also be attached to the genomic protein as needed. Such tags include glutathione-S-transferase (GST), biotinylation signals, green fluorescent protein (GFP) and the like, each of which can be incorporated by methods well known in the art.

Once the desired peptides/proteins or array of peptides/proteins is obtained, it may be screened for any desired property or reactivity. One example of such use is screening for immunoactive peptides and proteins. The immunoactivity may be with respect to the humoral or the cellular system. In either case, a screening agent obtained from a subject that has been exposed to the infectious agent or some portions thereof is required. Optionally, the array of proteins or peptides may be screened against one or more immune components (serum, sputum, plasma, T-cells, etc.) from multiple subjects, each of which has been exposed to the infectious agent or some portion of it such as its envelope proteins or lysed cells, or one or more of its proteins. This permits determination of which antigens elicit immune responses in multiple subjects: those most commonly recognized are referred to as immunodominant antigens. A family of antigens may be useful in a serological diagnostic test or in a vaccine comprising several of these immunodominant antigens.

The methods of the invention can be applied to a variety of genomes, and are often usefully applied to the genomes of infectious agents, including viruses, fungi, bacteria, protozoa and the like as well as multicellular parasites such as flatworms, flukes, roundworms, and the like. By providing methods to quickly produce an array of proteins that represent most of all of the proteome of such an infectious agent, the invention makes it possible to quickly identify those genes and proteins most useful for the development of vaccines or diagnostic tests against a particular infectious agent.

Thus, as used herein, the term “immunoactive” refers to the ability of a protein or peptide to elicit an immune response, whether that response is humoral or cellular, or both. A humoral immune response is an adaptive protection mechanism that is characterized by the production of antibodies, while a cellular immune response is characterized by the production and/or activation of cells such as activated natural killer (NK) cells and cytotoxic T-lymphocytes (T-cells, or CTL). Similarly, “antigen” refers to such immunoactive proteins or peptides, regardless of the nature of the immune response elicited. “Immunodominant antigen” refers to an antigen that elicits an immune response in most or all subjects exposed to the antigen; such immunodominant antigens are most likely to provide effective vaccine components or elicitors of antibody production for use in passive immunization methods, and are therefore often especially useful as components of an immunologic composition and will also be useful in serological diagnostic tests..

T cells recognize peptide/MEC complexes on the surface of other cells. Such cells are often referred to as antigen presenting cells (APCs). Although effector cells can mediate their functions by recognizing such complexes on virtually any cell type, naive cells are most efficiently activated by a set of specialized APCs, the dendritic cells (DCs).

“Array” as used herein refers to a collection of materials systematically positioned on at least one test surface, including materials contained in wells or depressions formed on said surface, where the placement of the material is correlated to the identity of the material. An array generally contains at least about 10 materials so positioned, and often contains at least 100 or 200 or 500, or it may contain 1000 or more materials. It includes materials spotted onto a chip, plate, or nitrocellulose substrate, for example, and materials contained in the wells of 96-well and 384-well and similar plates, as long as the materials are retained in the location where they were placed, whether they are retained due to physical or chemical forces. An array may comprise multiple plates, chips or other surfaces. A microarray is a miniaturized array that may be designed to minimize reagent volumes, for example. While the arrays described herein are often arrays of antigenic peptides, the invention also includes arrays of antibodies that are selective for such antigenic peptides.

The antigens identified by the method of the invention may be peptides or proteins and are used to prepare immunologic compositions for protecting subjects against infection by the infectious agent or to generate monoclonal antibodies useful for providing passive immunization or for purification or detection of the antigens. Such immunologic compositions may be vaccines that induce a subject to produce an immune response such as the production of antibodies, or they may themselves be antibodies or active immunological materials that provide passive immunity. Anti-idiotypic antibodies or nucleic acids that generate them may be used in lieu of the antigens themselves. They may also be nucleic acid vaccines that generate one or more antigenic epitopes, wherein the nucleic acid can be taken up by the subject's own cells. They may be accompanied by functional elements such as promoters that effect production of the encoded antigenic protein or peptide, or may be naked DNA.

The invention also includes those peptides and antigens that are substantially homologous to those identified by the methods of the present invention, as well as immunologic compositions derived from such substantially homologous antigens. Thus it includes diagnostic tests or vaccines containing peptides or proteins that are substantially homologous to those peptides or proteins identified by the methods described herein; it includes antibodies specific for antigens that are substantially homologous to those antigens identified by the methods described herein; and it includes nucleic acids having nucleotide sequences encoding these substantially homologous peptides or proteins.

The term “substantially homologous”, when used herein with respect to a protein or peptide, means a protein or peptide corresponding to a reference protein or peptide, wherein the protein or peptide has substantially the same structure and function as the reference, for example, where only changes in amino acids sequence not affecting function occur. Thus, in the present application, the substantially homologous peptides and proteins are immunoactive and have similar structures to the reference. With regard to structure, the percentage of identity between the substantially homologous versus the reference protein or peptide is at least 65%, or at least 75%, or at least 85%, or at least 90%, or at least 95%, or at least 99%.

Alignment of protein sequences for identity comparison can be conducted by art known method. Useful methods for comparison of protein sequences include the local homology algorithm of Smith & Waterman, Adv. Appl. Math. 2: 482 (1981); the homology alignment algorithm of Needleman & Wunsch, J. Mol. Biol. 48: 443 (1970); the search for similarity method of Pearson & Lipman, Proc. Nat'l. Acad. Sci. USA 85: 2444 (1988); computerized implementations of these algorithms (GAP, BESTFIT, PASTA, and TFASTA in the Wisconsin Genetics Software Package, Genetics Computer Group, 575 Science Dr., Madison, Wis.); and visual inspection (see generally, Ausubel et al., infra).

An example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information at the web site www.ncbi.nlm.nih.gov. This algorithm involves first identifying high scoring sequence pairs (HSPs) by identifying short words of length Win the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. For amino acid sequences, the BLASTP program uses as defaults a wordlength (W) of 3, an expectation (E) of 10, and the BLOSUM62 scoring matrix (see Henikoff & Henikoff, Proc. Natl. Aca. Sci. USA (1989) 89: 10915).

Sequence alignments may also be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method may be, for example, KTUPLE 1, GAP PENALTY=3, WINDOW=5 and DIAGONALS SAVED=5.

In the alternative, proteins or peptides are also considered substantially homologous herein when they are immunologically cross reactive. A variety of immunoassay formats may be used to select antibodies specifically immunoreactive with a particular protein or peptide. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

One of ordinary skill in the art will recognize that individual substitutions, deletions or additions that alter, add or delete a single amino acid or a small percentage of amino acids (for example, less than about 5%, or for example, less than about 1%) in a sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also, Creighton (1984) Proteins, W.H. Freeman and Company. Conservatively modified variations of a described nucleic acid nucleotide sequence or polypeptide amino acid sequence is implicit in each described sequence.

One aspect of the present invention relates to nucleotide sequences that encode all or a substantial portion of the amino acid sequence encoding the proteins or substantial portions thereof identified herein. (One example of such proteins is H3L Western Reserve Strain, H3L Copenhagen Strain and H3L Variola Major Bangladesh Strain proteins.) A “substantial portion” of a protein comprises enough of the amino acid sequence to afford putative identification, either by manual evaluation of the sequence by one skilled in the art, or by computer-automated sequence comparison and identification using algorithms such as BLAST (Basic Local Alignment Search Tool; Altschul, S. F., et al., (1993) J. Mol. Bioi. 215:403-410). In general, a sequence of nine or more contiguous amino acids is necessary in order to putatively identify a protein as homologous to a known protein. Substantially homologous protein fragments may be identified by the percent identity of the amino acid sequences of the fragments compared to those proteins disclosed herein.

As noted in greater detail below, the immunogenic peptides can be prepared synthetically, such as by chemical synthesis or by recombinant DNA technology, or isolated from natural sources such as whole viruses or other infectious agents. Although the peptide will often be substantially free of other naturally occurring host cell proteins and fragments thereof, in some embodiments the peptides can be synthetically conjugated to native fragments” or particles.

Peptides having the desired activity may be modified as necessary to provide certain desired attributes, e.g., improved pharmacological characteristics, while increasing or at least retaining substantially all of the antigenic activity of the unmodified peptide. For instance, the peptides may be subject to various changes, such as substitutions, either conservative or non-conservative, where such changes might provide for certain advantages in their use, such as improved MHC binding. The range of amino acid substitutions may also include using D-amino acids. Such modifications may be made using well known peptide synthesis procedures, as described in e.g., Merrifield, Science 232:341-347 (1986), Barany and Merrifield, The Peptides, Gross and Meienhofer, eds. (N.Y., Academic Press), pp. 1-284 (1979); and Stewart and Young, Solid Phase Peptide Synthesis, (Rockford, Ill., Pierce), 2d Ed. (1984), each of which is incorporated herein by reference.

The pharmaceutical compositions for therapeutic treatment are intended for parenteral, topical, oral or local administration. In some embodiments it may be desirable to include in the pharmaceutical compositions of the invention at least one component which primes CTL. Lipids have been identified as agents capable of priming CTL in vivo against viral antigens. For example, palmitic acid residues can be attached to the alpha and epsilon amino groups of a Lys residue and then linked, e.g., via one or more linking residues such as Gly, Gly-Gly-, Ser, Ser-Ser, or the like, to an immunogenic peptide. The lipidated peptide can then be injected directly in a micellar form, incorporated into a liposome or emulsified in an adjuvant, e.g., incomplete Freund's adjuvant. In one embodiment a particularly effective immunogen comprises palmitic acid attached to alpha and epsilon amino groups of Lys, which is attached via linkage, e.g., Ser-Ser, to the amino terminus of the immunogenic peptide.

The peptides of the invention can be prepared in a wide variety of ways. Because of their relatively short size, some such peptides (discrete epitopes or polyepitopic peptides) can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984), which is incorporated herein by reference.

The peptides of the present invention and pharmaceutical and vaccine compositions thereof are useful for administration to mammals, particularly humans, to therapeutically treat and/or prevent infections. For pharmaceutical compositions, the immunogenic peptides of the invention are often administered to an individual already infected with the infectious agent of interest. Those in the incubation phase or the acute phase of infection can be treated with the immunogenic peptides separately or in conjunction with other treatments, as appropriate. In therapeutic applications, compositions are administered to a patient in an amount sufficient to elicit an effective CTL response to the infectious agent's antigen and to cure or at least partially arrest symptoms and/or complications. An amount adequate to accomplish this is defined as a “therapeutically effective dose” or “unit dose”. Amounts effective for this use will depend on, e.g., the peptide composition, the manner of administration, the stage and severity of the disease being treated, the weight and general state of health of the patient, and the judgment of the prescribing physician. Generally for humans the dose range for the initial immunization (that is for therapeutic or prophylactic administration) is from about 1.0 μg to about 20,000 μg of peptide for a 70 kg patient, typically about 50 μg, 100 μg, 150 μg, 200 μg, 250 μg, 300 μg, 400 μg, or 500 μg, 1000 μg, 2000 μg, 5,000 μg, 10, 000 μg, 15,000 μg, or 20,000 μg, sometimes followed by boosting dosages in the same or dose range, though not necessarily the same actual dose, pursuant to a boosting regimen over weeks to months depending upon the patient's response and condition by measuring specific CTL activity in the patient's blood.

The identification of patients for treatment with such vaccine compositions and of population segments for prophylactic administration of such vaccine compositions is well within the skill of one of ordinary skill in the art. For therapeutic use, administration should begin at the first sign of infection or shortly after diagnosis in the case of acute infection. This is followed by boosting doses until at least symptoms are substantially abated and for a period thereafter. In chronic infection, loading doses followed by boosting doses may be required.

The peptide compositions can also be used for the treatment of chronic infection and to stimulate the immune system to eliminate, e.g., virus-infected cells in carriers. It is often important to provide an amount of immuno-potentiating peptide in a formulation and mode of administration sufficient to effectively stimulate a cytotoxic T-cell response. Thus, for treatment of chronic infection, immunizing doses followed by boosting doses at established intervals, e.g., from one to four weeks, may be required, possibly for a prolonged period of time, to effectively immunize an individual.

Frequently it is desirable to prepare a cocktail containing at least two, or at least three, or five or more antigens from an infectious agent to ensure that the vaccine is effective for a broad range of recipients. In addition to the primary antigenic activity of a peptide, it is sometimes also useful to determine if non-immunized subjects also exhibit an immune response to the peptide. A cocktail of immunogenic peptides to be used as a vaccine is sometimes selected to include at least 2 or at least 3 proteins that react with serum from immunized subjects and do not react with serum from non-immunized subjects.

Delivery of the compositions of the invention can be by any methods familiar to those of skill in the art, including oral, inhalation, topical, and injection methods. Frequently, the pharmaceutical compositions are administered parenterally, e.g., intravenously, subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The compositions of the invention may also be administered via liposomes. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes either filled or decorated with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369. Other types of adjuvants and emulsions can also be used such as SAF-1, PROVAX and Tomatine. Also alum can be used to help stimulate the immune response against the formulated protein or peptide antigens.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 0.01-95% of active ingredient, that is, one or more peptides of the invention, and more preferably at a concentration of 0.1% to 75%, or 0.2%-50% or 1%-20%.

For aerosol administration, the immunogenic peptides are generally supplied in finely divided form along with a surfactant and propellant. Typical percentages of peptides are 0.01%-20% by weight, or 1%-10%. The surfactant must, of course, be nontoxic, and is generally soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, commonly 0.25-5%. The balance of the composition is ordinarily propellant. A carrier can also be included, as desired, as with, e.g., lecithin for intranasal delivery.

The peptides of the invention can also be expressed by attenuated viral hosts, such as vaccinia or fowlpox. This approach involves the use of vaccinia virus as a vector to express nucleotide sequences that encode the peptides of the invention. Vaccinia vectors and methods useful in immunization protocols are described in, e.g., U.S. Pat. No. 4,722,848. Another vector is BCG (Bacille Calmette Guerin). BCG vectors are described, e.g., in Stover, et al. (Nature 351:456-460 (1991)), which is incorporated herein by reference. A wide variety of other vectors useful for therapeutic administration or immunization of the peptides of the invention, e.g., Salmonella typhi vectors and the like, will be apparent to those skilled in the art from the description herein.

For therapeutic or immunization purposes, peptides of the invention can be administered in the form of nucleic acids encoding one or more of the peptides of the invention. The nucleic acids can encode a peptide of the invention and optionally one or more additional molecules. A number of methods are conveniently used to deliver nucleic acids to a patient. For instance, nucleic acid can be delivered directly, as “naked DNA”. This approach is described, for instance, in Wolff, et al., Science 247: 1465-1468 (1990) as well as U.S. Patent Nos. 5,580,859 and 5,589,466, each of which is incorporated herein by reference. Nucleic acids can also be administered using ballistic delivery as described, for instance, in U.S. Pat. No. 5,204,253. Particles comprised solely of DNA can be administered. Alternatively, DNA can be adhered to particles, such as gold particles. As with delivery of peptides, it is frequently desirable to prepare a cocktail containing at least two, or at least three, or five or more nucleic acids encoding antigenic peptides from an infectious species to ensure that the DNA vaccine is effective for a broad range of recipients.

The nucleic acids can also be delivered complexed to cationic compounds, such as cationic lipids. Lipid-mediated gene delivery methods are described, for instance, in WO96/18372; WO 93/24640; Mannino & Gould-Fogerite, BioTechniques 6(7): 682-691 (1988); Rose U.S. Pat No. 5,279,833; WO 91/06309; and Feigner, et al., Proc. Natl. Acad. Sci. USA 84: 7413-7414 (1987), each of which is incorporated herein by reference.

Purified-plasmid DNA can be prepared for injection using a variety of formulations. The simplest of these is reconstitution of lyophilized DNA in sterile phosphate-buffer saline (PBS). A variety of methods have been described, and new techniques may become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

The immunologic compositions will contain effective amounts of one or more of the identified antigens along with suitable excipients. Vaccines for injection will typically contain excipients and additional ingredients to confer stability. The nature of the composition will depend on the route of administration which may be, for example, intravenous, intramuscular, subcutaneous, or intraperitoneal injection, or may be transmucosal, transdermal, or oral. The design of compositions for vaccines is well established, and is described, for example, in Remington's Pharmaceutical Sciences, latest edition, Mack Publishing Co., Easton, Pa., and in Plotkin and Orenstein's book entitled Vaccines, 4th Ed., Saunders, Philadelphia, PA (2004), each of which is incorporated herein by reference.

Immunizations with individual proteins, as opposed to inactivated viral particles, may require adjuvants in order to elicit a strong immune response. While mineral oil may suffice, the use of squalane emulsions stabilized by linear amphipathic polymers called pluronic polyols has been reported to be superior for eliciting an immune response. See Hunter, et al., Vaccine, 20 Suppl. 3, S7-12 (2002), which is incorporated herein in its entirety by reference. Furthermore, liposome formulations may be advantageously used to increase immunological response to proteins. See Lidgate, et al., Pharm. Research, 5, pg. 759-764 (1988); Hjorth, et al, Vaccine 15, 541-46 (1997), each of which is incorporated herein in its entirety by reference. General methods and protocols for administration of vaccines are also described in Plotkin and Orenstein, Vaccines, 4th ed.

The antigens provided by the invention are also useful for diagnostic purposes as well as for administration to induce immunity. A specific reaction to one or more, or two or more, or preferably three or more specific antigens identified by the above methods can be used to detect or quantify antibodies to the infectious agent, which allows rapid identification of the agent and the specific strain of the agent in an infected subject. An array of antigens can be used to very precisely distinguish a particular strain of an infectious agent. This permits detection of an infectious agent in an exposed subject even before symptoms have appeared. It permits determination of whether a subject has immunity to a specific infectious agent, so unnecessary immunization can be avoided. It also enables the identification of antibiotic-resistant bacterial infections or antiviral-resistant viral infections, for example, thus permitting a physician to avoid administering an ineffective drug and to quickly select an appropriate drug or therapy. Furthermore, it permits the user to identify specific disease states: the serum profile in a patient with chronic tuberculosis will be different from that in a patient with a new or active infection, and the disease state can thus be more precisely characterized using the antigens provided by the invention diagnostically.

The present invention also encompasses antibodies to proteins of the present invention and arrays of such antibodies. Antibodies may be made by any suitable means, for example, in laboratory animals such as rabbits, mice or domestic dogs. An antigen comprising a protein of the present invention may be mixed with incomplete Freund's adjuvant, alum adjuvant or with no adjuvant (PBS only) and injected into the laboratory animal, using one or more injections. Any form of the antigen can be used to generate the antibody that is sufficient to generate a specific antibody for a given antigen. The eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein).

As used herein, “antibodies” refers to both intact immunoglobulins and to immunologically reactive fragments of such antibodies, such as Fab, Fab′, F(ab′2), fragments, single-chain variable regions produced recombinantly—i.e., sFv forms, and any other fragments which are able specifically to recognize epitopes.

In some embodiments, a monoclonal antibody is preferred. Methods to generate monoclonal antibodies are well known in the art, and are generally described in Janeway, et al., Immunobiology, 5th ed., Garland Publishing, New York, N.Y. (2001), which is incorporated herein by reference. Methods to immobilize antibodies to produce arrays are also known in the art, such as application to a retentive surface such as nitrocellulose.

The antibodies can be screened for binding to normal or phenotypic variant forms of an antigenic protein. See e.g., ANTIBODY ENGINEERING: A PRACTICAL APPROACH (Oxford University Press, 1996), which is incorporated herein by reference. These monoclonal antibodies will usually bind with at least a Ka of about 1 μM, more usually at least about 300 nM, typically at least about 30 nM, often at least about 10 nM, frequently at least about 3 nM or better, usually determined by ELISA. Included in the definition of monoclonal antibodies are those that are chimeric forms (i.e., comprise portions of the heavy and light chains from different species) or are humanized or otherwise adapted to a particular subject by standard humanization or subject adaptation techniques.

The antibodies provided herein are useful in diagnostic applications, as well as in conferring passive immunity. They include isolated antibodies produced and at least partially purified using methods well known in the art. These antibodies can be used to detect or quantify the infectious agent from which the antigen was obtained; for example, they can be used to detect a bioweapon infectious agent in a subject or in a potentially contaminated material, because they can be very rapidly generated for a new strain. They may also be used to distinguish between strains of the infectious agent for therapeutic or epidemiology purposes, or to identify specific strains such as those that are sensitive to or insensitive to specific drugs. Arrays of the antibodies are useful for identifying a specific strain of an infectious agent. The antibodies are also useful reagents for antigen purification.

The following examples are offered to illustrate but not to limit the invention. In these examples, the vaccinia strain used was the WR strain. Sequences of the open reading frames of the genome of this strain are deposited at GenBank with the designations VACWR followed by a number. A list of the loci of the open reading frames is found in Table 8, which follows these examples. The orthologs of the open reading frames listed in Table 8 for the WR strain that are present in the Copenhagen strain are also characterized by their sequences in GenBank where they have the designations shown in the second column of Table 8.

It will be seen that one of the loci in the WR strain, VACWR148, does not have a corresponding ortholog in the Copenhagen strain; it corresponds in part to the antigen having the designation A29L in Variola major and was initially identified as such. On closer scrutiny, WR148 shows a strong immuno-dominant antigenic response but does not map to a single gene in related species. Rather, the WR146, WR147, WR148, and WR149 genes correspond to an A-type inclusion protein group or ATI locus proteins. The ATI locus proteins correspond to A26L and A27L in cowpox, and to A26L, A27L, A28L, A29L and A30L in variola.

In the examples and in the claims, the nomenclature corresponding to the Copenhagen ortholog is used for the other genes and gene products, and ATI locus genes or ATI locus proteins for the VACWR148 antigens. The correspondence to the WR strain used in the example can be found in Table 8.

### EXAMPLE 1

### Preparation of Vector and Inserts

A linear T7 vector encoding an N-terminal histidine tag and a C-terminal HA tag was generated by extensive restriction digestion followed by PCR; this procedure reduced the amount of residual circular vector and background colonies to nearly zero when it is transformed without complementary insert into chemically competent E. coli.

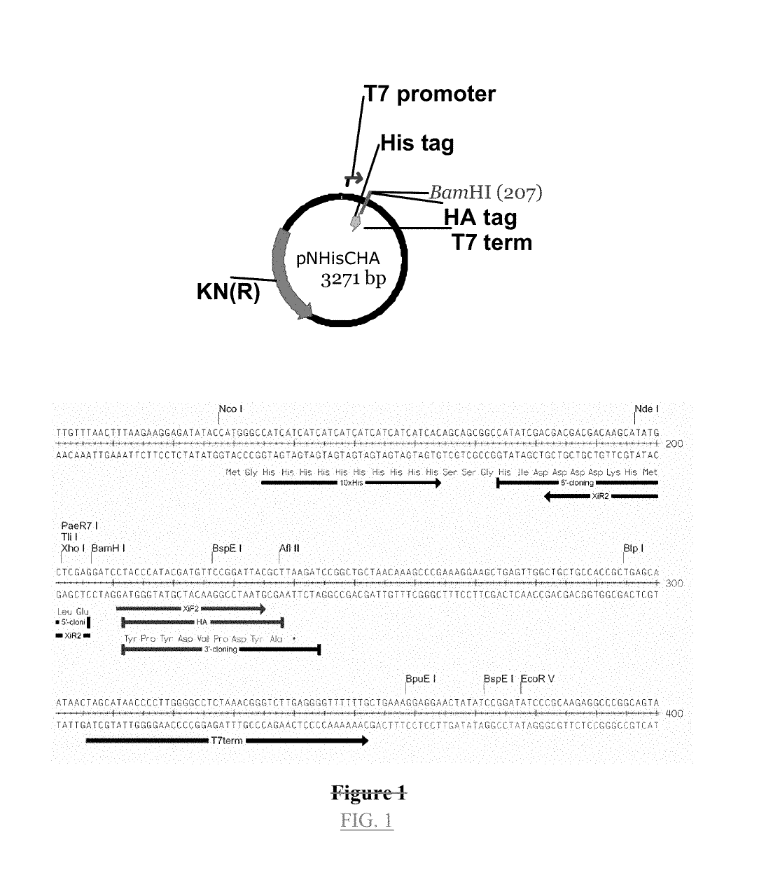

The plasmid used to generate the linear recombination vector pXT7, is shown in FIG. 1. This vector contains a T7 promoter, followed by ATG start codon, a 10× histidine sequence, a spacer sequence in front of the first codon of the open reading frame to be cloned, a BamH1 site, and a T7 terminator. The vector was double digested at the BamH1 site to eliminate residual circular vector, since incompletely digested vector creates background colonies that lack insert. This linearized vector was amplified by PCR to generate inventory of the linear recombination vector. Each batch of linear vector was transformed into competent E. coli to verify that it was not producing background colonies.

In more detail, plasmid pXT7 (10 μg; 3.2 kb, KanR) was linearized with BamH1 (0.1 μg/μl DNA, 0.1 mg/ml BSA, 0.2 U/μl BamH1, 37° C., 4 h; additional BamH1 was added to 0.4 U/μl, 37° C., overnight). The digest was purified (Qiagen PCR purification kit), quantified by fluorometry and verified by agarose gel electrophoresis (1 μg). One nanogram of this material was used to generate the linear acceptor vector in a 50 μl-PCR (Primers, 0.5 μM each: 5′CTACCCATACGATGTTCCGGATTAC, 5′CTCGAGCATATGCTTGTCGTCGTCG; 0.02 U/μl Taq DNA polymerase [Fisher Scientific, buffer A]; 0.1 mg/ml gelatin [Porcine, Bloom 300; Sigma, G-1890]; 0.2 mM each dNTP; initial denaturation: 95° C., 5 min; 30 cycles: 95° C., 0.5 min/50° C., 0.5 min/72° C., 3.5 min; final extension: 72° C., 10 min). The PCR product was visualized by agarose gel electrophoresis (3 μl), purified (Qiagen PCR purification kit), and quantified by fluorometry using picogreen (Molecular Probes) according to the manufacturer's instructions. Each batch of linear acceptor vector was checked for background KanR transformants (no KanR transformant per 40 ng).

ORF's from vaccinia virus and F. tularensis were amplified using gene specific primers containing 33 nucleotide extensions complementary to the ends of the linear T7 vector.

One to ten nanograms genomic DNA were used as template in a 50 μl-PCR: Primers, 0.5 μM each (5′CATATCGACGACGACGACAAGCATATGCTCGAG [20-mer ORF specific at 5′-end]; 5′ATCTTAAGCGTAATCCGGAACATCGTATGGGTA [20-mer ORF specific at 3′-end]); 0.02 U/μl Taq DNA polymerase [Fisher Scientific, buffer A]; 0.1 mg/ml gelatin [Porcine, Bloom 300; Sigma, G-1890]; 0.2 mM each dNTP; initial denaturation: 95° C., 5 min; 30 cycles: 20 sec at 95° C., 0.5 min at 50° C., 1 min per 1 kb at 72° C., 1 to 3 min on average based on ORF size; final extension: 72° C. for 10 min). Those PCR products more difficult to produce were re-amplified using 0.5 min annealing at 45 and 40° C. instead of 50° C. The PCR products were purified (Qiagen PCR purification kit), quantified by fluorometry using picogreen (Molecular Probes, Eugene Oreg.) and visualized to validate size and purity by agarose gel electrophoresis.

Each open reading frame was amplified from genomic template using gene specific primers. The 5′ oligonucleotide contained 53 nucleotides; of these 33 nucleotides comprise the 5′ universal end sequence and the other 20 nucleotides make up the gene-specific sequence. The first start codon, ATG, is upstream of the polyhistidine tag on the linear vector, and each open reading frame also begins with ATG. The 3′-custom oligonucleotide also contains 53 nucleotides; of these, 33 comprise the 3′ universal end sequence and the other 20 nucleotides are specific to the gene-of-interest. A stop codon sequence, TTA, was added to the end of the gene sequence to achieve translational termination of the expressed gene.

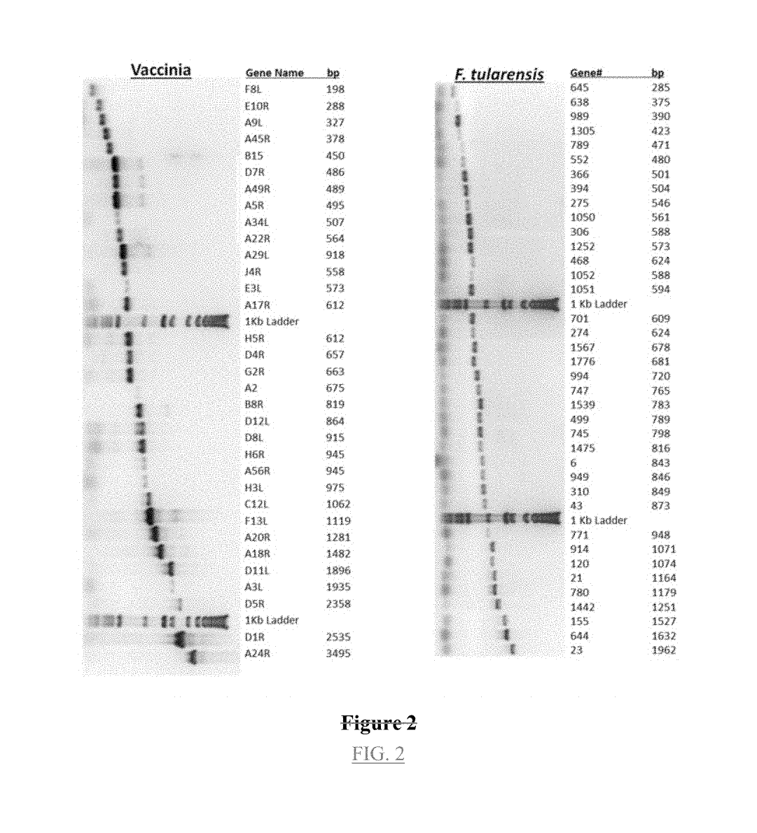

The primers are shown in FIG. 1, and a gel showing a set of cleaned PCR products amplified from vaccinia and F. tularensis is shown in FIG. 2. For genes shorter than 1,000 bp the success rate for getting the predicted PCR product was greater than 99%. For these short genes, failures could be recovered by ordering new primers. Twenty-eight (28) out of32 genes between 1,000 and 2,000 bp (81%) could be amplified using the procedures detailed in the methods section. Only 3 out of 8 genes greater than 2,000 bp could be amplified by these methods. These longer genes can be amplified as overlapping fragments, or different PCR conditions can be applied that favor amplification of longer products.

### EXAMPLE 1A

Applying these methods to the vaccinia virus required preparation of primers for 213 genes, from which 211 PCR products were isolated (>99%). All 211 of these were cloned, and 181 of the products were submitted for sequencing; 93% (169 out of 181) provided the predicted sequence.

EXAMPLE 1B

Similarly, applying the methods to P. falciparum required preparation of primers for 720 genes. From these, 462 PCR products were obtained (64%), and 266 clones were produced (58%). A set of these (63) were submitted for sequencing, with 97% giving the expected sequence.

EXAMPLE 1C

The above methods were applied to Mycobacterium tuberculosis for which primers for 108 genes were prepared. From these, 87 PCR products were obtained (80%) and 80 clones were produced (92%), each of which had an anti-His tag on one end and an anti-HA tag on the other. Sequencing confirmed that 70 out of 79 tested (88%) contained the expected sequence. In most of the proteins produced, both the His and HA tags were accessible for binding, but in a number of cases, only one tag was bound; generally, where only one was accessible, it was the His tag that remained accessible for binding, and the HA epitope tag that was inaccessible.

This method was expanded to express 312 genes from Mycobacterium tuberculosis H37Rv, out of a genome of about 4,000 genes.

### EXAMPLE 1D

The above methods were applied to F. tularensis for which primers for 1933 genes were prepared. From these, 1842 PCR products were obtained (95%) and 1720 clones were produced (93%). Sequencing of 684 of these showed that 643 (94%) contained the expected sequence.

### EXAMPLE 2

### In Vivo Recombination and Colony Selection

Mixtures of PCR amplified ORF's and linear T7 vector of Example 1 were mixed and introduced into chemically competent E. coli, resulting in transformed colonies containing plasmid with insert. This high efficiency recombination cloning method resulted in in-frame directional insertion of ORF.

The competent cells were prepared in our laboratory by growing DH5a cells at 18° C. in 500 ml SOB medium (2% tryptone, 0.5% yeast extract, 10 mM NaCl, 2.5 mM KCl, and 20 mM MgSO4) to an optical density of 0.5-0.7 O.D. The cells were washed and suspended in 10 ml pre-chilled PCKMS buffer (10 mM PIPES, 15 mM CaCl₂, 250 mM KCl, 55 mM MnCl₂, and 5% sucrose, pH 6.7) on ice and 735 μl DMSO was added dropwise with constant swirling. The competent cells were frozen on dry ice ethanol in 100 μl aliquots and stored at −80° C.

Each transformation consisted of: 10 μl competent DH5α (prepared as above in our laboratory with efficiency of 10⁹ cfu/mg of supercoiled plasmid DNA) and 10 μl DNA mixture (40 ng PCR-generated linear vector, 10 ng PCR-generated ORF fragment; molar ratio 1:1, vector: 1 kb ORF fragment). The mixture was incubated on ice, 45 min; heat shocked (42° C., 1 min); chilled on ice, 1 min; mixed with 250 μl SOC medium (2% tryptone, 0.55% yeast extract, 10 mM NaCl, 10 mM KCl, 10 mM MgCl₂, 10 mM MgSO₄, 20 mM glucose); incubated 37° C., 1 h; diluted into 3 ml LB (Luria Bertani Medium) supplemented with 50 μg kanamycin/ml (LB Kan 50), and incubated with shaking overnight. Single colonies were obtained from the overnight culture by streaking on LB Kan 50 agar. From each transformation, 2-3 colonies were selected for further analysis. Plasmid DNA obtained from Qiagen miniprep was visualized by gel electrophoresis for selection of clones with insert.

Transformation of the DH5a competent cells was accomplished with a mixture of PCR fragments and linear vector in a molar ratio of 1:1 and with 50 ng of total DNA used in the transformation. The competent cells were transformed, grown overnight and observed for turbidity due to bacterial growth before plating and colony selection. Under these conditions cloning efficiency was >90%, but if the cells were plated on the day of transformation the observed success rate was lower. The rate of successful transformation progressively declined as the total DNA used for transformation was reduced to 25 and 10 ng (not shown).

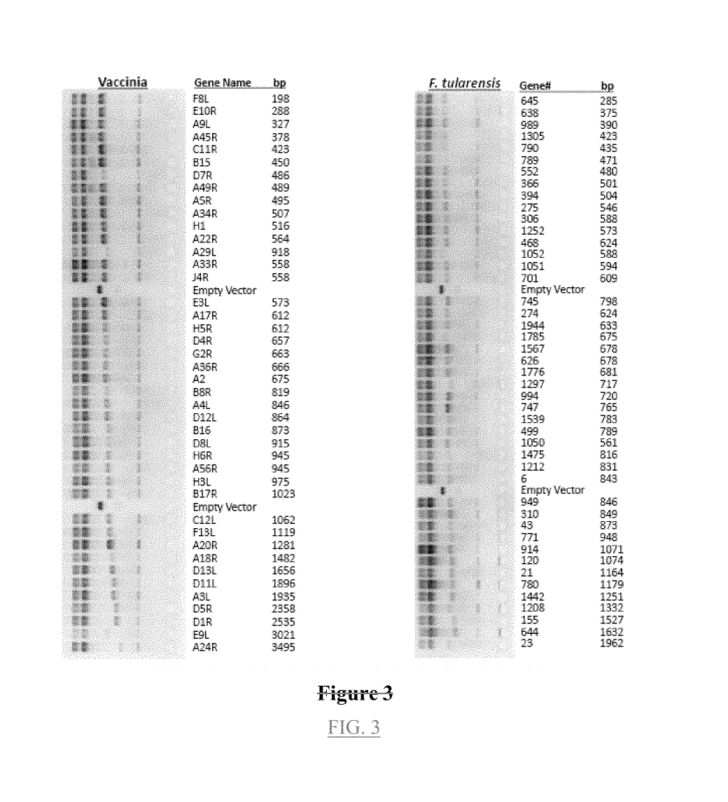

FIG. 3 shows a “cracking gel” (phenol-chloroform lysed bacteria showing total nucleic acid) from overnight cultures using the PCR fragment shown in FIG. 2. The top band on these gels is genomic DNA, and the bottom two bands are heavy and light ribosomal RNA and the central band is the plasmid formed by recombination with linear vector and PCR fragment. Empty vector is included on this gel for reference. Out of the 87 plasmids shown in this figure, only 3 lack insert of the appropriate size.

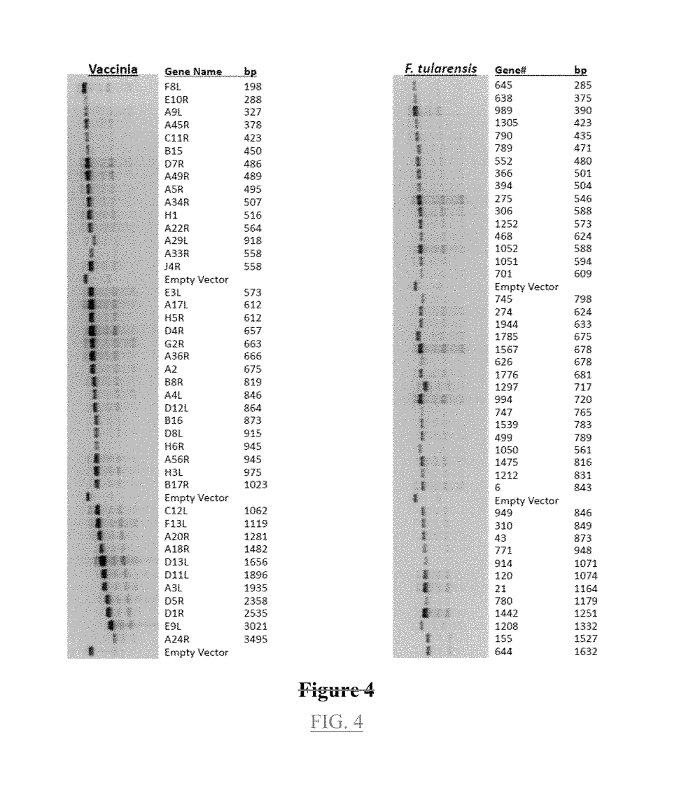

The overnight cultures shown in FIG. 3 were streaked on agar plates, 2 colonies selected, grown and miniprepped. Minipreps of single colonies derived from the overnight cultures are shown in FIG. 4. The purified plasmids were sequenced to verify the fidelity of the recombination product. The majority of inserts sequenced accurately according to the genome sequence databases. 74% had no mutations, 20% had single point mutations and 6% had more than one point mutation. 41% of the point mutation were A to G; the remaining mutations were randomly distributing among the other 11 types of possible point mutations.

### EXAMPLE 3

### In Vitro Transcription and Translation Detection of Protein

The proteins encoded on the plasmids shown in FIG. 4 were expressed in an E. coli based cell-free in vitro transcription/translation system that was supplemented with T7 RNA polymerase. Plasmid templates 0.5 μg of each miniprep were prepared using the Qiagen miniprep kits, and including the “optional” step which contains protein denaturants to deplete RNAse activity. If this step is not included, the level of expression in the in vitro transcription/translation reaction will be low and inconsistent. In vitro transcription/translation reactions (RTS 100 E. coli HY kits from Roche) with 25 μl reaction volumes were set up in 0.2 ml PCR 12-well strip tubes and incubated for 5 h at 30° C. according to the manufacturer's instructions. Western blots were performed using mouse anti-histidine antibody and goat anti-mouse antibody conjugated to alkaline phosphatase.

For the results shown in FIG. 5, 50 different F. tularensis and vaccinia plasmids were incubated in the in vitro transcription/translation reaction for 4 hours, the product was run on SDS polyacrylamide gels, and the gels were blotted and probed with anti-polyhistidine antibody. The Western blots in FIG. 5 show expression of the histidine tagged products of the predicted molecular weights and only 3 out of 50 plasmids were negative.

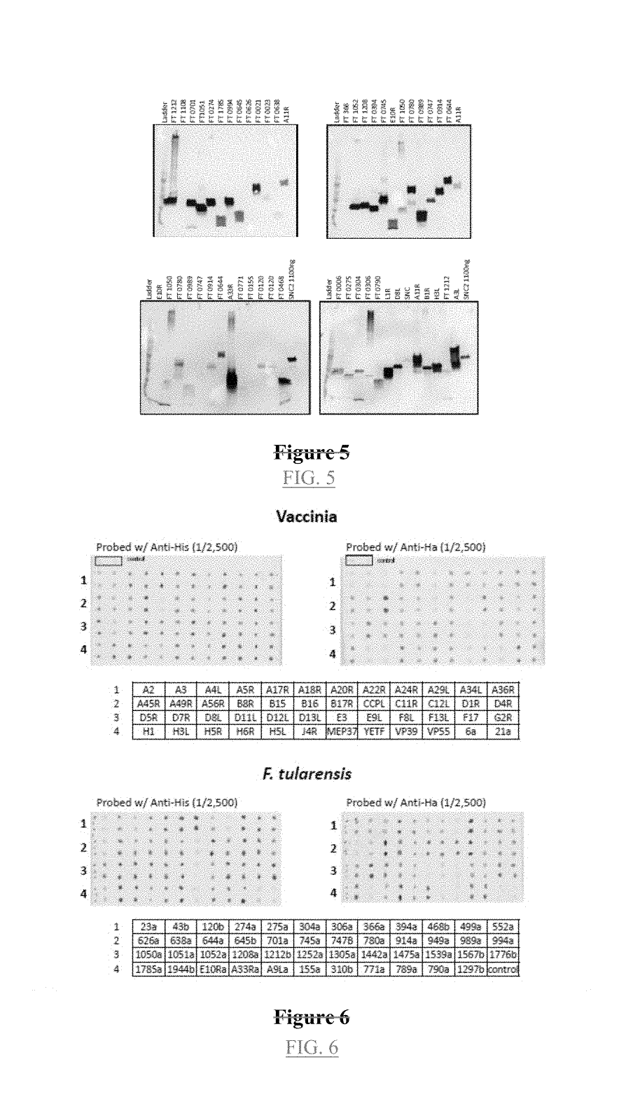

Non-denatured proteins from the cell-free reactions could also be detected on dot-blots. (FIG. 6) One microliter of each in vitro transcription/translation reaction was spotted directly onto nitrocellulose, without SDS denaturation, and the dot-blots were probed with either anti-histidine or anti HA antibodies. The reaction products from 50 vaccinia virus clones and 45 F. tularensis clones are shown (FIG. 6). When the dot-blots were probed with anti-histidine antibody, one of the vaccinia reactions and 3 of the F. tularensis reactions were not above background. There were a larger number of negative reactions when the dot-blots were probed with anti-HA antibody, presumably indicating that this epitope is more frequently concealed within the 3-dimensional structure of the non-denatured protein, since electrophoresis and Western blot analysis did not reveal abundant premature protein product due to early stop during translation. (Further details of preparing dot-blots are presented in Example 4.)

### EXAMPLE 4

### Microarrays and Serological Screening

Commercially available Vaccinia Immune Globulin (VIG) from Cangene Corp (Winnipeg, Canada) was used. VIG is the immunoglobulin fraction of hyperimmune sera pooled from multiple donors. It is used as an emergency therapy for people undergoing systemic viraemia and other adverse reactions to vaccinia vaccination.

For immuno-dot-blots, 0.3 Ill volumes of whole RTS reactions were spotted manually onto nitrocellulose membranes and allowed to air dry prior to blocking in 5% non-fat milk powder in TBS-Tween. Blots were probed with VIG, diluted to 1/1,000 in blocking buffer with or without 10% E. coli lysate. Three different batches of VIG were used: lot #1730204 (56 mg/ml), lot #1730208 (53 mg/ml) and lot #1730302 (56 mg/ml). Bound human antibodies were detected by incubation in alkaline phosphatase-conjugated goat anti-human IgA+IgG+IgM (H+L) secondary antibody (Jackson ImmunoResearch) and visualized with nitro-BT developer. Routinely, dot-blots were also stained with both monoclonal anti-polyhistidine (clone His-1; Sigma H-1029) and with monoclonal rat anti-hemagglutinin (clone 3F10; Roche 1 867 423), followed by AP-conjugated goat anti-mouse IgG (H+L) (BioRad) or goat anti-rat IgG (H+L) secondary antibodies (Jackson ImmunoResearch), respectively, to confirm the presence of recombinant protein.

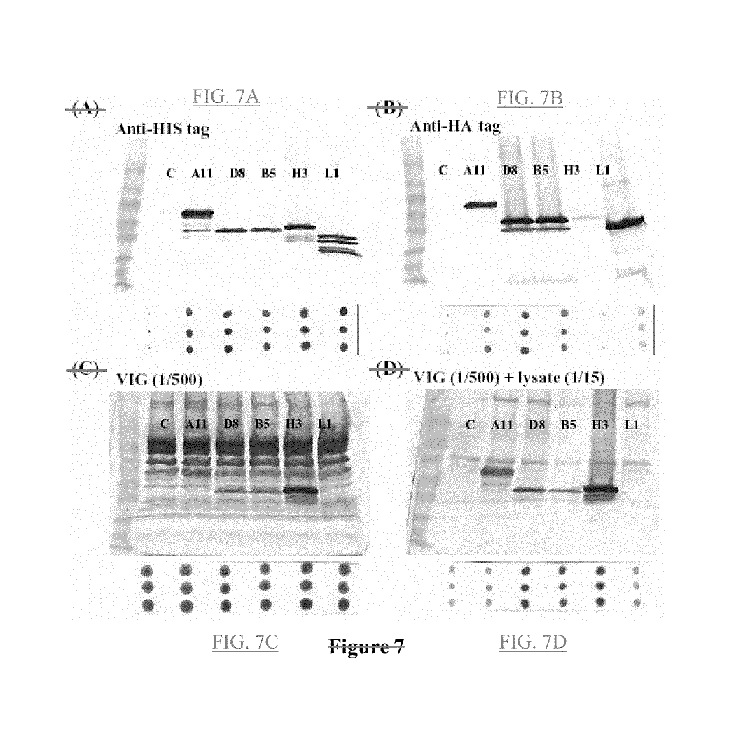

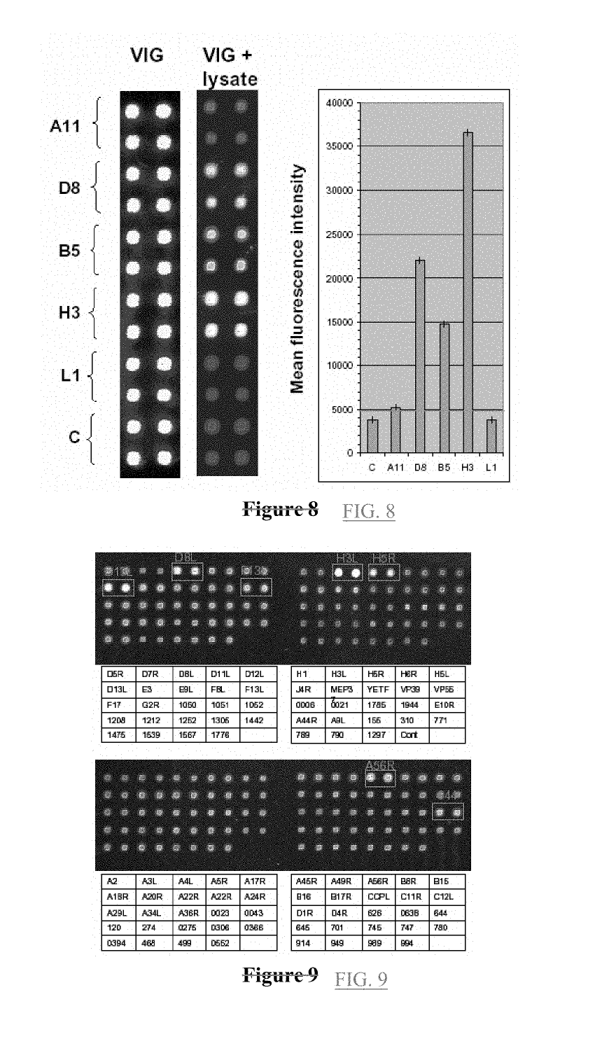

In vitro transcription/translation reactions set up in a 25 μl scale, and control reactions using non-recombinant expression plasmid as the template are also set up to control for the presence of E. coli antigens are used. Immediately after the end of the 5 h synthesis reaction, the proteins were either spotted or arrayed onto nitrocellulose substrates without further purification, or held at 4° C. for no more than 12 h prior to printing. Spotting of RTS reactions was under non-denaturing conditions, and without further purification (FIGS. 7A-7D). Antibodies to E. coli are found in high titer in human sera and VIG and unless blocked cause high background staining that masks any antigen-specific responses. This is overcome either by removal of the anti-E. coli reactivity using E. coli proteins immobilized on nitrocellulose membranes, or by blocking the antibodies by the inclusion of 10% E. coli lysate in the serum or VIG. In practice, we observed no difference in the effect of adsorption against immunoblots compared to blocking by the addition of lysate (data not shown). The latter technique was therefore adopted as the routine method of blocking the E. coli background staining because of its compatibility with high throughput screening and the economic use of human serum it allows (typically 2-3 μl per microarray). When lysate is included the intensity of the spot in the control reaction is dramatically reduced resulting in a stronger signal to noise ratio against antigenic vaccinia proteins. Notice also that the reactivity of VIG to A11L is conformation dependent. This particular antigen is readily recognized in the Western blot but not in the non-denaturing format of the dot-blot.

### EXAMPLE 5

### Microarrays

FIG. 8 shows a pilot microarray using the same RTS reactions used for the immuno-dot-blot depicted in FIG. 7. For microarrays, 15 μl volumes were first transferred to 384 well plates, centrifuged 1,600×g to pellet any precipitate, and supernatant printed without further purification onto nitrocellulose-coated FAST™ glass slides (Schleicher & Schuell Bioscience) using an Omni Grid 100 microarray printer (Gene Machines). For all staining, slides were first blocked for 1 h in protein array blocking buffer (Schleicher & Schuell) and stained with the same primary and secondary antibodies as for the dot-blots (with Cy3 conjugated secondary antibodies from Jackson) and scanned in a laser confocal scanner. Fluorescence intensities were quantified using QuantArray software (GSI Lumonics, Inc). VIG has high titers of anti-E. coli antibodies that mask any antigen-specific responses when using whole RTS reactions on dot-blots and arrays. This was overcome by the adsorption of VIG against immunoblots of E. coli lysates, or by the addition of E. coli lysate to the VIG. In the former method, E. coli was solubilized in SDS PAGE sample buffer and the lysate resolved on preparative gels prior to transfer to Optitran nitrocellulose membranes (Schleicher & Schuell). The blots were then cut into small (5×5 mm) pieces and blocked in 5% non-fat milk powder for 1 h. The pieces were then rinsed and placed into VIG previously diluted to 1/1000 in blocking buffer, and incubated for 1 h with constant agitation. E. coli lysate was produced from a 1 liter stationary phase culture of E. coli (DH5) resuspended in 25 ml TBS-Tween and sonicated with a 2 cm diameter probe. One ml aliquots were stored at −80° C.

In vitro transcription/translation reactions were printed, without purification, onto nitrocellulose-coated glass slides and probed with VIG with and without 10% E. coli lysate. The control spots consist of RTS reactions with non-recombinant expression plasmid as the vector. An arbitrary “cut-off”, over which staining can be considered positive, was established by calculating the mean and standard deviation of the fluorescence intensity of the control spots. As can be seen when lysate is present in the VIG, the same proteins that were detected in the immuno-dot-blot are also detected by microarray. The fluorescently conjugated secondary antibodies provide a wider range of signal intensities than seen with the immuno-dot-blots. Moreover the microarrays also appear to give greater sensitivity than the immuno-dot-blots, since we have observed several cases where proteins that were detected in arrays were below the threshold of detection in the dot-blots (not shown).

FIG. 9 shows a larger microarray of 96 vaccinia and F. tularensis proteins, plus one control reaction, expressed in the PCR Express™ platform. The array shows seven proteins strongly recognized by VIG, of which six are vaccinia proteins. Of these, four (H3L, DBL, A56R and FI3L) are viral envelope antigens that are accessible to antibodies on the surface of the intact virus particle. Thus the detection of proteins in this system shows a high degree of antigen specificity and biological relevance. The non-denatured format has the added advantage that the proteins are likely to preserve their conformation-dependant epitopes.

### EXAMPLE 6

### Preparation of Plasmids from Transformation Mixtures

Rather than selecting individual colonies for further assessment as in Examples 2-5, the transformation mixture, obtained as described in Example 2 was used as the source of plasmids containing the desired inserts. As above, each transformation consisted of: 10 μl competent DH5α and 10 μl DNA mixture (40 ng PCR-generated linear vector, 10 ng PCR-generated ORF fragment from vaccinia; molar ratio 1:1, vector: 1 kb ORF fragment). The mixture was incubated on ice, 45 min; heat shocked (42° C., 1 min); chilled on ice, 1 min; mixed with 250 μl SOC medium (2% tryptone, 0.55% yeast extract, 10 mM NaCl, 10 mM KCl, 10 mM MgCl₂, 10 mM MgSO₄, 20 mM glucose); incubated 37° C., 1 h; diluted into 3 ml LB (Luria Bertani Medium) supplemented with 50 μg kanamycin/ml (LB Kan 50), and incubated with shaking overnight. The plasmid was isolated and purified from this culture, without colony selection. The resulting plasmid templates were translated substantially as described in the foregoing examples and transferred to immuno-dot-blots as follows:

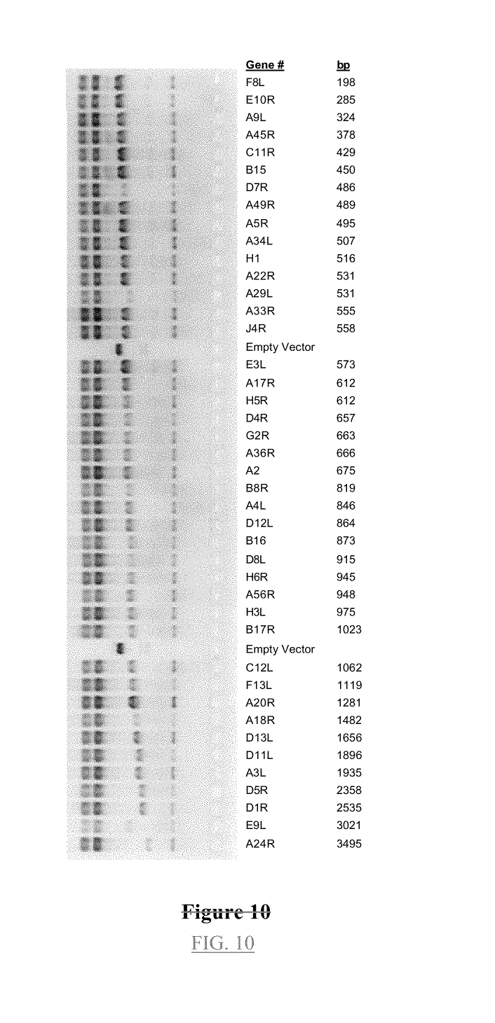

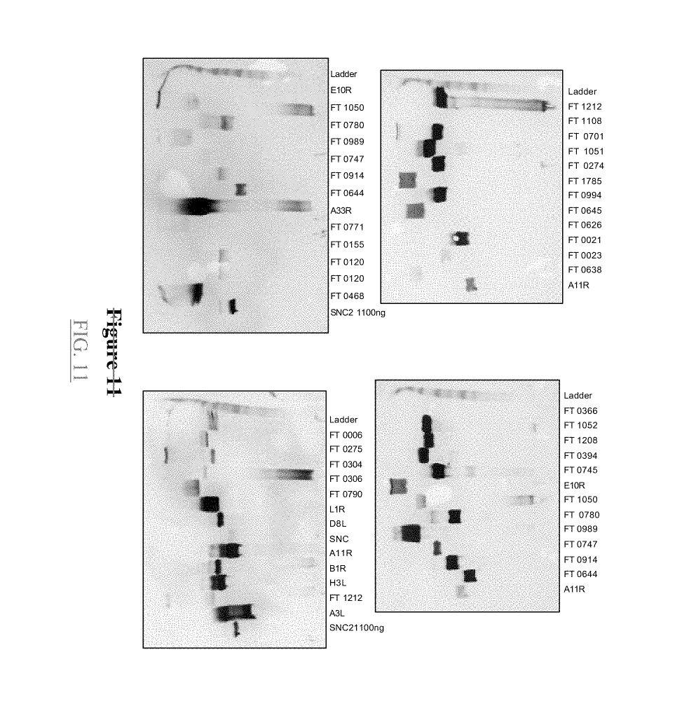

Plasmid templates used for in vitro transcription/translation were prepared using the Qiagen miniprep kits, including the “optional” step which contains protein denaturants to deplete RNase activity. If this step is not included, the level of expression in the in vitro transcription/translation reaction was low and inconsistent. FIG. 10 shows a “cracking gel” (phenol-chloroform lysed bacteria showing total nucleic acid) from overnight cultures using the PCR fragments from vaccinia. The top band on these gels (oriented to the right) is genomic DNA, the bottom two bands are 23S and 16S ribosomal RNA, and the central band is the plasmid formed by recombination with linear vector and PCR fragment. Empty vector is included on this gel for reference. Out of the 42 plasmids shown in this figure, only 1 (E9L) lacks insert of the appropriate size. To calibrate the efficiency of the overall system a test set of genes from Francisella tularensis were amplified cloned and expressed. Out of 1,933 genes attempted, 96% were successfully amplified and 93% of those were successfully cloned.

In vitro transcription/translation reactions (RTS 100 E. coli HY kits from Roche) with 25 μl reaction volumes were set up in 0.2 ml PCR 12-well strip tubes and incubated for 5 h at 30° C. according to the manufacturer's instructions. The proteins encoded on the T7 plasmids representing a set of 8 vaccinia and 40 F. tularensis proteins were expressed in an E. coli based cell-free in vitro transcription/translation system that was supplemented with T7 RNA polymerase. The 25 μl in vitro transcription/translation reactions were incubated for 4 hours at 37° C., the crude unpurified reactions were resolved on SDS polyacrylamide gels, and the gels were blotted and probed with anti-polyhistidine antibody (FIG. 11). The Western blots show expression of the histidine tagged products of the predicted molecular weights. Three out of the 48 reactions were too weak to score as positive.

For immuno-dot-blots, 0.3 μl volumes of whole RTS reactions were spotted manually onto nitrocellulose membranes and allowed to air dry prior to blocking in 5% non-fat milk powder in TBS containing 0.05% Tween 20. Blots were probed with vaccinia immune globulin (VIG) from Cangene Corporation (Winnipeg, Manitoba, Canada) diluted to 1/1000 in blocking buffer with or without 10% E. coli lysate. Three different batches of VIG were used: lot #1730204 (56 mg/ml), lot #1730208 (53 mg/ml) and lot #1730302 (56 mg/ml). Bound human antibodies were detected by incubation in alkaline phosphatase-conjugated goat anti-human IgA+IgG+IgM (H+L) secondary antibody (Jackson ImmunoResearch) and visualized with nitro-BT developer. Routinely, dot-blots were also stained with both monoclonal anti-polyhistidine (clone His-1; Sigma H-1029) and with monoclonal rat anti-hemagglutinin (clone 3F10; Roche 1 867 423), followed by AP-conjugated goat anti-mouse IgG (H+L) (BioRad) or goat anti-rat IgG (H+L) secondary antibodies (Jackson ImmunoResearch), respectively, to confirm the presence of recombinant protein. For microarrays 10 μl of 0.125% Tween 20 was mixed with 15 μl RTS reaction (to give a final concentration of 0.05% Tween), and 15 μl volumes were transferred to 384-well plates. The plates were centrifuged 1600×g to pellet any precipitate, and supernatant printed without further purification onto nitrocellulose-coated FAST™ glass slides (Schleicher & Schuell Bioscience) using an Omni Grid 100 microarray printer (Gene Machines). For all staining, slides were first blocked for 30 mins in protein array blocking buffer (Schleicher & Schuell) and stained with the same primary and secondary antibodies as for the dot-blots (with Cy3 conjugated secondary antibodies from Jackson) and scanned in a laser confocal scanner. Fluorescence intensities were quantified using QuantArray software (GSI Lumonics, Inc). VIG has high titers of anti-E. coli antibodies that mask any antigen-specific responses when using whole RTS reactions on dot-blots and arrays. This was overcome by the adsorption of VIG against immunoblots of E. coli lysates, or by the addition of E. coli lysate to the VIG. In the former method, E. coli was solubilized in SDS PAGE sample buffer and the lysate resolved on preparative gels prior to transfer to Optitran nitrocellulose membranes (Schleicher & Schuell). The blots were then cut into small (5×5 mm) pieces and blocked in 5% non-fat milk powder for 1 h. The pieces were then rinsed and placed into VIG previously diluted to 1/1000 in blocking buffer, and incubated for 1 h with constant agitation. E. coli lysate was produced from a lliter stationary phase culture of E. coli (DH5α) resuspended in 25 ml TBS-Tween and sonicated with a 2 cm diameter probe. One ml aliquots were stored at −80° C. Mouse sera, which lack endogenous anti-E. coli reactivity, do not require pre-treatment with E. coli lysate to reduce background.

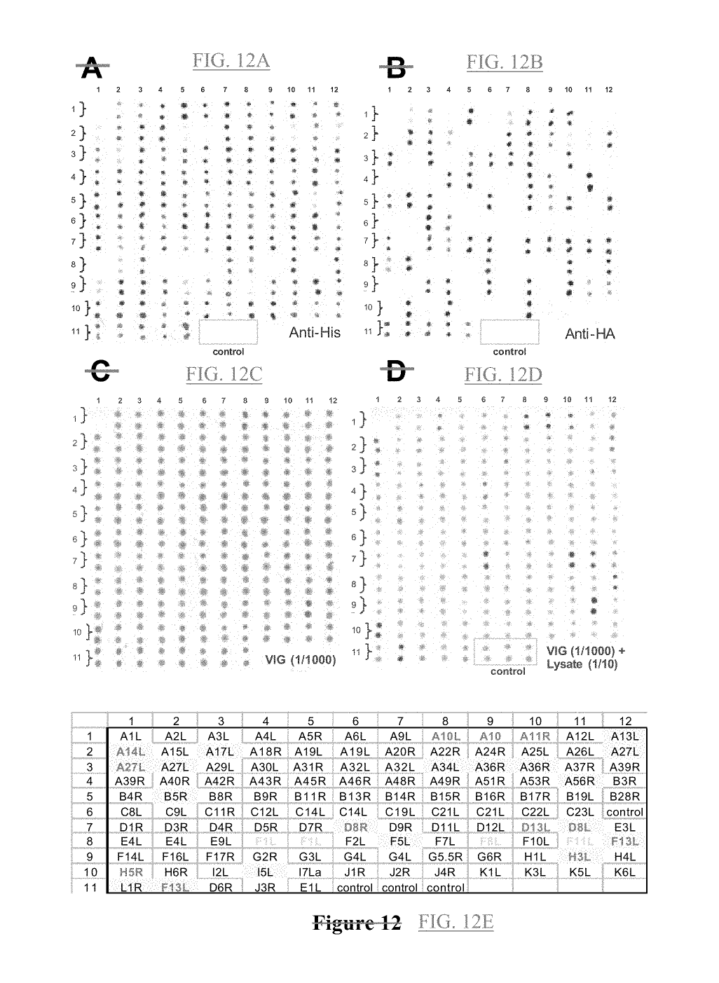

Non-denatured proteins from the cell-free reactions could also be detected on immuno-dot-blots (FIGS. 12A-12D). 128 plasmids encoding 112 different vaccinia proteins were expressed in vitro and one microliter of each of the unpurified reactions was spotted in duplicate onto nitrocellulose. The open reading frame of each gene is designed to include an N-terminal 1O× histidine (HIS) tag and a C-terminal hemagglutinin tag (sequence YPYDVPDYA). A control reaction (‘c’) lacking plasmid template was also set up; if empty vector is used a positive signal was observed due to a small 1O× histidine positive fragment produced (data not shown). Membranes were probed with either anti-HIS tag antibody (FIG. 12A), anti-HA tag antibody (FIG. 12B), vaccinia immune globulin (VIG) (FIG. 12C), or VIG+10% E. coli lysate (FIG. 12C). The anti-HIS and HA tag antibodies show no cross-reactivity with other proteins in the in vitro reactions, and are therefore used routinely for monitoring the expression of large numbers of reactions. Out of 112 different proteins expressed, only 3 were negative for both the HIS (Panel 12A) and HA (Panel 12B) tags. To evaluate the overall efficiency of expression, 390 cloned F. tularensis genes were expressed, the reactions were spotted onto nitrocellulose and probed with either anti-Histidine or anti-HA antibody. 82% of the reactions were HA positive, 84% were 10x histidine positive, 73% were both histidine and HA positive, and 7% were HA and histidine negative.

It is apparent from the blot in panel12C that VIG has high titers of anti-E. coli antibody, masking any reactivity to vaccinia proteins. However, the addition of E. coli lysate to VIG (panel 12D) reduces this background to a level such that the detection of the vaccinia protein is possible. Positive proteins on this blot were, A10L, A27L, D8L, D13L, F13L, H3L & HSR, highlighted in red in the caption.

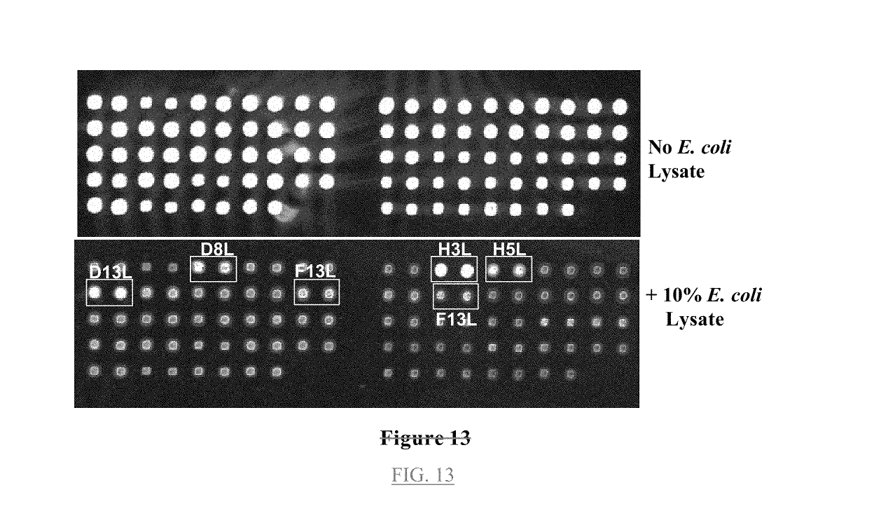

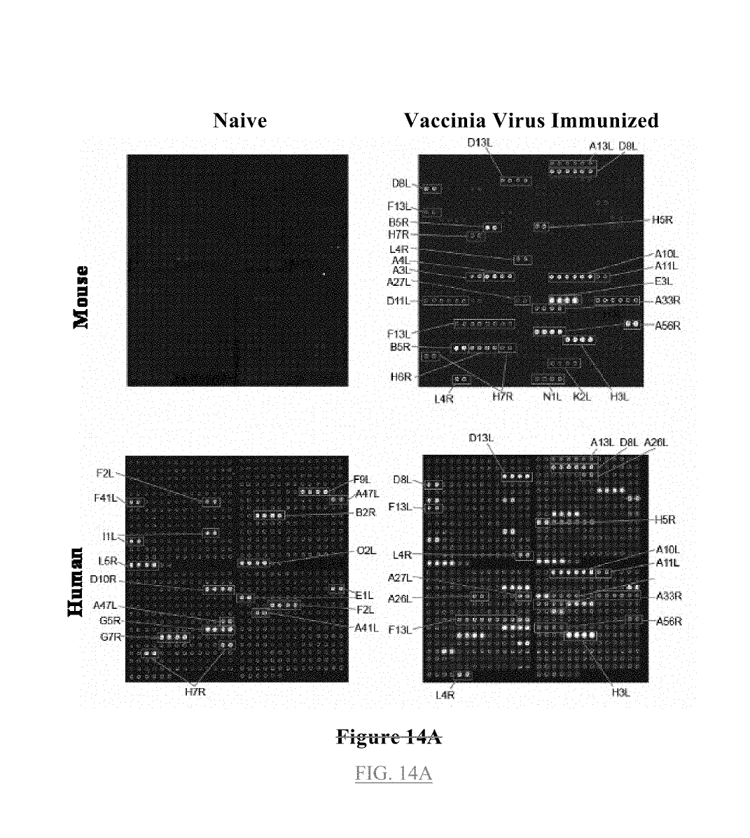

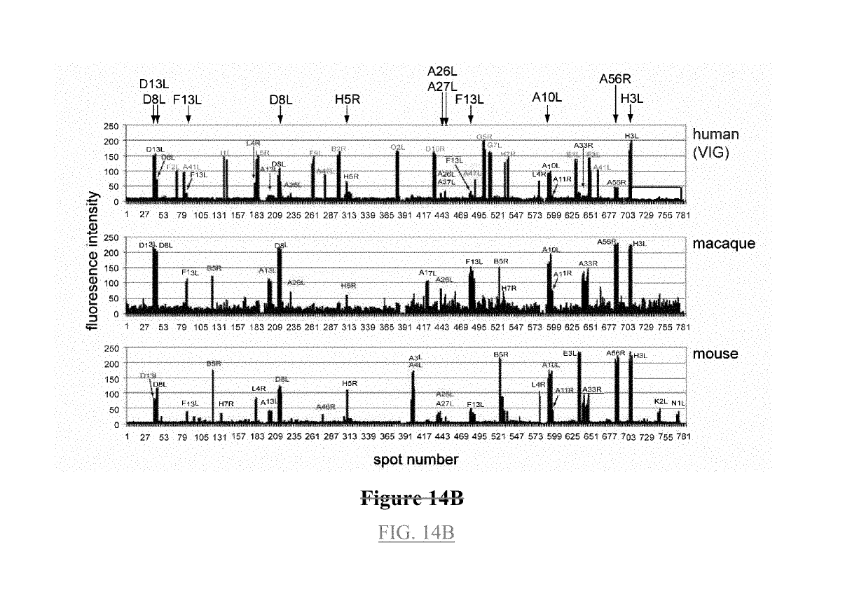

E. coli lysate treatment of serum was also effective to reduce E. coli background reactivity on microarrays. A pilot microarray consisting of 23 vaccinia and 22 F. tularensis proteins probed with VIG, with and without E. coli lysate is shown in FIG. 13. The effect of high titers of anti-E. coli antibody, as seen in the dot-blot in FIG. 12C, is also obvious on microarrays (FIG. 13, top array). This high background that is also present in the control preparations masks specific reactivity to vaccinia proteins. Addition of 10% E. coli lysate to VIG before probing the microarray reduced the E. coli background revealing the specific reactivity (FIG. 13, lower panel). The array shows 5 vaccinia proteins strongly recognized by VIG (boxed), D13L, D8L, F13L, H3L & H5L.

FIGS. 14A-14B show results from an array consisting of 194 proteins estimated to represent >95% of the complete vaccinia virus proteome. This array was screened with human vaccinia immune globulin (VIG), and sera from mice and macaques before and after vaccination with vaccinia virus. FIG. 14A shows that naive non-immunized mice completely lack reactivity against all of the proteins on the array, but sera from vaccinia virus immunized mice react with a subset of the antigens on the array. Unlike naive mice, non-immunized humans react with a subset of antigens on the array, but following immunization with vaccinia virus another subset of reactive antigen develop. Quantification of the data is represented graphically in the upper panel of FIG. 14B. VIG recognizes 26 different proteins, of which 13 are also seen by sera from vaccinia-naive individuals and are therefore thought to represent non-specific cross-reactions by antibodies to other environmental antigens. The remaining 13 are antigens specifically recognized by antibodies raised during vaccinia immunization. Similar profiles are also seen in sera from macaque and mouse (FIG. 14B). While there are species-specific responses (for example, A3L or A4L in mice only) there are many recognized in common by humans and either animal model, and ten proteins recognized by all three species (Table 1). These particular antigens would be priority candidates for the preclinical testing of a vaccine for use in humans. Overall, responses to viral structural proteins dominate the response, with more than half of these being envelope proteins (Table 1). The proteins that were seropositive included those with and without transmembrane domains, with and without signal peptides and PI ranges from 4-10. Moreover, several of these proteins have been previously reported to produce humoral responses in animals and humans, whereas others have not.

The antigens in Table 1 are all proteins from the Western Reserve (WR) strain, but are identified herein by the name of their nearest ortholog in the Copenhagen strain of vaccinia virus, since the protein functions are better characterized in that strain. Nevertheless, sequences for each of the ORFs and for the encoded proteins from the WR strain are available in the GenBank database, which is available online at the web address www.ncbi.nlm.nih.gov/gquery/gquery.fcgi. The descriptions set forth in Table 1 match those in the database. The protein and gene sequences for the WR strain are in the Vaccinia WR genome, and can be located in GenBank using the Gene names from Table 1. Proteins that are substantially similar to these and their corresponding gene sequences can be readily identified using the blast utilities available through GenBank.

TABLE 1
Immuno Reactive Proteins Identified by this Serological Screen

| | | | | | |
|---|---|---|---|---|---|
| | | | | | TM |
| | | | | | Domain/Sig. |
| Gene Name | Antigen | PI | Mol. Wt. | Description | Peptide |

Reactive in Immunized Mice, Humans & Macaques

| | | | | | |
|---|---|---|---|---|---|
| VACWR129 | A10L | 6.33 | 102,283 | major core protein | No/No |
| VACWR130 | A11R | 4.81 | 36,134 | hypothetical protein | Yes/No |
| VACWR132 | A13L | 9.96 | 7,696 | structural protein | Yes/Yes |
| VACWR156 | A33R | 5.3 | 20,506 | EEV glycoprotein | Yes/Yes |
| VACWR181 | A56R | 4.05 | 34,778 | hemagglutinin | Yes/Yes |
| VACWR187 | B5R | 4.54 | 35,108 | plaque-size/host range protein | Yes/Yes |
| VACWR113 | D8L | 9.55 | 35,326 | cell surface-binding protein | Yes/No |
| VACWR118 | D13L | 5.10 | 61,890 | rifampicin resistance protein | No/No |
| VACWR052 | F13L | 6.98 | 41,823 | major envelope protein | No/No |
| VACWR101 | H3L | 6.43 | 37,458 | IMV membrane protein | Yes/No |
| VACWR103 | H5R | 7.55 | 22,270 | late transcription factor | No/No |

Reactive in Immunized Humans & Macaques

| | | | | | |
|---|---|---|---|---|---|
| VACWR146/149* | A26L | 9.40 | 37,319 | A-type inclusion protein | No/No |

Reactive in Immunized Humans & Mice

| | | | | | |
|---|---|---|---|---|---|
| VACWR150 | A27L | 5.14 | 12,616 | cell fusion protein | No/No |
| VACWR059 | E3L | 5.04 | 21,504 | IFN resistance protein | No/No |
| VACWR091 | L4R | 6.13 | 28,460 | DNA-binding core protein | No/No |

Reactive in Immunized Mice & Macaques

| | | | | | |
|---|---|---|---|---|---|
| VACWR105 | H7R | 7.27 | 16,912 | hypothetical protein | No/No |

Reactive in Immunized Macaques Only

| | | | | | |
|---|---|---|---|---|---|
| VACWR137 | A17L | 4.28 | 22,999 | IMV membrane protein | Yes/Yes |

Reactive in Mice Only

| | | | | | |
|---|---|---|---|---|---|
| VACWR122 | A3L | 6.75 | 72,624 | major core protein | No/No |
| VACWR123 | A4L | 4.68 | 30,846 | Memb. Associated core | No/No |
| | | | | protein | |
| VACWR116 | D11L | 9.13 | 72,366 | DNA helicase | No/No |
| VACWR104 | H6R | 10.30 | 36,665 | topisomerase | No/No |
| VACWR033 | K2L | 9.73 | 42,299 | serine protease inhibitor | No/Yes |
| VACWR028 | N1L | 4.41 | 13,961 | Hypothetical proteins | No/No |

Reactive in Naïve (Non-immunized) Humans

| | | | | | |
|---|---|---|---|---|---|
| VACWR166 | A4IL | 4.90 | 25,092 | Secreted glycoprotein | No/Yes |
| VACWR173 | A47L | 10.29 | 28,334 | hypothetical protein | No/No |
| VACWR184 | B2R | 6.84 | 24,628 | hypothetical protein | No/No |
| VACWR115 | D10R | 8.12 | 28,934 | NTP phosphoydrolase | No/Yes |
| VACWR057 | E1L | 8.71 | 55,580 | poly(A) polymerase (VP55) | No/No |
| VACWR041 | F2L | 8.64 | 16,264 | dUTP pyrophosphatase | No/No |
| VACWR048 | F9L | 6.72 | 23,792 | Thiroedoxin substrate | Yes/Yes |
| VACWR082 | G5R | 4.93 | 49,872 | Core/assembly protein | No/No |
| VACWR085 | G7L | 7.72 | 41,920 | Structural/core protein | No/No |
| VACWR105 | H7R | 7.27 | 16,912 | hypothetical protein | No/No |
| VACWR070 | I1L | 9.05 | 35,841 | Telomere binding protein | No/No |
| VACWR092 | L5R | 10.32 | 15,044 | Myristylated protein | Yes/No |
| VACWR069 | O2L | 5.27 | 12,355 | glutaredoxin | No/No |

Reactive in Immunized Mice, Humans & Macaques

| | | | | | |
|---|---|---|---|---|---|
| VACWR129 | A10L | 6.33 | 102,283 | major core protein | No/No |
| VACWR130 | A11R | 4.81 | 36,134 | hypothetical protein | Yes/No |
| VACWR132 | A13L | 9.96 | 7,696 | structural protein | Yes/Yes |
| VACWR156 | A33R | 5.3 | 20,506 | EEV glycoprotein | Yes/Yes |
| VACWR181 | A56R | 4.05 | 34,778 | hemagglutinin | Yes/Yes |
| VACWR113 | D8L | 9.55 | 35,326 | cell surface-binding protein | Yes/No |
| VACWR118 | D13L | 5.10 | 61,890 | rifampicin resistance protein | No/No |
| VACWR052 | F13L | 6.98 | 41,823 | major envelope protein | No/No |
| VACWR101 | H3L | 6.43 | 37,458 | IMV membrane protein | Yes/No |
| VACWR103 | H5R | 7.55 | 22,270 | late transcription factor | No/No |

Reactive in Immunized Humans & Macaques

| | | | | | |
|---|---|---|---|---|---|
| VACWR146/149* | A26L | 9.40 | 37,319 | A-type inclusion protein | No/No |

Reactive in Immunized Humans & Mice

| | | | | | |
|---|---|---|---|---|---|
| VACWR150 | A27L | 5.14 | 12,616 | cell fusion protein | No/No |
| VACWR091 | L4R | 6.13 | 28,460 | DNA-binding core protein | No/No |

Reactive in Immunized Mice & Macaques

| | | | | | |
|---|---|---|---|---|---|
| VACWR187 | B5R | 4.54 | 35,108 | plaque-size/host range protein | Yes/Yes |
| VACWR105 | H7R | 7.27 | 16,912 | hypothetical protein | No/No |

Reactive in Immunized Macaques Only

| | | | | | |
|---|---|---|---|---|---|
| VACWR137 | A17L | 4.28 | 22,999 | IMV membrane proteins | Yes/Yes |

Reactive in Immunized Mice Only

| | | | | | |
|---|---|---|---|---|---|
| VACWR122 | A3L | 6.75 | 72,624 | major core protein | No/No |
| VACWR123 | A4L | 4.68 | 30,846 | Memb, associated core protein | No/No |
| VACWR116 | D11L | 9.13 | 72,366 | DNA helicase | No/No |
| VACWR059 | E3L | 5.04 | 21,504 | Adenosine deaminase | No/No |
| VACWR104 | H6R | 10.30 | 36,665 | topisomerase | No/No |
| VACWR033 | K2L | 9.73 | 42,299 | serine protese inhibitor | No/Yes |
| VACWR028 | N1L | 4.41 | 13,961 | Hypothetical prtoeins | No/No |

The proteins eliciting very strong seropositive reactions with VIG include A14L, A27L, H5R, D8R, D13L, DBL, H3L and F13L. Those proteins having moderate immunoreactivity were identified as A10L, A11R, L1R, B5R, A17L, 115L, F5L, A34L, A36R, A56R, and A13L. An additional protein giving a very strong seropositive response with VIG has also been identified; it is referred to as VACWR148, and has no close ortholog in the Copenhagen strain but is homologous to a protein named A29L in variola major. This protein has not previously been identified as antigenic and is referred to as an ATI locus protein herein.

By way of example only and without limiting the scope of proteins or DNA sequences encompassed by the invention, some of the closest orthologs for some of the immunoactive proteins identified by the present method include:

VACWR101 (VACV-COP H3L) Additional Orthologs:
VACV-MVA:MVA093L
RPXV-UTR:RPXV-UTR_090
VACV-AMVA:AMVA095
CPXV-GRI:J3L
VACV-TAN:Tan-TH3L
VARV-GAR: J3L
VARV-BSH:I3L
VARV-IND:I3L CMLV—
CMS:98L

VACWR118 (VACV-COP D13L) Additional Orthologs: VACV—
MVA:MVA110L
VACV-TAN: an-TD15L VACV—
AMVA:AMVA112
CPXV-GRI:E13L
RPXV-UTR:RPXV-UTR 107
VARV-BSH:N3L
VARV-IND: N3 L
CMLV-CMS:115L CMLV—
M96: CMLV116

VACWR 113 (VACV-COP D8L) Additional Orthologs: RPXV—
UTR:RPXV-UTR_102
VACV-MVA:MVA105L
VACV-AMVA: AMVA107
VACV-TAN:Tan-TD8L
VARV-IND:F8L
VARV-BSH:F8L
VARV-GAR:F8L
ECTV-NAV:EV-N-114
ECTV-MOS:EVM097

VACWR052 (VACV-COP F13L) Additional Orthologs: VACV—
TAN: an-TF13L
ECTV-NAV:EV-N-53
ECTV-MOS:EVM036
CPXV-GRI: G13L
RPXV-UTR:RPXV-UTR 041
VACV-AMVA:AMVA045
VACV-MVA:MVA043L
CPXV-BR:V061
VARV-GAR:E13L

VACWR103 (VACV-COP H5R) Additional Orthologs: RPXV—
UTR:RPXV-UTR_092
VACV-TAN:Tan-TH6R
VACV-AMVA:AMVA097
VACV-MVA:MVA095R
CPXV-GRI: J5R
MPXV-ZRE:H5R
VARV-BSH:I5R
CPXV-BR:V114
VARV-GAR: J5R

VACWR187 (VACV-COP B5R) Additional Orthologs: RPXV—
UTR:RPXV-UTR 167
VACV-TAN:Tan-TB5R
VACV-MVA:MVA173R
VACV-AMVA:AMVA173
CPXV-GRI:B4R
MPXV-ZRE:B6R
ECTV-MOS:EVM155
ECTV-NAV:EV-N-182
VARV-GAR:H7R

VACWR149 +VACWR146 (VACV-COP A26L) Additional Orthologs: RPXV—
UTR:RPXV-UTR 134
VACV-MVA:MVA137L
VACV-AMVA:AMVA139
CPXV-GRI: A27L
VACV-TAN:an-TA35L
MPXV-ZRE: A28L
CMLV-M96:CMLV145
CMLV-CMS:143L
CPXV-BR: V161

VACWR129 (VACV-COP A10L) Additional Orthologs: VACV—
MVA:MVA121L
VACV-AMVA:AMVA123
RPXV-UTR:RPXV-UTR 118
CPXV-GRI:A11L
VACV-TAN:an-TA11L
CMLV-M96:CMLV127
CMLV-CMS:126L
VARV-GAR:A11L
VARV-BSH: A11L

VACWR130 (VACV-COP Al 1R) Additional Orthologs: VACV—
AMVA:AMVA124
VACV-MVA:MVA122R CPXV—
BR:V143
CPXV-GRI: A12R
MPXV-ZRE: A12R
RPXV-UTR:RPXV-UTR 119
VACV-TAN:an-TA12R
ECTV-NAV:EV-N-131
ECTV-MOS:EVM114

VACWR181 (VACV-COP A56R) Additional Orthologs: VACV—
AMVA:AMVA167
VACV-MVA:MVA165R
VACV-TAN:an-TA66R
CPXV-GRI: A5 8R
MPXV-ZRE:B2R
CMLV-CMS: 173R
VARV-GAR:K9R
CMLV-M96: CMLV176
VARV-BSH:J7R

VACWR091 (VACV-COP L4R) Additional Orthologs:
VACV-MVA:MVA083R
RPXV-UTR:RPXV-UTR_080
VACV-AMVA: AMVA085
CPXV-BR:V102
CPXV-GRI:N4R
VACV-TAN:Tan-TL4R
VARV-IND:M4R
CMLV-M96:CMLV089
VARV-BSH:M4R
CMLV-CMS: 88R

VACWR156 (VACV-COP A33R) Additional Orthologs:
RPXV-UTR:RPXV-UTR_141
CPXV-GRI: A34R
VACV-TAN:R(TA43R)
VACV-MVA:MVA144R
VACV-AMVA:AMVA146
CMLV-M96:CMLV152
CMLV-CMS:150R
CPXV-BR:V168
MPXV-ZRE:A35R

Abbreviations used to describe these orthologs:
VACV-Cop=vaccinia virus strain Copenhagen
VACV MVA=vaccinia virus strain modified virus ankra
VACV-AMVA=Vaccinia virus strain Acambis 3000 MVA
VACVWR=vaccinia virus strain Western Reserve
VACV-TAN=Vaccinia virus strain Tian Tan
CPXV-GRI=cowpox strain GRI-90
RPV-UTR=Rabbitpox virus strain Utrecht
VARV-GAR=variola minor virus strain Garcia
VARV-BSH=variola major virus strain Bangladesh
VARV-IND=variola major virus strain India
CMLV-CMS=Camelpox virus strain CMS
CMLV-M96=Camelpox virus strain M96
ECTV-NAV=Ectromelia virus strain Naval (unpublished)
ECTV-MOS=Ectromelia virus Moscow strain
CPXV-BR=Cowpox virus strain Brighton Red
MPXV-ZRE=Monkeypox virus strain Zaire-96-1-16

Based on the foregoing, a suitable immunologic composition would comprise at least three proteins selected from the group of vaccinia proteins identified herein as antigenic, which group includes ATI locus proteins, A10L, A11R, A13L, A33R, A56R, B5R, D8L, D13L, F13L, H3L, H5R, A26L, A27L, E3L, L4R, H7R, A17L, A3L, A4L, D11L, H6R, K2L, N1L, A41L, A47L, B2R, D10R, E1L, F2L, F9L, GSR, G7L, H7R, I1L, L5R, and O2L. A second immunologic composition for the present invention comprises at least three proteins selected from those active in at least one immunized mammalian species tested, which proteins include ATI locus proteins, A10L, A11R, A13L, A33R, A56R, B5R, D8L, D13L, F13L, H3L, H5R, A26L, A27L, E3L, L4R, H7R, A17L, A3L, A4L, D11L, H6R, K2L, and N1L. A third immunologic composition within the present invention comprises at least three proteins selected from the group which are active in immunized humans, which group comprises ATI locus proteins, A10L, A11R, A13L, A33R, A56R, B5R, D8L, D13L, F13L, H3L, H5R, A26L, A27L, E3L, and L4R.

Other immunologic compositions within the present invention are those which comprise at least three proteins that were found by the present method to be reactive in immunized humans, mice and macaques (all three species), which group comprises A10L, A11R, A13L, A33R, A56R, B5R, D8L, D13L, F13L, H3L, and HSR. Another immunologic composition within the present invention comprises at least one protein selected from the group of antigens most consistently recognized by various immunized individuals, which group includes ATI locus proteins, A10L, A13L, H3L, D13L, A11R, and A17R. And based on an overall impression of the strength and consistency of responses, the types of proteins, and similar considerations, another preferred immunologic composition within the present invention comprises at least two, or more preferably at least three, of the following vaccinia proteins: ATI locus proteins, A10L, A13L, A26L, A56R, D8L, D13L, F13L, HSR, and H3L.

Preferred compositions within the present invention include those comprising at least two proteins selected from the group consisting of ATI locus proteins, A10L, D13L, and H3L. Other preferred immunologic compositions comprise one of the consistently immunoactive proteins or peptides or substantially homologous forms or immunoactive fragments thereof selected from the group consisting of A10L, D13L, H3L, and ATI locus proteins in combination with an additional vaccinia antigen. Thus, for example, particularly preferred combinations would include those which combine H3L (or its substantial homologs or immunoactive fragments) with an additional immunogenic vaccinia protein. Another such combination would comprise a protein encoded by the ATI locus or a substantial homolog or immunoactive fragment thereof with an additional immunogenic vaccinia protein. Yet another embodiment comprises at least one protein selected from the group of novel antigens comprising A11R, A23L, A56R, and HSR, or one of these antigens in combination with at least one other antigenic vaccinia protein.

For each of the foregoing vaccine compositions, the invention also includes the corresponding DNA vaccines. Thus for each group of proteins set forth herein, a vaccine composition comprising the group of genes corresponding to the specified proteins is also within the scope of the invention as are the corresponding combinations of such genes with the corresponding vaccinia antigenic protein genes.

Thus the methodology identifies novel immunologically reactive antigens, not all of which would be identified by conventional predictive approaches. Data obtained with the arrays are in agreement with immunoblots we have reported previously, Crotty, S., et al., J. Immunol. (2003) 171:4969-4973, which is incorporated herein in its entirety by reference. Notably in vaccinated humans, we see strong anamnestic responses to a subset of dominant antigens after boosting many years after the primary immunization, notably to the H3L, D13L and A10L proteins.

### EXAMPLE 7

### Comparison of Protein Expression Using Plasmids Isolated From Single Colony/Clone or from Mixture of Transformation Culture

Twenty-eight (28) target genes ranging from 300 bp to 2000 bp in size from F. tularensis were selected and amplified by PCR using primers that contain 20 bp gene-specific sequence and 30 bp adaptor sequence homologous to corresponding ends of linear pIX expression vector (conferring T7 promoter and N-terminal poly-histidine fusion), as described above.

Twenty-five (25) ng of PCR product was pre-mixed with the same amount oflinear piX prep. The DNA mixture was transformed into 50 μl chemically competent E. coli DH5a cells, left on ice for 30 minutes, heat-shocked for 45 seconds at 45° C., and mixed with 500 μl of SOC media followed by incubation at 37° C. After 1 hour, 500 μl of LB media containing Kanamycin (50 μg/ml) was added followed by continuous incubation at 37° C. with shaking for >14-24 hours.

For single clone procedure, 50 μl of the culture was then plated onto a LB agar plates with Kanamycin selection (25 μg/ml) and incubated again at 37° C. for 12-14 hours. A single colony was then picked and cultured again overnight using the same media followed by DNA isolation using Qiagen miniprep kit.

Alternatively, plasmid DNA was isolated directly from the overnight transformation mixture in the first step, above.

The plasmid DNA (5 μl) from steps 2 and 3 was added to 20 μl Roche RTS 100 cell-free transcription/translation mix and incubated at 30° C. for 4 hours. 0.5 μl of the expression mixture was spotted onto a nitrocellulose membrane followed by standard Western blot detection of the expressed protein using anti-poly-histidine tag monoclonal antibody.

### Table 2

### Protein Expression From Single Clone and Transformation Mixture (Results Showing Difference Between the Two Methods are Highlighted in Red Color)

Gene Name Expression of his-tag fusion
   Single colony Mixed culture
#1788
#884
#1532
#558
#267
#226
#1148
#401
#316
#513
#617
#619
#397
#1894
Gene Name Expression of his-tag fusion
   Single colony Mixed culture
#968
#257
#344
#1101
#570
#318
#352
#1531
#1056
#1167
#661
#2009
#1437
#1819

Single clone: 18 out 28 samples showed expression of the target gene. 10 samples did not give rise to any detectable level of protein expression.

Transformation mixture: 23 out of 28 samples showed expression. Five out of 10 negative samples from single clone protocol showed expression indicating plasmids from the single colonies may contain mutation(s) that prevented encoded protein from being expressed.

### EXAMPLE 8

### H3L Epitope Scan

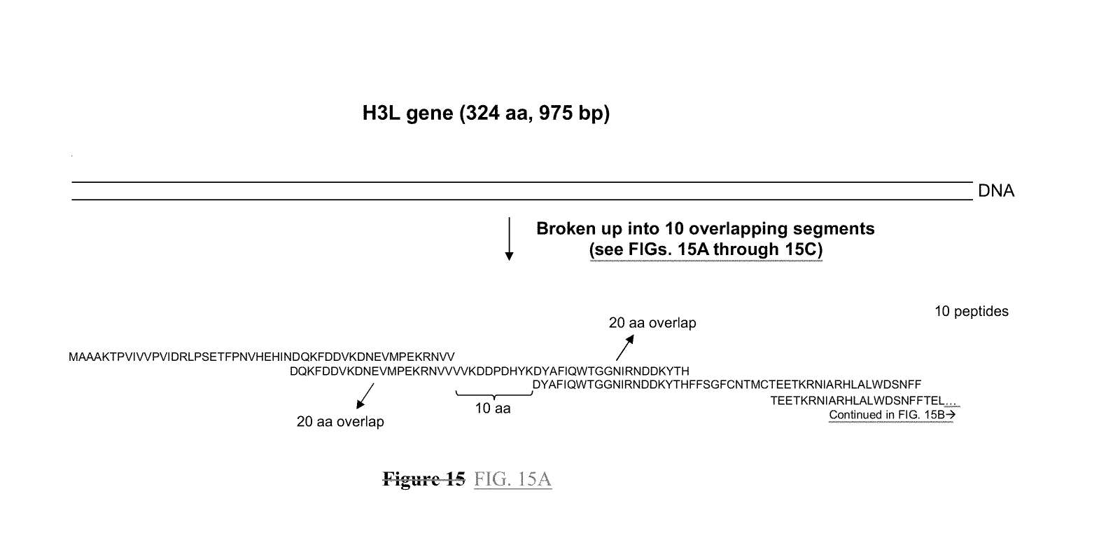

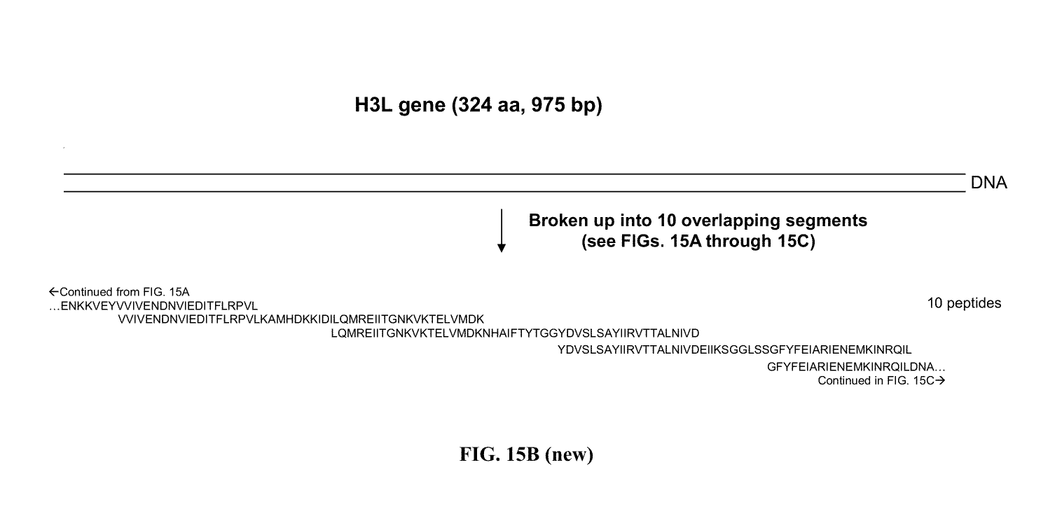

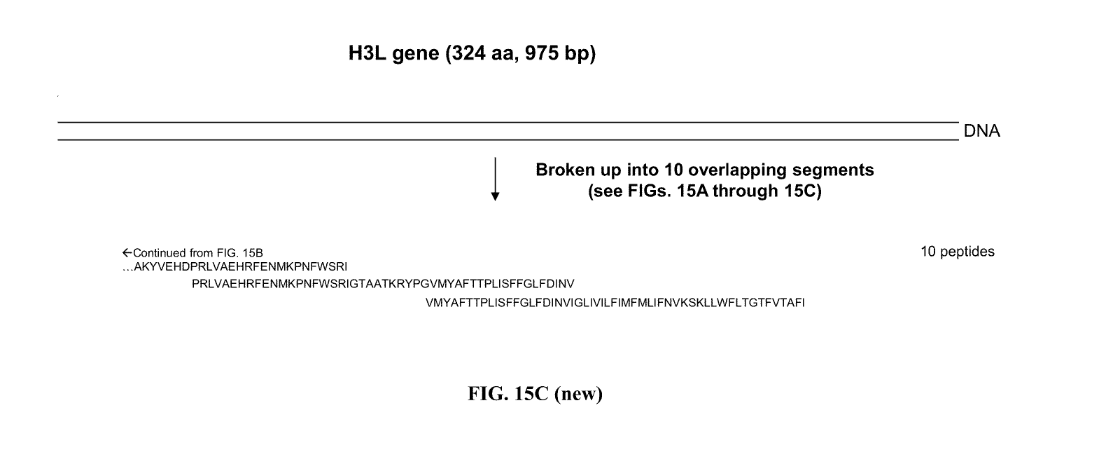

The vaccinia envelope protein H3L was divided into 10 overlapping segments of 50 amino acids as shown in FIGS. 15A-15C. For each segment, forward and reverse primers, each 53 bp long, were designed, as are shown in Table 3. The primer sequences include 33 bp of DNA complementary to the ends of the pXi (source) vector when linearized at the BamH1 site, and 20 bp of DNA complementary to the end of the specific segments.

To PCR amplify each segment, vaccinia genomic DNA was mixed with 10 μM of the specific forward and reverse primers, water and Eppendorf HotMaster Mix to a final volume of 50 μl. For 30 cycles, denaturation took place at 94° C. for 30 sec, followed by annealing at 50° C. for 30 sec and extension at 68° C. for 30 sec. After PCR, the products were run on a 1% agarose gel to assess the success of amplification. One gel showed enough products of segments 1, 2, and 6, a scanned gel showed enough of 3, 4, 8, and 10, and a third gel showed enough of 9. None of the PCR reactions successfully amplified segments 5 and 7. Therefore, instead of amplifying these two 150 bp segments, forward and reverse primers of 4 and 6 respectively were used to amplify 5, and forward and reverse primers of 6 and 8 were used to amplify 7. The amplification of these 450 bp sequences was successful.

After PCR amplification and cleanup of the PCR product using Qiagen PCR Purification Kit, the segments were cloned using recombination cloning. 40 ng of linearized pXi vector was mixed with 10 ng of cleaned up PCR product and to this mixture, 10 μl of DH5 alpha E. coli competent cells was added. The mixture was then placed on ice for 45 minutes, heat shocked at 42° C. for 1 minute and then moved back to the ice for another minute. The mixture was removed and 200 μl of SOC media was added to each tube and the mixture incubated in a 37° C. water bath for 1 hour. The transformation mixture was mixed with 3 mL of LB+Kanamycin and incubated overnight at 37° C.

Plasmid DNA was isolated from the transformation mixture using miniprep. Gels were run to determine if the plasmid had the insert. As a control, circular pXi vector was run. The results show that plasmids designed to contain segments 1, 2, 3, 6, 8, 9, and 10 had insert.

TABLE 3
H3L Primers

| Fragment | DNA sequence | FP (5′-3′) | RP (5′-3′) |
|---|---|---|---|
| (1) | ATGGCGGCGGCGAAAACTCCTGTTATTGTTGTG | CATATCGACGACGACGAC | CATATCGACGACGACGAC |
| | CCAGTTATTGATAGACTTCCATCAGAAACATTT | AAGCATATGCTCGAGATG | AAGCATATGCTCGAGATG |
| | CCTAATGTTCATGAGCATATTAATGATCAGAAG | GCGGCGGCGAAAACTCC | GCGGCGGCGAAAACTCC |
| | TTCGATGATGTAAAGGACAACGAAGTTATGCCA | | |
| | GAAAAAAGAAATGTTGTG | | |
| (2) | GATCAGAAGTTCGATGATGTAAAGGACAACGAA | CATACTCACGACGACGAC | ATCTTAAGCGTAATCCGG |
| | GTTATGCCAGAAAAAAGAAATGTTGTGGTAGTC | AAGCATATGCTCGAGGAT | AACATCGTATGGGTAGGT |
| | AAGGATGATCCAGATCATTACAAGGATTATGCG | CAGAAGTTCGATGATGT | GAGTATACTTGTCATCAT |
| | TTTATACAGTGGACTGGAGGAAACATTAGAAAT | | |
| | GATGACAAGTATACTCAC | | |
| (3) | GATTATGCGTTTATACAGTGGACTGGAGGAAAC | CATATCGACGACGACGAC | ATCTTAAGCGTAATCCGG |
| | ATTAGAAATGATGACAAGTATACTCACTTCTTT | AAGCATATGCTCGAGGAT | AACATCGTATGGGTAGAA |
| | TCAGGGTTTTGTAACACTATGTGTACAGAGGAA | TATGCGTTTATACAGTG | AAAAATTAGAATAGAAAC |
| | ACGAAAAGAAATATCGCTAGACATTTAGCCCTA | | G |
| | TGGGATTCTAATTTTTTT | | |
| (4)_ | ACAGAGGAAACGAAAAGAAATATCGCTAGACAT | CATATCGACGACGACGAC | ATCTTAAGCGTAATCCGG |
| | TTAGCCCTATGGGATTCTAATTTTTTTACCGAG | CTCGAGACAGAGGAAACG | AACATCGTATGGGTAGCA |
| | TTAGAAAATAAAAAGGTAGAATATGTAGTTATT | AAAAGAAA | AGCCATTACAAGCTCGG |
| | GTAGAAAACGATAACGTTATTGAGGATATTACG | | |
| | TTTCTTCGTCCCGTCTTG | | |
| (5) | GTAGTTATTGTAGAAAACGATAACGTTATTGAG | CATATCGACGACGACGAC | ATCTTAAGCGTAATCCGG |
| | GATATTACGGCAATGCATGACAAAAAATAGATA | AAGCATATGCTCGAGGTA | AACATCGTATGGGTAGTT |
| | TCCTACAGATGAGAGAAATTATTACAGGCAATA | GTTATTGTAGAAAACGA | TGTCCATTACAAGCTCGG |
| | AAGTTAAAACCGAGCTTGTAATGGACAAA | | |
| (6) | CTACAGATGAGAGAAATTATTACAGGCAATAAA | CATATCGACGACGACGAC | ATCTTAAGCGTAATCCGG |
| | GTTAAAACCGAGCTTGTAATGGACAAAAATCAT | AAGCATATGCTCGAGCTA | AACATCGTATGGGTAGAT |
| | GCCATATTCACATATACAGGAGGGTATGATGTT | CAGATGAGAGAAATTAT | CTACGATGTTCAGCGCCG |
| | AGCTTATCAGCCTATATTATTAGAGTTACTACG | | |
| | GCGCTGAACATCGTAGAT | | |
| (7) | TATGATGTTAGCTTATCAGCCTATATTATTAGA | CATATCGACGACGACGAC | ATCTTAAGCGTAATCCGG |
| | GTTACTACGGCGCTGAACATCGTAGATGAAATT | AAGCATATGCTCGAGTAT | AACATCGTATGGGTAGCA |
| | ATAAAGTCTGGAGGTCTATCATCGGGATTTTAT | GATGTTAGCTTATCAGC | GTATCTGCCTATTGATCT |
| | TTTGAAATAGCCAGAATTGAAAACGAAATGAAG | | |
| | ATCAATAGGCAGATACTG | | |
| (8) | GGATTTTATTTTGAAATAGCCAGAATTGAAAAC | CATATCGACGACGACGAC | ATCTTAAGCGTAATCCGG |
| | GAAATGAAGATCAATAGGCAGATACTGGATAAT | AAGCATATGCTCGAGGGA | AACATCGTATGGGTAGTA |
| | GCCGCCAAATATGTAGAACACGATCCCCGACTT | TTTTATTTTGAAATAGC | TTCTAGACCAAAAATTCG |
| | GTTGCAGAACACCGTTTCGAAAACATGAAACCG | | |
| | AATTTTTGGTCTAGAATA | | |
| (9) | CCCCGACTTGTTGCAGAACACCGTTTCGAAAAC | CATATCGACGACGACGAC | ATCTTAAGCGTAATCCGG |
| | ATGAAACCGAATTTTTGGTCTAGAATAGGAACG | AAGCATATGCTCGAGCCC | AACATCGTATGGGTAGAA |
| | GCAGCTACTAAACGTTATCCAGGAGTTATGTAC | CGACTTGTTGCAGAACA | CATTAATATCAAACAATC |
| | GCGTTTACTACTCCACTGATTTCATTTTTTGGA | | |
| | TTGTTTGATATTAATGTT | | |
| (10) | GTTATGTACGCGTTTACTACTCCACTGATTTCA | CATATCGACGACGACGAC | ATCTTAAGCGTAATCCGG |
| | TTTTTTGGATTGTTTGATATTAATGTTATAGGT | AAGCATATGCTCGAGGTT | AACATCGTATGGGTAGTT |
| | TTGATTGTAATTTTGTTTATTATGTTTATGCTC | ATGTACGCCTTTACTAC | AGATAAATGCGGTAACGA |
| | ATCTTTAACGTTAAATCTAAACTGTTATGGTTC | | |
| | CTTACAGGAACATTCGTTACCGCATTTATCTAA | | |

### EXAMPLE 9

### Detection of T-Cell Activation Using Proteins Immobilized on Beads

Using the methods described above, substantially all of the proteome of the organism in question (e.g. vaccinia) is cloned using a T7 vector (pTX7) and the proteins are expressed using a cell-free in vitro system. The adapter used to insert each protein into the vector includes a poly-His tag so the expressed proteins can be captured onto 1 μm nickel-coated beads that have been previously equilibrated in a loading buffer (300 mM NaCl, 50 mM sodium phosphate 10 mM imidazole, pH 8.0). The nickel-coated beads may be of various sizes but are advantageously smaller than the APC cells, which are typically about 10-20 microns in diameter; nickel-coated beads that are 1-3 microns in size are available and sufficient for this purpose. The protein-coated beads are then washed 5 times in washing buffer (as above except with 20 mM imidazole), twice in tissue culture medium, and then resuspended in serum free medium to the original 12.5 μl volume. These beads are incubated with antigen presenting cells prior to combining with T cells in 96 well assay format.

Responder T cells are obtained from mice immunized with the pathogen (e.g., 2×10⁵ pfu vaccinia administered intraperitoneally) or with individual recombinant proteins in adjuvant administered i.p. or subcutaneously at the base of the tail, or from the peripheral blood of infected/immunized human donors. In the case of mice, spleens or draining lymph nodes are removed 7-10d after immunization. Antigen-coated beads (usually 1-5 μl per well) are then added to murine splenocytes or human peripheral blood mononuclear cells (PBMC; 5×10⁵ cells/well) in Multiscreen 96 well plates (Millipore MAHAS45) precoated with (from Pharmingen) and blocked for 1 h in tissue culture medium containing 10% fetal calf serum (FCS) (murine assays) or 5% human AB serum (human assays). The anti-mouse or human IFN-γ may be fixed into the well on a nitrocellulose substrate, for example; in that case, the treatment with serum serves to block any unoccupied sites on the nitrocellulose that could otherwise bind the capture antibody and interfere with the ELISPOT assay used to detect interferon or other cytokines formed. The IFN-γ antibodies capture any IFN-γ produced when the T-cells (splenocytes or PBMC) are stimulated by a recognized antigen. Thus after rinsing away unbound materials, any IFN-γ formed remains bound to the IFN-γ capture antibodies and is detected by addition of a second antibody capable of binding to the bound IFN-γ. This second antibody is labeled for easy visualization.

The medium used may be Iscove's Modified Dulbecco's Medium (IMDM) with Penicillin/Streptomycin/Glutamine and supplemented with 10-SOJ.Lg/ml polymyxin B to inhibit any contaminating LPS. For murine T cell assays, the medium is also supplemented with 2-mercaptoethanol to a final concentration of 5×10⁻⁵ M. Positive control antigens for human assays may include tetanus toxoid, adsorbed onto alum (Colorado Serum Co) used at 1/160 and in TB-vaccinated donors, purified protein derivative (Tubersol from Aventis Pasteur). Mitogens that can be used to confirm assay and cell viability include Concanavalin-A for mouse cells and phytohemagglutinin for human cells, both used at 1 μg/ml. Antibodies for IFN-γ detection by ELISPOT are matched pairs from Pharmingen.

After 18 to 20 h of co-cultivation, captured interferon is detected with biotinylated anti-IFN-γ detection antibody (Pharmingen) and visualized with streptavidin-alkaline phosphatase followed by nitro-BT developer. Supernatants of human and murine cultures are also taken at 6 h, 12 h, 24 h and 48 h and subjected to multiplex cytokine analysis (using custom 10-plex kits from Linco Research Inc) for Thl (IFN-γ, TNF-α, and IL-12) Th2 (IL-4, IL-6, IL-10 and IL-13) and inflammatory cytokines (IL-1β, IL-2 and GM-CSF) and maybe analyzed simultaneously using a Luminex 100 machine. The presence of one or more of these cytokines demonstrates that the protein being tested elicits a cellular immune response, and allows one to identify those proteins or peptides useful for eliciting immunity.

### EXAMPLE 10

### Detection Off-Cell Activation Using Expression of Proteins in APCs

Substantially all of the proteome of the organism in question (e.g. vaccinia) is cloned into the CMV (gWIZ) vector. Plasmids are introduced in antigen presenting cells (APCs) using lipid delivery (by “Lipofection”, using special lipid reagents such as Lipofectin™ from Invitrogen, Cytofectene™ Transfection Reagent by Bio-Rad, or FuGENE 6™ Transfection Reagent by Roche Applied Science; see Feigner, et al., Proc. Nat'l. Acad. Sci. USA., November 1987 84(21), 7413-7, which is incorporated herein in its entirety by reference) after 1 day, to allow the proteins to be expressed prior to combining with T cells in 96 well assay format. Responder T cells are obtained from mice immunized with the pathogen (e.g., 2×10⁵ pfu vaccinia administered intraperitoneally) or with individual recombinant proteins in adjuvant administered i.p. or subcutaneously at the base of the tail, or from the peripheral blood of infected/immunized human donors. In the case of mice, spleens or draining lymph nodes are removed 7-10 days after immunization. Transfected antigen presenting cells are then added to murine splenocytes or human PBMC (5×10⁵ cells/well) in Multiscreen 96 well plates (Millipore MAHAS45) precoated with anti-mouse or human IFN-′Y (from Pharmingen) and blocked for 1 h in tissue culture medium containing 10% FCS (murine assays) or 5% human AB serum (human assays).

The medium used may be Iscove's Modified Dulbecco's Medium (IMDM) with Penicillin/Streptomycin/Glutamine and supplemented with 10-50 μg/ml polymyxin B to inhibit any contaminating LPS (lipopolysaccharides). For murine T cell assays, medium is also supplemented with 2-mercaptoethanol to a final concentration of 5×10⁵M. Positive control antigens for human assays may include tetanus toxoid, adsorbed onto alum (Colorado Serum Co) used at 1/160 and in TB-vaccinated donors, purified protein derivative (Tubersol from Aventis Pasteur). Mitogens to confirm assay and cell viability can include Concanavalin-A for mouse cells and phytohemagglutinin for human cells, each of which is used at 1 μg/ml. Antibodies for IFN-γ detection by ELISPOT are matched pairs from Pharmingen.

After 18 to 20 h of co-cultivation, captured interferon is detected with biotinylated anti-IFN-γ detection antibody (Pharmingen) and visualized with streptavidin-alkaline phosphatase followed by nitro-BT developer. Supernatants of human and murine cultures are also taken at 6 h, 12 h, 24 h and 48 h and subjected to multiplex cytokine analysis (using custom 10-plex kits from Linco Research Inc) for Thl (IFN-γ, TNF-α, and IL-12) Th2 (IL-4, IL-6, IL-10 and IL-13) and inflammatory cytokines (IL-1β, IL-2 and GM-CSF) and maybe analyzed simultaneously using a Luminex 100 machine. The presence of one or more of these cytokines demonstrates that the protein being tested elicits a cellular immune response, and allows one to identify those proteins or peptides useful for eliciting immunity.

### EXAMPLE 11

### Validation of the Antigen Identification Method Using Malaria (P. (alciparum)

A set of 218 P.falciparum (Pf) genes were selected for cloning, expression, and protein microarray chip printing. The genes were selected on the basis of subcellular localization (e.g., secreted proteins and other proteins found in cell culture supernatants), known immunogenicity in human and animal models of P. falciparum, and pattern of gene expression vis-a-vis Plasmodium growth state. Each fit into one of nine categories: i) Identified by bioinformatic criteria only (n=25); ii) Identified by laser capture microdissection of P. yoelii liver-stages, and identified in sporozoite proteome by MudPIT (n=16); iii) Pf orthologues of proteins identified by laser capture microdissection ofPy liver-stage but not found in sporozoite proteome (liver-stage specific; n=52); iv) Highly expressed in sporozoite proteome by MudPIT (n=10); v) Identified in sporozoite proteome by MudPIT and assayed for immune recognition by PBMCs from irradiated sporozoite (irr-spz) immunized volunteers (n=27); vi) Known and well characterized Pf antigens in clinical development (n-21); vii) Highly expressed in sporozoite stage as evidenced by gene transcript profiling of sporozoites by Affymetrix gene chips (n=53); viii) Identified in trophozoite and schizont-stage proteome by MudPIT (n=11); and ix) P. falciparum orthologues of P. yoelii antigens indicated to be protective in vivo (n=2). One additional gene of interest that was included, PFB0645c, does not fit into any of these categories.

PCR amplification was accomplished using P. falciparum genomic DNA template. Since many P. falciparum genes contain introns, primers were designed to span each exon. Large genes (and exons) greater than 3000 base pairs were amplified in segments with each segment overlapping by 150 nucleotides (i.e. 50 amino acids). Primer design covering the entire P. falciparum genome was done by Arlo Randall at the Institute of Genomics and Bioinformatics at UC Irvine and the primer database is accessible through a Web interface. The database contains 14,446 entities. Thus to amplify each independent exon and to amplify large genes in segments less than 3000 bp would require 14,446 primer pairs. However, about 40% of the ORFs encode short peptides less than 50 amino acids, so about 8000 primer pairs would be required to amplify each ORF greater than 150 nucleotides. This on-line database was used as the source of primer sequences for the following study.

A total of 266 ORFs derived from the 218 gene target set were amplified, cloned, and expressed using the expressions system previously described. Using a process that took 3 days to complete, 266 ORFs were PCR amplified from P. falciparum genomic DNA, the fragments were cloned into a T7 expression vector, expressed in a cell-free in vitro transcription/translation system and the expressed proteins were spotted onto microarray chips. The chips were probed with E. coli lysate treated sera from irradiated sporozoite immunized human volunteers, the slides were developed with Cy3 labeled anti-human antibody and read with a laser confocal microarray chip reader. The malaria immune individuals reacted against a subset of P. falciparum proteins, whereas naive individuals were not reactive. The proteins were printed onto microarray chips, and the chips were probed with sera from 11 donors who were naturally exposed to malaria in hyperendemic region of Kenya, or had been immunized with irradiated sporozoites. Naive donors lacked reactivity against the complete set of expressed proteins printed on the chip (FIG. 6), but sera from immunized individuals reacted against a subset of proteins on the chip. A summary of these results is shown in Table 4. The “gene locus” codes in Table 4 correspond to the “locus tag” codes utilized in the GenBank database, available online at the web address www.ncbi.nlm.nih.gov/gquery/gquery.fcgi. Thus the codes can readily be used to obtain both the DNA sequence and the peptide sequence for each of the proteins in the Table.

There were 9 strongly reactive proteins identified from this analysis. Seven out of the nine highly reactive proteins are known, well characterized Pf blood-stage antigens, many of which are under clinical development and evaluation (LSA3, MSP4, EBA175, RESA). Interestingly, PF10_0356, Liver Stage Antigen 1, is a liver-stage specific antigen; it is NOT expressed in the sporozoite or blood-stages of the organism, only in the liver stage. So the fact that 6 of 11 sera recognized this antigen demonstrates that the proteome arrays have the capacity to identify more than just the blood stage antigens. Also, PFD031Ow is SHEBA/Pfs16, a sexual stage antigen under clinical development as a vaccine antigen candidate. One of the most strongly reactive antigens, PFE1590w has not been previously recognized as a potential vaccine antigen candidate.

TABLE 4
Serum Reactivity in Malaria Immune Subjects.

| #of | | |
|---|---|---|
| Responders | Gene Locus | Protein ID |
| 11 | PFB0300c | merozoite surface protein 2 precursor |
| | | (MSP2) |
| 11 | PFB0915w* | liver stage antigen 3 (LSA3) |
| 10 | PFB0310c* | merozoite surface protein 4 (MSP4) |
| 9 | PFE1590w | early transcribed membrane protein |
| 8 | PFD0310w | sexual stage-specific protein precursor |
| | | (SHEBA/Pfs16) |
| 6 | PF07_0128 | erythrocyte binding antigen (EBA175) |
| 6 | PF10_0343* | S-antigen |
| 6 | PF10_0356 | liver stage antigen, putative (LSA1) |
| 6 | PF11_0509* | ring-infected erythrocyte surface antigen |
| | | (RESA) |

*These genes included introns, and were expressed as two separate proteins, overlapping by 20 amino acids. At least one of the two proteins is antigenic.

By way of example only and without limiting the scope of proteins or DNA sequences encompassed by the invention, some of the closest orthologs for some of the immunoactive proteins identified by the present method, some of which are not in Table 4, include:

PFB0310c:

P. yoelii: PY05967 (MSP4/5 related)

P. yoelii: PY07543 (MSP 4/5)

PFE1590w:

P. yoelii: PY02667 (integral membrane protein)

PFB07 0128:

P. falciparum: Chr. 13, MAL13P1.60 (erythrocyte binding antigen 140)

P. falciparum Chr. 1, PFA0125c (Ebl-11ike protein, putative)

P. falciparum Chr. 1, PFA0065w (hypothetical protein)

P. falciparum Chr. 4, PFD1155w (erythrocyte binding antigen, putative)

P. yoelii PY04764 (duffy receptor, beta form precursor)

PF10 0343:

P. yoellii PY04926 (hypothetical protein)

PF11 0509:

| gene | species | description |
|---|---|---|
| MAL6P1.19 | P. falciparum | hypothetical protein |
| MAL7P1.174 | P. falciparum | hypothetical protein |
| MAL7P1.7 | P. falciparum | RESA-like protein |
| MAL8P1.2 | P. falciparum | hypothetical protein with DNAJ domain |
| PF10_0378 | P. falciparum | hypothetical protein |
| PF11_0037 | P. falciparum | hypothetical protein |
| PF11_0509 | P. falciparum | ring-infected erythrocyte surface |
| | | antigen, putative |
| PF11_0512 | P. falciparum | ring-infected erythrocyte surface |
| | | antigen 2, RESA-2-malaria parasite |
| | | (Plasmodium falciparum)-related |
| PF11_0513 | P. falciparum | hypothetical protein |
| PF14_0018 | P. falciparum | hypothetical protein |
| PF14_0732 | P. falciparum | hypothetical protein |
| PF14_0746 | P. falciparum | hypothetical protein |
| PFA0110w | P. falciparum | ring-infected erythrocyte surface |
| | | antigen precursor |
| PFB0080c | P. falciparum | hypothetical protein |
| PFB0085c | P. falciparum | hypothetical protein |
| PFB0920w | P. falciparum | hypothetical protein |
| PFD0095c | P. falciparum | hypothetical protein |
| PFD1170c | P. falciparum | hypothetical protein |
| PFD1180w | P. falciparum | Plasmodium falciparum trophozoite |
| | | antigen-like protein |
| PFE1600w | P. falciparum | hypothetical protein |
| PFE1605w | P. falciparum | protein with DNAJ domain |
| PFI0130c | P. falciparum | hypothetical protein |
| PFI1785w | P. falciparum | hypothetical protein |
| PFI1790w | P. falciparum | hypothetical protein |
| PFL0055c | P. falciparum | protein with DNAJ domain |
| | | (resa-like), putative |
| PFL2535w | P. falciparum | RESA-like protein, putative |
| PFL2540w | P. falciparum | hypothetical protein |

PF13 0197:

P. falciparum: CHR 13/MAL13P1.173/MSP7-like protein

P. falciparum: CHR 13/MAL13P1.174/MSP7-likeprotein

P.falciparum: CHR 13/PF13_0193/MSP7-like protein

P.falciparum: CHR 13/PF13_0196/MSP7-like protein

P.falciparum: CHR 13/PF13_0197/Merozoite Surface Protein 7 precursor,

MSP7

P. yoelii: PY02147/Meloidogyne incognita COL-1-related

PF14_0486:

P. yoelii PY05356 (elongation factor 2)

PF08_0054:

P.yoelii PY06158 (heat shock protein 70)

PF11_0344:

P. yoelii PY01581 (apical membrane antigen-1)

In a separate application of these methods, 300 genes from P. falciparum were expressed and displayed in a microarray using the methods described herein. The array was probed with serum from 12 subjects who contracted malaria at an early age and were thus immunized to it. Positive responses were observed in at least six of the twelve serum samples for each of the following gene products:

TABLE 4b
Serum Reactivity in Malaria Immune Subjects.

| Genes | | Positive |
|---|---|---|
| (Locus tag | | responses |
| used in GenBank) | Description from GenBank | (out of 12 sera) |
| PFB0915w | LSA-3-e2s1 | 12 |
| PFB0310c | MSP-4-e1 | 12 |
| PFB0300c | MSP-2 | 12 |
| PFB0305c | MSP-5-e1 | 12 |
| PFL2410w | hypothetical protein-e1 | 12 |
| PFC0210c | Circumsporozoite (CS) prot | 12 |
| PFD0310w | sex stg-spec prot prec a | 11 |
| PFD0310w | sex stg-spec prot prec b | 11 |
| PF13_0197 | MSP7 precursor | 11 |
| PF10_0138 | hypothetical prot-s1 | 11 |
| PFI1520w | hypothetical protein b | 11 |
| PFI1520w | hypothetical protein a | 11 |
| PF11_0344 | ap memb antigen 1 prec | 11 |
| PF13_0012 | hypothetical prot | 10 |
| PFD0310w | sex stg-spec prot prec | 10 |
| PF11_0358 | DNA-dir RNAP, B subunit-e1 | 10 |
| PF07_0029 | HSP86-e1 | 10 |
| PFL1605w | hypothetical prot-s2 | 10 |
| PFE1590w | early transc memb prot | 10 |
| MAL6P1.201 | leucyl-trna synthetase, | 10 |
| | cytoplasmic-s2 | |
| PFD0235c | hypothetical prot-e1 | 9 |
| PF13_0201 | spz surf prot 2 | 9 |
| PF13_0267 | hypothetical protein a | 9 |
| PF07_0128 | erythrocyte binding antigen-e1s2 | 9 |
| PF10_0343 | S-Antigen a | 9 |
| PF10_0343 | S-Antigen | 9 |
| PFI1520w | hypothetical protein | 8 |
| PFI0580c | Hypo Asn-rich prot w/N-term sig | 8 |
| | seq-e2 | |
| PF07_0020 | hypothetical prot-e1s2 | 8 |
| PFE0520c | topoisomerase I | 8 |
| MAL7P1.29 | hypothetical protein-e1s2 | 8 |
| PF10_0260 | hypothetical protein-e2s2 | 8 |
| PF11_0358 | DNA-dir RNAP, B subunit-e2s2 | 7 |
| MAL8P1.139 | hypothetical prot-e3 | 7 |
| PF13_0228 | PF01092 Rib prot S6e | 7 |
| PF10_0132 | phospholipase C-like-e1s2 | 7 |
| PFB0855c | hypothetical prot-e2 | 7 |
| PF10_0125 | hypothetical prot | 7 |
| PF13_0350 | SRP54-type prot, GTPase dam | 7 |
| PFD0665c-e2 | | 7 |
| MAL7P1.32 | hypothetical prot | 7 |
| PF07_0016 | hypothetical prot-s1 | 7 |
| PF10_0098a | | 6 |
| PF08_0056 | zinc finger protein-e2 | 6 |
| PFB0640c-e1s1 | | 6 |
| PF14_0230 | Rib prot fam L5-e2 | 6 |
| PF14_0315 | hypothetical prot-e2s1 | 6 |
| PF08_0088 | hypothetical prot | 6 |
| PFL0685w | hypothetical prot-e2 | 6 |
| MAL7P1.23 | hypothetical prot-e1s2 | 6 |
| PFE0060w | hypothetical prot-e2 | 6 |
| MAL8P1.23 | ubiquitin-prot ligase 1-s8 | 6 |
| PF07_0029 | HSP86-e2 | 6 |
| PF10_0356 | LSA-e2s2 | 6 |

### EXAMPLE 12

### Malaria Vaccines and Diagnostic Tests

From the data set obtained in Example 11, a cocktail of proteins or nucleic acids encoding proteins is selected for a vaccine composition. A malaria vaccine cocktail based on these results comprises at least three of the following genes or the corresponding peptides, and four or more, or five or more, or it may include all of these: PFB0300c, PFE1590w, PFB0915w, PFB0310c, PFB0310w, PF11_0509, and PF10_0343. This vaccine is administered using the excipients, compositions and methods disclosed herein to immunize a human subject at risk for malaria, provided the subject's immune system is not compromised.

Alternatively, a vaccine would comprise at least three of the nucleic acids or three of the proteins corresponding to the genes identified in Table 4b as ones expressing antigenic proteins. In a preferred embodiment, the vaccine would comprise more than three or more than four or at least six of these proteins or nucleic acids. Typically, the vaccine wouldcomprise at least three nucleic acids or proteins corresponding to the genes whose gene product gave a positive response in at least six of the tested sera, or in at least 8 of the tested sera; or in at least 9 of the tested sera; or in at least 10 of the tested sera; or in at least 11 of the tested sera. In some embodiments, the vaccine would comprise at least one component corresponding to one of the genes that elicited a positive response in 10 or more of the sera tested. In other embodiments, the vaccine would comprise at least two protein or nucleic acid components or at least three protein or nucleic acid components corresponding to genes that elicited a positive response in 10 or more of the 12 sera tested. In other embodiments the immunodomiant antigens would be used in a serological diagnostic test, such as ELISA, to unambiguously diagnose whether a person has be exposed or infected by P. falciparum.

### EXAMPLE 13

### Antigenic Proteins Identified in Francisella Tularensis

Following the methods described above using the proteins of Example 1D from F. tularensis, a number of antigenic proteins were identified that were reactive with serum from mice that were exposed to a non-infectious strain of Francisella or from mice that were exposed to the virulent Schu S4 strain. Data for those proteins is in Tables 5 and 6 below. The sequences for the proteins are available in the GenBank database, which is available online at the web address www.ncbi.nlm.nih.gov/gquery/gquery.fcgi. The gene code in the table corresponds to the locus tag for the gene and protein identified.

TABLE 5
Antigens detected with serum from mice exposed to non-infectious
strain.

| | | Mice exposed to non-infectious strain | | | |
|---|---|---|---|---|---|
| | | (each col. Represents 5-6 mice) | | | |
| Proteins | Genes | 1 to 6 | 7 to 12 | 13 to 17 | 18 to 22 |
| DnaK (HSP70) | FTT1269 | x | | x | x |
| TM protein (OmpH) | FTT1747 | x | x | x | x |
| HSP60 (Cpn60) | FTT1696 | x | | | x |
| TM protein | FTT0975 | x | | x | x |
| 17 kd Protein (IpnA) | FTT0901 | | | | |
| | FTT0901 | x | | | x |
| | FTT1477 | | | | |
| biotin carboxyl | FTT0472 | x | | | x |
| carrier | | | | | |
| | FTT0264 | | | | |

TABLE 6
Antigenic proteins detected by serum from mice challenged with Schu 84.

| | | Murine Schus4 challenge Mice Pools | | | |
|---|---|---|---|---|---|
| | | (each col. Represents serum | | | |
| | | from 5-6 mice) | | | |
| Proteins | Genes | 1 to 6 | 7 to 12 | 13 to 17 | 18 to 22 |
| DnaK (HSP70) | FTT1269 | x | x | x | x |
| TM protein (OmpH) | FTT1747 | x | x | x | x |
| HSP60 (Cpn60) | FTT1696 | x | x | x | x |
| 1272 SS TM protein | FTT0975 | x | x | | x |
| 17 kd Protein (IpnA) | FTT0901 | x | | | |
| | FTT0901 | x | | | |
| | FTT1477 | x | x | | |
| biotin carboxyl carrier | FTT0472 | | | | x |
| | FTT0264 | | | | x |

The tables show that the mice challenged with a virulent organism produced more antibodies than those challenged only with the non-infectious strain, and that certain antibodies were produced very consistently regardless of which strain was used to immunize the mice.

By way of example only and without limiting the scope of proteins or DNA sequences encompassed by the invention, some of the closest variants and orthologs for some of the immunoactive proteins identified by the present method include:

FTT1269 (DnaK):
Pseudomonas aeruginosa PAO1
Pseudomonas putida KT2440
Legionella pneumophila
Coxiella bumetii strain RSA 493
Legionella pneumophila str. Lens
Legionella pneumophila str. Paris
Coxiella bumetii dnaK
Legionella pneumophila grpE, dnaK, dnaJ
Salmonella enterica
Salmonella enterica serovar Typhi (Salmonella typhi) strain CT18

FTT1696 (Hsp60):
Acinetobacter sp. ADP1
Xenorhabdus nematophila GroEL-like protein gene
Vibrio cholerae O1 biovar eltor str. N16961 chromosome I
Pseudomonas aeruginosa PAO1
Klebsiella pneumoniae gene for GroES protein homologue, GroEL protein homologue
Enterobacter agglomerans gene for GroES protein homologue, GroEL protein homologue
Enterobacter asburiae gene for GroES protein homologue, GroEL protein homologue
Pseudomonas aeruginosa GroEL (mopA) gene
Enterobacter aerogenes gene for GroES protein homologue, GroEL protein homologue
Pseudoalteromonas sp. PS1M3 gene for GroES, GroEL

FTT0901 (17 kd protein):
Francisella endosymbiont of Dennacentor albipictus clone T1G 17 kDa lipoprotein gene
Francisella endosymbiont of Dermacentor variabilis clone 01-109 17 kDa lipoprotein gene
Francisella endosymbiont of Dermacentor occidentalis clone 02-241 17 kDa lipoprotein gene
Francisella endosymbiont of Dermacentor hunteri clone 01-113 17 kDa lipoprotein gene
Francisella endosymbiont of Dermacentor andersoni clone 01-151-1 17 kDa lipoprotein gene
Francisella endosymbiont of Dermacentor andersoni clone 01-171 17 kDa lipoprotein gene
Francisella endosymbiont of Dermacentor nitens clone DnT2-1 17 kDa lipoprotein gene
Francisella endosymbiont of Dermacentor hunteri clone 02-249 17 kDa lipoprotein gene
Francisella endosymbiont of Dermacentor hunteri clone 01-112 17 kDa lipoprotein gene
Francisella endosymbiont of Dermacentor andersoni clone 02-31 17 kDa lipoprotein gene

FTT1477c:
Pseudomonas putida KT2440
Pseudomonas syringae pv. tomato str. DC3000
Pseudomonas aeruginosa PA01
Xanthomonas axonopodis pv. citri str. 306
Xanthomonas campestris pv. campestris str. ATCC 33913
Photobacterium profundum SS9
Methylococcus capsulatus str.
Bath Legionella pneumophila str.
Paris Legionella pneumophila str. Lens
Bradyrhizobium japonicum USDA 110

DNA [0202] FTT0472 (biotin carboxyl carrier):
Pseudomonas aeruginosa PA01
Pseudomonas aeruginosa biotin carboxyl carrier protein and biotin
carboxylase (accB and accC) genes
Legionella pneumophila subsp. pneumophila str. Philadelphia 1
Legionella pneumophila str. Paris
Pasteurella multocida subsp. multocida str. Pm70
Legionella pneumophila str. Lens
Methylococcus capsulatus str.
Bath Shigella flexneri 2a str.
Salmonella typhimurium LT2
Shigella flexneri 2a str. 2457T

### EXAMPLE 14

### Antigenic Proteins from Mycobacterium Tuberculosis

Following the methods described above using the proteins of Example 1C from Mycobacterium tuberculosis H37Rv, the following antigenic proteins were identified (selected known variants and orthologs are also presented as non-limiting examples):

Rv3333c (hypothetical proline rich protein)

| | |
|---|---|
| Variants/orthologs: | Mb2765c (M. bovis) |
| | ML0981 (M. leprae) |

Rv0440 (60 kDa chaperonin)

| | |
|---|---|
| Variants/orthologs: | Mb0448 (M. bovis) |
| | ML0317 (M. leprae) |

Rv1860 (alanine and proline rich secreted protein APA)

| | |
|---|---|
| Variants/orthologs: | Mb1891 (M. bovis) |

Rv3763 (19 kDa liproprotein antigen precursor LPQH)

| | |
|---|---|
| Variants/orthologs: | Mb3789 (M. bovis) |
| | ML1966 (M. leprae) |

Rv3874 (10 kDa culture filtrate antigen ESXB)

| | |
|---|---|
| Variants/orthologs: | Mb2765c (M. bovis) |

Rv3875 (6 kDa early secretory antigenic target ESXA)

| | |
|---|---|
| Variants/orthologs: | Mb3905 (M. bovis) |

### EXAMPLE 15

### Antigenic Proteins from Mycobacterium Tuberculosis

Proteins from 312 expressed genes of Mycobacterium tuberculosis H37Rv were tested with sera from rabbits, mice, and monkeys using the methods described above and proteins from the genes obtained in Example 1C. The following table lists the antigens detected using serum from each species: each protein is identified by the locus tag for the corresponding gene that is used in the publicly available GenBank database. The serum of non-infected animals reacted to all of the antigens listed; the antigens that were only detected by serum from TB-infected animals are listed in boldface and highlighted.

TABLE 7

| Rabbit | Mouse | Monkey |
|---|---|---|
| Rv0040 | Rv0040 | Rv0440 |
| Rv0292 | Rv0102 | Rv0475 |
| Rv0432 | Rv0292 | Rv0577 |
| Rv0674 | Rv0366c | Rv1801 |
| Rv0867c | Rv0432 | Rv1860 |
| Rv1004c | Rv0440 | Rv1980c |
| Rv1157c | Rv0467 | Rv2220 |
| Rv1184c | Rv0526 | Rv2744c |
| Rv1310 | Rv0538 | Rv2873 |
| Rv1435c | Rv0545c | Rv2875 |
| Rv1620c | Rv0685 | Rv3270 |
| Rv1733c | Rv0798c | Rv3333c |
| Rv1801 | Rv0847 | Rv3418c |
| Rv1837c | Rv0886 | Rv3763 |
| Rv1860 | Rv0916c | Rv3873 |
| Rv2031c | Rv0934 | Rv3874 |
| Rv2190c | Rv1004c | Rv3875 |
| Rv2195 | Rv1244 | Rv3875 & Rv3874 |
| | | Fusion |
| Rv2253 | Rv1307 | Rv3881c |
| Rv2376c | Rv1311 | |
| Rv2700 | Rv1435c | |
| Rv2721c | Rv1451 | |
| Rv2744c | Rv1566c | |
| Rv2744c′ | Rv1620c | |
| Rv2864c | Rv1623c_1 | |
| Rv3270 | Rv1686c | |
| Rv3333c | Rv1733c | |
| Rv3449 | Rv1737c | |
| Rv3873 | Rv1860 | |
| | Rv1906c | |
| | Rv1926c | |
| | Rv1984c | |
| | Rv2007c | |
| | Rv2031c | |
| | Rv2193 | |
| | Rv2195 | |
| | Rv2196 | |
| | Rv2253 | |
| | Rv2376c | |
| | Rv2389c | |
| | Rv2446c | |
| | Rv2495c | |
| | Rv2620c | |
| | Rv2700 | |
| | Rv2744c | |
| | Rv2873 | |
| | Rv2875 | |
| | Rv3217c | |
| | Rv3270 | |
| | Rv3330 | |
| | Rv3333c | |
| | Rv3390 | |
| | Rv3418c | |
| | Rv3524 | |
| | Rv3705c | |
| | Rv3714c | |
| | Rv3803c | |
| | Rv3828c | |
| | Rv3841 | |
| | Rv3846 | |
| | Rv3873 | |
| | Rv3874 | |
| | Rv3875 | |
| | Rv3881c | |
| | Rv3914 | |

### EXAMPLE 16

### Tuberculosis Vaccines and Diagnostic Tests

From the data set obtained in Example 15, a cocktail of proteins or nucleic acids encoding proteins is selected for a vaccine composition. A tuberculosis diagnostic test or vaccine cocktail based on these results comprises at least three of the following genes or the corresponding peptides, and may include four or more, or five or more, or most or all of these: Rv0440, Rv0467, Rv0475, Rv0538, Rv0674, Rv0685, Rv0798c, Rv0916c, Rv0934, Rv1801, Rv1860, Rv1926c, Rv1980c, Rv1984c, Rv2007c, Rv2031c, Rv2190c, Rv2220, Rv2376c, Rv2389c, Rv2446c, Rv2744c, Rv2873, Rv2875, Rv2875, Rv3270, Rv3330, Rv3333c, Rv3418c, Rv3763, Rv3803c, Rv3828c, Rv3846, Rv3874, Rv3875, Rv3881c, and Rv3914. Especially suitable antigens include those that were reactive specifically to semm from infected animals of multiple species, which include Rv0440, Rv1801, Rv2031c, Rv2376c, Rv2875, and Rv3875. Also of special interest are those antigens that were specifically recognized by serum from infected monkeys, including Rv0440, Rv0475, Rv1801, Rv1980c, Rv2220, Rv2873, Rv2875, Rv3270, Rv3763, and Rv3875. The vaccine or diagnostic test may therefore comprise two or more, or three or more, or more than three proteins or nucleic acids selected from either of these groups of antigens.

This vaccine is administered using the excipients, compositions and methods disclosed herein to immunize a human subject at risk for tuberculosis, provided the subject's immune system is not compromised.

TABLE 8

| | VACV-COP | | | | |
|---|---|---|---|---|---|
| Locus Name | Ortholog | SIZE | STRAND | START | FINISH |
| VACWR129 | A10L | 891 | − | 121844 | 119169 |
| VACWR130 | A11R | 318 | + | 121859 | 122815 |
| VACWR131 | A12L | 192 | − | 123395 | 122817 |
| VACWR132 | A13L | 70 | − | 123631 | 123419 |
| VACWR133 | A14L | 90 | − | 124011 | 123739 |
| VACWR135 | A15L | 94 | − | 124463 | 124179 |
| VACWR136 | A16L | 377 | − | 125580 | 124447 |
| VACWR137 | A17L | 203 | − | 126194 | 125583 |
| VACWR138 | A18R | 493 | + | 126209 | 127690 |
| VACWR139 | A19L | 77 | − | 127904 | 127671 |
| VACWR119 | A1L | 150 | − | 110357 | 109905 |
| VACWR141 | A20R | 426 | + | 128257 | 129537 |
| VACWR140 | A21L | 117 | − | 128258 | 127905 |
| VACWR142 | A22R | 187 | + | 129467 | 130030 |
| VACWR143 | A23R | 382 | + | 130050 | 131198 |
| VACWR144 | A24R | 1164 | + | 131195 | 134689 |
| VACWR145 | A25L | 65 | − | 134891 | 134694 |
| VACWR146 | A26L-a | 154 | − | 135324 | 134860 |
| VACWR148 | ATI locus proteinr | | | 136239 | 138416 |
| VACWR149 | A26L-b | 500 | − | 139963 | 138461 |
| VACWR150 | A27L | 110 | − | 140345 | 140013 |
| VACWR151 | A28L | 146 | − | 140786 | 140346 |
| VACWR152 | A29L | 305 | − | 141704 | 140787 |
| VACWR120 | A2L | 224 | − | 111052 | 110378 |
| VACWR153 | A30L | 77 | − | 141900 | 141667 |
| VACWR154 | A31R | 124 | + | 142060 | 142434 |
| VACWR155 | A32L | 270 | − | 143213 | 142401 |
| VACWR156 | A33R | 185 | + | 143331 | 143888 |
| VACWR157 | A34R | 168 | + | 143912 | 144418 |
| VACWR158 | A35R | 176 | + | 144462 | 144992 |
| VACWR159 | A36R | 221 | + | 145059 | 145724 |
| VACWR160 | A37R | 263 | + | 145788 | 146579 |
| VACWR162 | A38L | 277 | − | 147687 | 146854 |
| VACWR164 | A39R | 142 | + | 148474 | 148902 |
| VACWR122 | A3L | 644 | − | 113228 | 111294 |
| VACWR165 | A40R | 159 | + | 148928 | 149407 |
| VACWR166 | A41L | 219 | − | 150164 | 149505 |
| VACWR167 | A42R | 133 | + | 150328 | 150729 |
| VACWR168 | A43R | 194 | + | 150767 | 151351 |
| VACWR170 | A44L | 346 | − | 152733 | 151693 |
| VACWR171 | A45R | 125 | + | 152780 | 153157 |
| VACWR172 | A46R | 240 | + | 153147 | 153869 |
| VACWR173 | A47L | 252 | − | 154675 | 153917 |
| VACWR174 | A48R | 227 | + | 154706 | 155389 |
| VACWR175 | A49R | 162 | + | 155437 | 155925 |
| VACWR123 | A4L | 281 | − | 114126 | 113281 |
| VACWR176 | ASOR | 552 | + | 155958 | 157616 |
| VACWR177 | A51R | 334 | + | 157669 | 158673 |
| VACWR178 | A52R | 190 | + | 158743 | 159315 |
| VACWR179 | A53R | 103 | + | 159621 | 159932 |
| VACWR180 | A55R | 564 | + | 160439 | 162133 |
| VACWR181 | A56R | 314 | + | 162183 | 163127 |
| VACWR182 | A57R | 151 | + | 163272 | 163727 |
| VACWR124 | ASR | 164 | + | 114164 | 114658 |
| VACWR125 | A6L | 372 | − | 115773 | 114655 |
| VACWR126 | A7L | 710 | − | 117929 | 115797 |
| VACWR127 | ABR | 288 | + | 117983 | 118849 |
| VACWR128 | A9L | 108 | − | 119168 | 118842 |
| VACWR192 | B10R | 166 | + | 171672 | 172172 |
| VACWR193 | B11R | 72 | + | 172244 | 172462 |
| VACWR194 | B12R | 283 | + | 172529 | 173380 |
| VACWR195 | B14R | 345 | + | 173473 | 174510 |
| VACWR196 | B15R | 149 | + | 174585 | 175034 |
| VACWR197 | B16R | 326 | + | 175118 | 176098 |
| VACWR198 | B17L | 340 | − | 177166 | 176144 |
| VACWR199 | B18R | 574 | + | 177306 | 179030 |
| VACWR203 | B18R | 309 | + | 180898 | 181827 |
| VACWR200 | B19R | 351 | + | 179102 | 180157 |
| VACWR183 | B1R | 300 | + | 163878 | 164780 |
| VACWR202 | B20R | 53 | + | 180482 | 180643 |
| VACWR184 | B2R | 219 | + | 164870 | 165529 |
| VACWR185 | B3R | 167 | + | 165565 | 166068 |
| VACWR186 | B4R | 558 | + | 166594 | 168270 |
| VACWR187 | BSR | 317 | + | 168374 | 169327 |
| VACWR188 | B6R | 173 | + | 169409 | 169930 |
| VACWR189 | B7R | 182 | + | 169968 | 170516 |
| VACWR190 | BBR | 272 | + | 170571 | 171389 |
| VACWR191 | B9R | 77 | + | 171476 | 171709 |
| VACWR209 | C10L | 331 | + | 185807 | 186802 |
| VACWR210 | C11R | 140 | − | 187379 | 186957 |
| VACWR205 | C12L | 353 | + | 182511 | 183572 |
| VACWR206 | C14L | 190 | + | 183734 | 184306 |
| VACWR017 | C17L | 71 | − | 12682 | 12467 |
| VACWR008 | C19L | 112 | − | 7060 | 6722 |
| VACWR027 | C1L | 229 | − | 21832 | 21143 |
| VACWR212 | C20L | 109 | + | 188295 | 188624 |
| VACWR006 | C21L | 64 | − | 6155 | 5961 |
| VACWR004 | C22L | 122 | − | 5460 | 5092 |
| VACWR001 | C23L | 244 | − | 4375 | 3641 |
| VACWR026 | C2L | 512 | − | 21073 | 19535 |
| VACWR025 | C3L | 263 | − | 19468 | 18677 |
| VACWR024 | C4L | 316 | − | 18610 | 17660 |
| VACWR023 | C5L | 204 | − | 17597 | 16983 |
| VACWR022 | C6L | 151 | − | 16856 | 16401 |
| VACWR021 | C7L | 150 | − | 16168 | 15716 |
| VACWR020 | C8L | 177 | − | 15644 | 15111 |
| VACWR019 | C9L | 634 | − | 15068 | 13164 |
| VACWR115 | D10R | 248 | + | 104655 | 105401 |
| VACWR116 | D11L | 631 | − | 107297 | 105402 |
| VACWR117 | D12L | 287 | − | 108195 | 107332 |
| VACWR118 | D13L | 551 | − | 109881 | 108226 |
| VACWR106 | D1R | 844 | + | 93948 | 96482 |
| VACWR107 | D2L | 146 | − | 96881 | 96441 |
| VACWR108 | D3R | 237 | + | 96874 | 97587 |
| VACWR109 | D4R | 218 | + | 97587 | 98243 |
| VACWR110 | D5R | 785 | + | 98275 | 100632 |
| VACWR111 | D6R | 637 | + | 100673 | 102586 |
| VACWR112 | D7R | 161 | + | 102613 | 103098 |
| VACWR113 | D8L | 304 | − | 103975 | 103061 |
| VACWR114 | D9R | 213 | + | 104017 | 104658 |
| VACWR066 | E10R | 95 | + | 56688 | 56975 |
| VACWR067 | E11L | 129 | − | 57359 | 56970 |
| VACWR057 | E1L | 479 | − | 45443 | 44004 |
| VACWR058 | E2L | 737 | − | 47653 | 45440 |
| VACWR059 | E3L | 190 | − | 48352 | 47780 |
| VACWR060 | E4L | 259 | − | 49187 | 48408 |
| VACWR061 | ESR | 341 | + | 49236 | 50261 |
| VACWR062 | E6R | 567 | + | 50398 | 52101 |
| VACWR063 | E7R | 166 | + | 52183 | 52683 |
| VACWR064 | ESR | 273 | + | 52808 | 53629 |
| VACWR065 | E9L | 1006 | − | 56656 | 53636 |
| VACWR049 | F10L | 439 | − | 37778 | 36459 |
| VACWR050 | F11L | 348 | − | 38847 | 37801 |
| VACWR051 | F12L | 635 | − | 40797 | 38890 |
| VACWR052 | F13L | 372 | − | 41949 | 40831 |
| VACWR053 | F14L | 73 | − | 42188 | 41967 |
| VACWR054 | F15L | 147 | − | 42903 | 42460 |
| VACWR055 | F16L | 231 | − | 43639 | 42944 |
| VACWR056 | F17R | 101 | + | 43702 | 44007 |
| VACWR040 | F1L | 226 | − | 31026 | 30346 |
| VACWR041 | F2L | 147 | − | 31481 | 31038 |
| VACWR042 | F3L | 480 | − | 32947 | 31505 |
| VACWR043 | F4L | 319 | − | 33917 | 32958 |
| VACWR044 | FSL | 322 | − | 34917 | 33949 |
| VACWR045 | F6L | 74 | − | 35171 | 34947 |
| VACWR046 | F7L | 80 | − | 35429 | 35187 |
| VACWR047 | FSL | 65 | − | 35774 | 35577 |
| VACWR048 | F9L | 212 | − | 36472 | 35834 |
| VACWR078 | G1L | 591 | − | 70752 | 68977 |
| VACWR080 | G2R | 220 | + | 71078 | 71740 |
| VACWR079 | G3L | 111 | − | 71084 | 70749 |
| VACWR081 | G4L | 124 | − | 72084 | 71710 |
| VACWR082 | G5R | 434 | + | 72087 | 73391 |
| VACWR084 | G6R | 165 | + | 73592 | 74089 |
| VACWR085 | G7L | 371 | − | 75169 | 74054 |
| VACWR086 | GSR | 260 | + | 75200 | 75982 |
| VACWR087 | G9R | 340 | + | 76002 | 77024 |
| VACWR099 | H1L | 171 | − | 87737 | 87222 |
| VACWR100 | H2R | 189 | + | 87751 | 88320 |
| VACWR101 | H3L | 324 | − | 89297 | 88323 |
| VACWR102 | H4L | 795 | − | 91685 | 89298 |
| VACWR103 | HSR | 203 | + | 91871 | 92482 |
| VACWR104 | H6R | 314 | + | 92483 | 93427 |
| VACWR105 | H7R | 146 | + | 93464 | 93904 |
| VACWR070 | I1LL | 312 | − | 60804 | 59866 |
| VACWR071 | I2L | 73 | − | 61032 | 60811 |
| VACWR072 | I3L | 269 | − | 61842 | 61033 |
| VACWR073 | I4L | 771 | − | 64240 | 61925 |
| VACWR074 | I5L | 79 | − | 64506 | 64267 |
| VACWR075 | I6L | 382 | − | 65673 | 64525 |
| VACWR076 | I7L | 423 | − | 66937 | 65666 |
| VACWR077 | I8R | 676 | + | 66943 | 68973 |
| VACWR093 | J1R | 153 | + | 80247 | 80708 |
| VACWR094 | J2R | 177 | + | 80724 | 81257 |
| VACWR095 | J3R | 333 | + | 81323 | 82324 |
| VACWR096 | J4R | 185 | + | 82239 | 82796 |
| VACWR097 | JSL | 133 | − | 83258 | 82857 |
| VACWR098 | J6R | 1286 | + | 83365 | 87225 |
| VACWR032 | K1L | 284 | − | 25925 | 25071 |
| VACWR033 | K2L | 369 | − | 27256 | 26147 |
| VACWR034 | K3L | 88 | − | 27572 | 27306 |
| VACWR035 | K4L | 424 | − | 28898 | 27624 |
| VACWR037 | KSL | 134 | − | 29479 | 29075 |
| VACWR038 | K6L | 81 | − | 29693 | 29448 |
| VACWR039 | K7R | 149 | + | 29832 | 30281 |
| VACWR088 | L1R | 250 | + | 77025 | 77777 |
| VACWR089 | L2R | 87 | + | 77809 | 78072 |
| VACWR090 | L3L | 350 | − | 79114 | 78062 |
| VACWR091 | L4R | 251 | + | 79139 | 79894 |
| VACWR092 | LSR | 128 | + | 79904 | 80290 |
| VACWR030 | M1L | 472 | − | 24296 | 22878 |
| VACWR031 | M2L | 220 | − | 24936 | 24274 |
| VACWR028 | N1L | 117 | − | 22172 | 21819 |
| VACWR029 | N2L | 175 | − | 22836 | 22309 |
| VACWR068 | O1L | 666 | − | 59346 | 57346 |
| VACWR069 | O2L | 108 | − | 59720 | 59394 |

The foregoing examples are intended only to illustrate certain embodiments of the invention and are not to be construed as limitations. Those variations that would be apparent to one of ordinary skill are also included within the scope of the present invention. One of ordinary skill will recognize that many aspects and embodiments of the invention described herein may be combined, and the invention expressly includes such combinations of the various aspects and embodiments described.

1 to 67. (canceled)

68. A method of making an array,
wherein the array comprises a plurality of different, individual and non-pure recombinant proteins and/or peptides of at least one pathogen, infectious agent or prokaryote having a known genome affixed on a plurality of distinct, individually addressable locations on a surface of a substrate, a plate or chip to produce an array of distinct, individually addressable locations,
wherein the plurality of recombinant proteins and/or peptides comprises at least about 100 proteins and/or peptides and represents at least about 50% of the genome of the pathogen or infectious agent, or if the known genome is the genome of an infectious agent or the genome of the prokaryote, the plurality of different, individual and non-pure recombinant proteins and/or peptides comprises at least about 10% of the proteins and peptides expressed by the pathogen, infectious agent or prokaryote having a known genome,
wherein the method of making the array comprises:
(a) providing a plurality of linearized expression vectors;
(b) providing a plurality of amplification primers comprising sequences capable of amplifying a desired number of open reading frame (ORF) coding sequences encoding the plurality of recombinant proteins and/or peptides expressed by the pathogen, infectious agent or prokaryote having a known genome,
wherein each of the plurality of amplification primers contains both a sequence complementary to an end portion of one of the desired number of the ORF coding sequences and an adapter being homologous to a sequence provided on a linearized expression vector or on the plurality of linearized expression vectors,
and using an amplification technique to amplify individually a desired number of open reading frames (ORF) coding sequences to obtain a plurality of individually amplified segments,
(c) providing a recombinase-containing host cell;
(d) co-transfecting into the recombinase-containing host cell the plurality of amplified products and the plurality of linearized expression vectors;
(e) culturing the co-transfected host cell for sufficient time on or in a suitable medium to allow homologous recombination of the plurality of amplified products and the plurality of linearized expression vectors in vivo, wherein the plurality of linearized expression vectors and the plurality of amplified products are ligated by homologous recombination in vivo in the cells to generate a plurality of ligated expression vectors;
(f) extracting or harvesting from the host cell the plurality of ligated expression vectors;
(g) translating the plurality of ligated expression vectors in:
(1) a cellular derived system, which is a cell-free in vitro translation system, to obtain a peptide- and/or protein-containing mixture, or
(2) a suitable host cell in vivo,
wherein the translation generates a peptide and/or protein containing or comprising the peptide- and/or protein-containing mixture, and
(g) spotting or placing the peptide- and/or protein-containing mixture directly onto a solid support to generate the array of different, individual and non-pure recombinant proteins and/or peptides of at least one pathogen, infectious agent or prokaryote having a known genome.

69. The method of claim 68, wherein the plurality of different, individual and non-pure recombinant proteins and/or peptides represents at least about 70% of the genome of the pathogen or infectious agent.

70. The method of claim 68, wherein if the known genome is the genome of an infectious agent or the genome of any prokaryote, the plurality of different, individual and non-pure recombinant proteins and/or peptides represents at least about 20% of the proteins and peptides expressed by the pathogen or prokaryote having a known genome.

71. The method of claim 68, wherein the pathogen, infectious agent or prokaryote is selected from the group consisting of a Vaccinia virus, a human Papillomavirus, a West Nile virus, Francisella tularensis, Burkholderia pseudomallei, Plasmodium falciparum, and Mycobacterium tuberculosis.

72. The method of claim 68, wherein a portion of the cell-free in vitro translation system comprises a supernatant of the cell-free expression extract.

73. The method of claim 68, wherein the expression vector is a plasmid.

74. The method of claim 68, wherein the cell-free in vitro translation system is a prokaryotic or a eukaryotic cell-free in vitro translation system.

75. The method of claim 74, wherein the prokaryotic cell-free in vitro translation system is a bacterial cell-free in vitro translation system

76. The method of claim 74, wherein the eukaryotic cell-free in vitro translation system is a mammalian, a plant, an insect or a human reticulocyte cell-free in vitro translation system.

77. The method of claim 68, wherein the ratio of expression vector to cells in the transfection reaction is adjusted to be at most 100 ng/million cells, or 1 to 10 ng /million cells.

78. The method of claim 68, wherein the solid support is a microtiter plate, a chip or a nitrocellulose substrate.

79. The method of claim 68, wherein the pathogen is selected from the group consisting of a Vaccinia virus, human papilloma virus, West Nile virus, Francisella tularensis, Burkholderia pseudomallei, Plasmodium falciparum, and Mycobacterium tuberculosis.

80. The method of claim 70, wherein if the known genome is the genome of an infectious agent or the genome of any prokaryote, the plurality of different, individual and non-pure recombinant proteins and/or peptides represents at least 50% of the proteins and peptides expressed by the pathogen or prokaryote having a known genome.

81. The array of claim 80, wherein if the known genome is the genome of an infectious agent or the genome of any prokaryote, the plurality of different, individual and non-pure recombinant proteins and/or peptides represents at least 75% of the proteins and peptides expressed by the pathogen or prokaryote having a known genome.

82. The array of claim 81, wherein if the known genome is the genome of an infectious agent or the genome of any prokaryote, the plurality of different, individual and non-pure recombinant proteins and/or peptides represents at least 90% of the proteins and peptides expressed by the pathogen or prokaryote having a known genome.

83. The method of claim 68, wherein the amplification technique comprises a polymerase chain reaction (PCR).

84. The method of claim 68, wherein the translating of the plurality of ligated expression vectors obtained in a cellular derived system is in a cell-free in vitro translation system.
